(19) United States
(12) Patent Application Publication
(10) Pub. No.: US 2024/0051940 A1
(43) Pub. Date: Feb. 15, 2024

(54) SULFONAMIDES WITH EGFR INHIBITION ACTIVITIES AND THEIR USE THEREOF

(71) Applicant: ACCUTAR BIOTECHNOLOGY INC., Cranbury, NJ (US)

(72) Inventors: Yimin QIAN, Plainsboro, NJ (US); Robert LUO, New City, NY (US); Wei HE, Zionsville, IN (US); Jie Fan, New York, NY (US)

(73) Assignee: ACCUTAR BIOTECHNOLOGY INC., Cranbury, NJ (US)

(21) Appl. No.: 18/264,308

(22) PCT Filed: Feb. 7, 2022
(86) PCT No.: PCT/US2022/015425

§ 371 Date: Aug. 4, 2023

Related U.S. Application Data
(60) Provisional application No. 63/146,985, filed on Feb. 8, 2021.

(51) Int. Cl. C07D 401/14; C07D 401/12; C07D 471/10; C07D 471/04; C07D 403/14; C07D 491/107; C07D 471/08; C07D 487/08; C07D 487/04; C07D 413/14; C07D 498/10; A61K 45/06

(52) U.S. Cl. CPC C07D 401/14; C07D 401/12; C07D 471/10; C07D 471/04; C07D 403/14; C07D 491/107; C07D 471/08; C07D 487/08; C07D 487/04; C07D 413/14; C07D 498/10; A61K 45/06; C07B 2200/05

(57) ABSTRACT

The disclosure provides sulfonamides based compounds and pharmaceutical compositions thereof, which are useful for inhibiting one or more mutations of EGFR, as well as methods for using such compounds to treat cancer associated with EGFR mutations.

This application claims priority from U.S. Provisional Patent Application No. 63/146,985, filed Feb. 8, 2021, which is hereby incorporated by reference in its entirety.

### FIELD OF THE DISCLOSURE

The present invention relates to novel sulfonamide compounds, or pharmaceutically acceptable salts thereof, which possess anti-tumor activity and are accordingly useful in methods of treatment of the human or animal body. In particular, the present invention relates to compounds which inhibit one or more mutations of the Epidermal Growth Factor Receptor (EGFR), pharmaceutical compositions comprising the compounds, and methods of use therefor.

### BACKGROUND OF THE DISCLOSURE

Protein kinases are a group of enzymes that regulate diverse, important biological processes including, for example, cell growth, proliferation, survival, invasion and differentiation, organ formation, tissue repair and regeneration. Protein kinases exert their physiological functions through catalyzing the phosphorylation of protein and thereby modulating cellular activities. Because protein kinases have profound effects on cells, their activities are highly regulated. Kinases are turned on or off by phosphorylation (sometimes by autophosphorylation), by binding of activator proteins or inhibitor proteins, or small molecules, or by controlling their location in the cell relative to their substrates.

The epidermal growth factor receptor (EGFR; ErbB-1; HER1 in humans) is a member of the ErbB family of receptors, a subfamily of four closely related receptor tyrosine kinases: EGFR (ErbB-1), HER2/c-neu (ErbB-2), Her 3 (ErbB-3) and Her 4 (ErbB-4). EGFR is the cell-surface receptor for members of the epidermal growth factor family (EGF-family) of extracellular protein ligands. Mutations affecting EGFR expression or activity could result in cancer. EGFR is reported deregulated in most solid tumor types, i.e., lung cancer, breast cancer and brain tumor. It is estimated that mutations, amplifications or misregulations of EGFR or family members are implicated in about 30% of all epithelial cancers.

It was reported in 2004 that an activating mutation of EGFR is correlated with a response to gefitinib therapy in non-small cell lung cancer (NSCLC) (Science [2004] Vol. 304, 1497-500 and New England Journal of Medicine [2004] Vol. 350, 2129-39).

It is also known that the above EGFR mutation is largely classified into a sensitizing mutation and a resistant mutation, and a deletion of exon 19 and a L858R point mutation of exon 21 are the most important sensitizing mutations and make up about 85 to 90 percent of a sensitizing mutation, and an exon 19 del mutation is more sensitizing to the tyrosine kinase inhibitors (TKIs). On the other hand, it is known that a T790M point mutation of exon 20 is the most important resistant mutation and is found in at least 50 percent of acquired resistant patients (Clin Cancer Res 2006; 12:6494-6501).

Somatic mutations identified here include an in-frame deletion in exon 19 or an insertion in exon 20, as well as a point mutation in which a single nucleic acid residue is modified within an expressed protein (for example, L858R, G719S, G719C, G719A, L861Q) (Fukuoka et al. JCO 2003; Kris et al JAMA 2003; and Shepherd et al NEJM 2004).

Therapeutic approaches have been developed based on the inhibition of EGFR by either antibody drug or small molecular inhibitor drug, such as gefitinib and erlotinib.

For example, an early clinical effect of gefitinib/erlotinib is observed in NSCLC patients with a EGFR mutation, but a progressive cancer develops in most patients in the end while these patients are receiving a therapy of these drugs. In an early study of recurred samples, a secondary EGFR mutation, T790M, was identified, which made gefitinib and erlotinib to be ineffective inhibitors of EGFR kinase activity (Kobayashi et al NEJM 2005 and Pao et al PLOS Medicine 2005). It was also shown in a follow-up study that the EGFR T790M mutation was found in approximately 50 percent ( 24/48) of tumors derived from patients who acquired a resistance against gefitinib or erlotinib (Kosaka et al CCR 2006; Balak et al CCR 2006; and Engelman et al Science 2007). The secondary genetic modification is caused in a position similar to a ‘gatekeeper’ residue and a secondary resistance allele associated with the same in patients to be treated with a kinase inhibitor (for example, T3151 within ABL in imatinib resistant CML).

Moreover, it is known that EGFR del 19 or EGFR L858R, which are EGFR mutations, is a major cause of non-small cell lung cancer and head and neck cancer, and gefitinib and erlotinib, which are therapeutic drugs of the cancers, were developed. However, when such drugs were administered for cancer patients, an acquired resistance caused by an EGFR secondary mutation based on the structure of the drug was observed. Importantly, it was found that this was a major cause of drug resistance. If first generation inhibitors of EGFR have been used for about ten months on average, an acquired resistance, which is a T790M mutation positioned in a gatekeeper of EGFR kinase, occurs to prevent first generation inhibitors of EGFR exerting a medicinal effect. That is, EGFR dell 19 T790M or EGFR L858R T790M double mutation occurs to prevent conventional therapeutic agents exerting a medicinal effect.

Various third generation new drug candidates showing an effect on an EGFR T790M double mutation were developed. Osimertinib is one example. However, it was reported that resistance caused by triple mutations including C797S occurred (Thress et al, Nature Medicine 2015).

Thus, there is a need for the development of inhibitors showing high inhibition on mutant forms of EGFR (e.g. triple-mutant EGFR) while showing a relatively low inhibition of WT EGFR.

The inventors have found novel sulfonamide compounds that have high potency against, for example, several mutant forms of EGFR while at the same showing relatively low inhibition of WT EGFR.

### SUMMARY OF THE DISCLOSURE

In some embodiments of the invention, provided herein are compound of Formula (I), or a pharmaceutically acceptable salt, or hydrate, or deuterated derivative thereof:

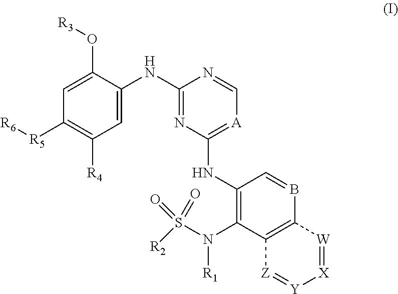

wherein:
W is CH or N;
X is CH or N;
Y is CH or N;
Z is CH or N;
A is N or C—R^{a}, wherein R^{a} is chosen from the group of H, CF₃, halogen, C₁-C₄alkyl, C₃-C₅cycloalkyl, —COOH, and —C(O)OC₁-C₄alkyl, with the proviso that when A is N, the ring containing W, X, Y, and Z may be null;
B is CH or N;
R₁ is H or C₁-C₄alkyl;
R₂ is chosen from —NH₂, C₁-C₄alkyl, and C₃-C₆cycloalkyl;
R₃ is C₁-C₄alkyl, wherein one or more hydrogen atoms are optionally and independently replaced by deuterium or F;
R₄ is chosen from H, C₁-C₄alkyl, C₃-C₅cycloalkyl, vinyl, C₂-alkynyl, and 5- to 12-membered heteroaryl comprising one to three hetero atoms selected from O, N and S, wherein the 5- to 12-membered heteroaryl may be further substituted with one or two C₁-C₃alkyl groups or deuterated C₁-C₃alkyl groups;
R₅ is monocyclic or bicyclic including bridged bicyclic C₃-C₁₂heterocycloalkyl comprising one to three hetero atoms selected from O, N and S, wherein the C₃-C₁₂heterocycloalkyl may be further substituted with one or two groups chosen from halogen, hydroxy, alkoxy, cyano, C₁-C₄alkyl, C₁-C₄haloalkyl, and C₁-C₄dialkylamino;
R₅ is absent or is C₃-C₁₀ heterocycloalkyl including bridged heterobicycles comprising one or two hetero atoms selected from O, N and S, wherein the C₃-C₁₀ heterocycloalkyl may be further substituted with one or two groups chosen from C₁-C₄alkyl, deuterated C₁-C₄alkyl, C₁-C₄alkoxy and C₁-C₄dialkylamino, and wherein the C₁-C₄alkyl may be further substituted with one to three groups chosen from halogen, —CN, hydroxyl, amino group, amide group, sulfoxide, sulfonamide, C₁-C₃alkoxy, and C₁-C₃acyl. In some embodiments, R₆ is a C₃-C₈ heterocycloalkyl, wherein the C₃-C₈ heterocycloalkyl may be further substituted with one or two groups chosen from C₁-C₄alkyl, deuterated C₁-C₄alkyl, C₁-C₄alkoxy and C₁-C₄dialkylamino, and wherein the C₁-C₄alkyl may be further substituted with one to three groups chosen from halogen, —CN, hydroxyl, amino group, amide group, sulfoxide, sulfonamide, C₁-C₃alkoxy, and C₁-C₃acyl.

In some embodiments, R^{a} is chosen from H, CF₃, halogen, C₃-C₅cycloalkyl, and —C(O)OC₁-C₄alkyl.

In some embodiments, R₁ is H or methyl.

In some embodiments, R₂ is chosen from —NH₂, methyl, ethyl, and cyclopropyl.

In some embodiments, R₃ is chosen from methyl, —CD₃, CHF₂, and —CH₂CF₃.

In some embodiments, R₄ is chosen from H, methyl, ethyl, cyclopropyl, vinyl, and

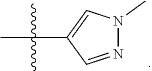 [SMILES: *c1cnn(C)c1]

In some embodiments, R₅ is chosen from

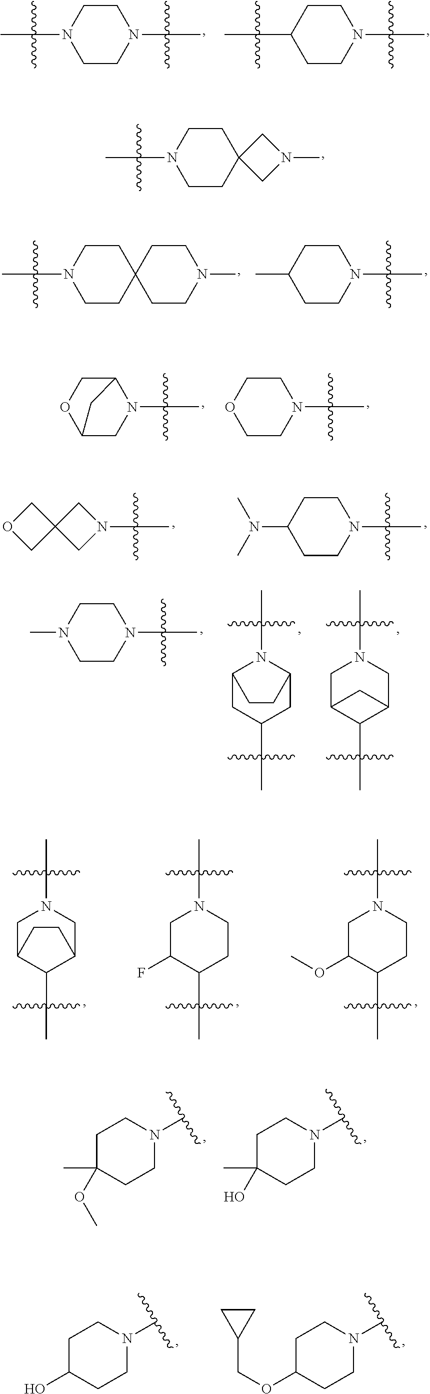

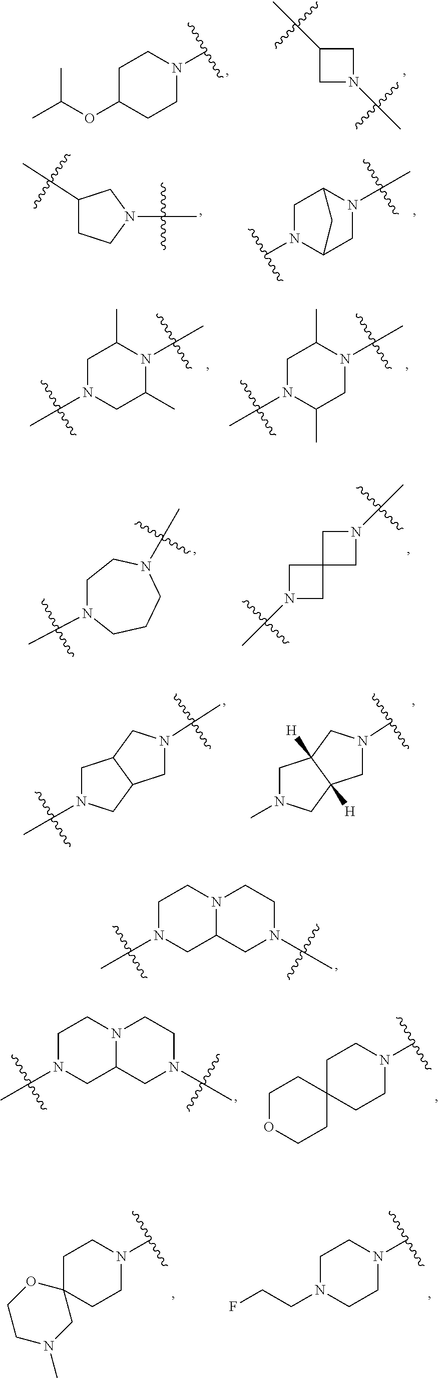

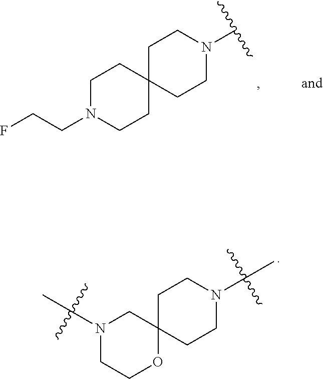 [SMILES: *N1CCC2(CC1)CCN(CCF)CC2 ; *N1CCC2(CC1)CN(*)CCO2]

In some embodiments, R₆ is absent or chosen from

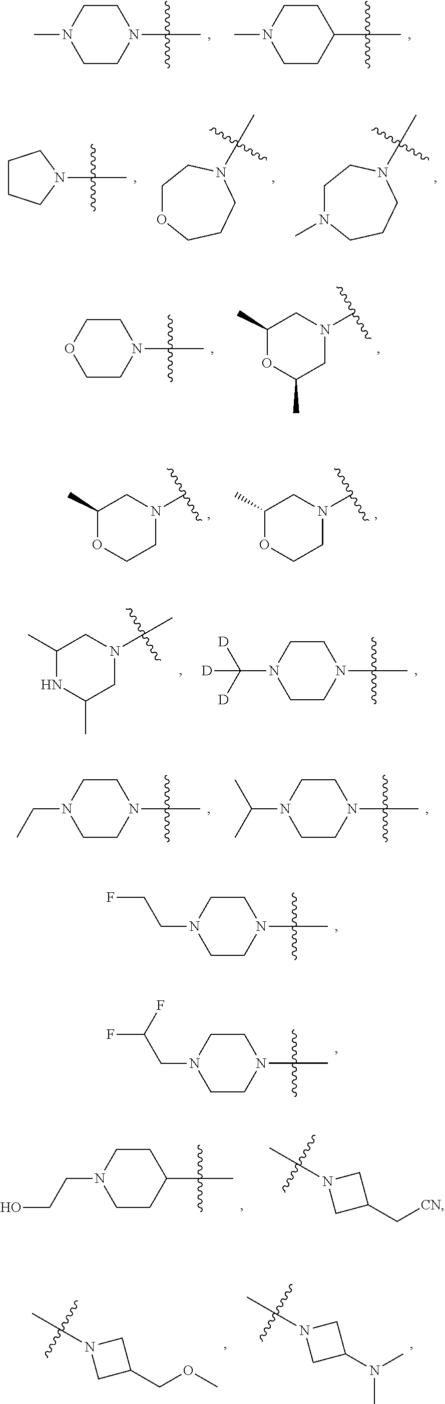

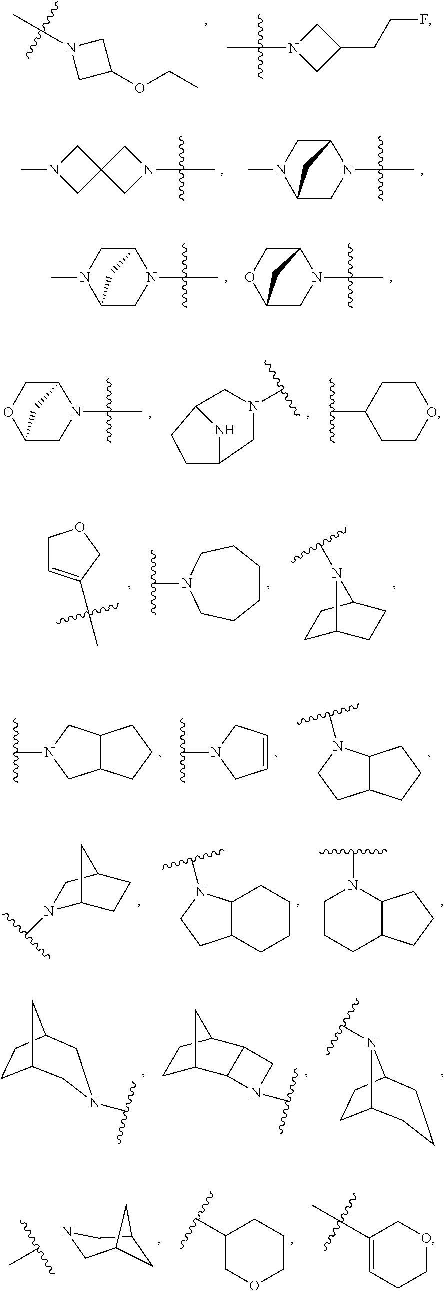

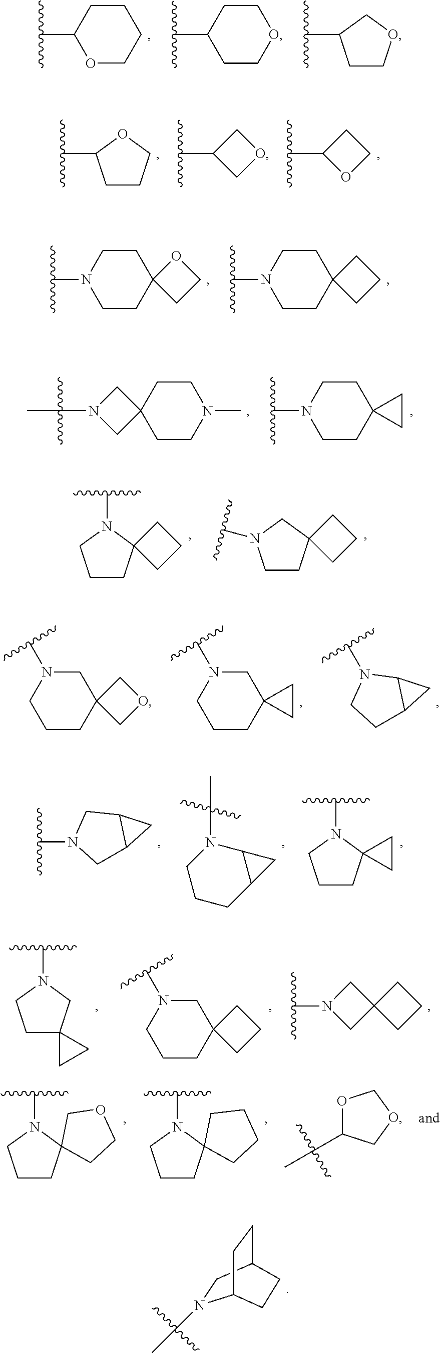

In some embodiments, the compound of Formula (I) may encompass both stereoisomers and a mixture of stereoisomers. In some embodiments, the compound of Formula (I) may encompass both racemic isomers and enantiomeric isomers.

In some embodiments, provided herein is a pharmaceutical composition comprising a compound of Formula (I), or a tautomer, stereoisomer or a mixture of stereoisomers, or a pharmaceutically acceptable salt, or hydrate, or deuterated derivative thereof, and one or more of a pharmaceutically acceptable carrier, a pharmaceutically acceptable vehicle, a pharmaceutically acceptable excipient, or combinations thereof.

Also disclosed herein is a combination therapy comprising a compound of Formula (I), or a tautomer, stereoisomer or a mixture of stereoisomers, or a pharmaceutically acceptable salt, or hydrate, or deuterated derivative thereof, and an additional agent.

In some embodiments, provided herein is the use of a compound of Formula (I), or a tautomer, stereoisomer or a mixture of stereoisomers, or a pharmaceutically acceptable salt, or hydrate, or deuterated derivative thereof, for inhibiting EGFR.

In some embodiments, provided herein is the use of a compound of Formula (I), or a tautomer, stereoisomer or a mixture of stereoisomers, or a pharmaceutically acceptable salt, or hydrate, or deuterated derivative thereof, in the manufacture of a medicament for treating a condition associated with one or more EGFR mutations.

Also disclosed herein is a method for treating a cancer associated with one or more EGFR mutations in a subject in need thereof, comprising administering to the subject an effective amount of a compound of Formula (I), or a tautomer, stereoisomer or a mixture of stereoisomers, or a pharmaceutically acceptable salt, or hydrate, or deuterated derivative thereof, or a pharmaceutical composition comprising a compound of Formula (I), or a tautomer, stereoisomer or a mixture of stereoisomers, or a pharmaceutically acceptable salt, or hydrate, or deuterated derivative thereof.

In at least one embodiment, a pharmaceutically effective amount of a pharmaceutical composition of the present disclosure may be administered to a subject suffering from a cancer associated with one or more EGFR mutations. In some embodiments, the cancer associated with one or more EGFR mutations is chosen from breast cancer, lung cancer, pancreatic cancer, colon cancer, head and neck cancer, renal cell carcinoma, squamous, cell carcinoma, thyroid cancer, gall bladder cancer, thyroid cancer, bile duct cancer, ovarian cancer, endometrial cancer, prostate cancer, and esophageal cancer.

### DETAILED DESCRIPTION

### Definitions

A dash (“-”) that is not between two letters or symbols is used to indicate a point of attachment for a substituent. For example, —CN is attached through the carbon atom.

When a range of values is listed, it is intended to encompass each value and sub-range within the range. For example, “C₁-C₆ alkyl” or “C₁₋₆alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C_{4- 6}, C₄₋₅, and C₅₋₆ alkyl.

The term “acyl” as used herein refers to R—C(O)— groups such as, but not limited to, (alkyl)-C(O)—, (alkenyl)-C(O)—, (alkynyl)-C(O)—, (aryl)-C(O)—, (cycloalkyl)-C(O)—, (heteroaryl)-C(O)—, and (heterocyclyl)-C(O)—, wherein the group is attached to the parent molecular structure through the carbonyl functionality. In some embodiments, it is a C₁₋₁₀ acyl radical which refers to the total number of chain or ring atoms of the, for example, alkyl, alkenyl, alkynyl, aryl, cycloalkyl, or heteroaryl, portion plus the carbonyl carbon of acyl. For example, a C₄-acyl has three other ring or chain atoms plus carbonyl.

The term “alkenyl” as used herein refers to an unsaturated straight or branched hydrocarbon having at least one carbon-carbon double bond, such as a straight or branched group of 2 to 8 carbon atoms, referred to herein as C₂₋₈alkenyl. Exemplary alkenyl groups include, but are not limited to, vinyl, allyl, butenyl, pentenyl, hexenyl, butadienyl, pentadienyl, hexadienyl, 2-ethylhexenyl, 2-propyl, 2-butenyl, and 4-(2-methyl-3-butene)-pentenyl.

The term “alkyl” as used herein refers to a saturated straight or branched hydrocarbon, such as a straight or branched group of 1 to 8 carbon atoms, referred to herein as C1-8alkyl. Exemplary alkyl groups include, but are not limited to, methyl, ethyl, propyl, isopropyl, 2-methyl-1-propyl, 2-methyl-2-propyl, 2-methyl-1-butyl, 3 methyl-1-butyl, 2-methyl-3-butyl, 2,2-dimethyl-1-propyl, 2-methyl-1-pentyl, 3 methyl-1-pentyl, 4-methyl-1-pentyl, 2-methyl-2-pentyl, 3-methyl-2-pentyl, 4 methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl. In some embodiments, “alkyl” is a straight-chain hydrocarbon. In some embodiments, “alkyl” is a branched hydrocarbon.

The term “alkoxy” means a straight or branched chain saturated hydrocarbon containing 1-12 carbon atoms containing a terminal “O” in the chain, e.g., —O(alkyl). Examples of alkoxy groups include, without limitation, methoxy, ethoxy, propoxy, butoxy, t-butoxy, or pentoxy groups.

The term “alkylene” as used herein refers to a divalent alkyl radical. Representative examples of C₁₋₁₀ alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene and n-decylene.

The term “alkynyl” as used herein refers to an unsaturated straight or branched hydrocarbon having at least one carbon-carbon triple bond, such as a straight or branched group of 2 to 8 carbon atoms, referred to herein as C₂₋₈alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “aryl” as used herein refers to a mono-, bi-, or other multi carbocyclic, aromatic ring system with 5 to 14 ring atoms. The aryl group can optionally be fused to one or more rings selected from aryls, cycloalkyls, heteroaryls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include but are not limited to a monocyclic aromatic ring system, wherein the ring comprises 6 carbon atoms, referred to herein as “C₆-aryl.”

The term “cyano” as used herein refers to CN.

The term “cycloalkyl” as used herein refers to a saturated or unsaturated cyclic, bicyclic, or bridged bicyclic hydrocarbon group of 3-16 carbons, or 3-8 carbons, referred to herein as “C₃₋₈cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl (saturated or partially unsaturated), aryl, or heterocyclyl groups, to form a bicycle, tetracycle, etc. The term “cycloalkyl” also includes bridged and spiro-fused cyclic structures which may or may not contain heteroatoms.

The terms “halo” or “halogen” as used herein refer to —F, —Cl, —Br, and/or —I.

“Haloalkyl” means an alkyl group substituted with one or more halogens. Examples of haloalkyl groups include, but are not limited to, trifluoromethyl, difluoromethyl, pentafluoroethyl, trichloromethyl, etc.

The term “heteroatoms,” as used herein, refers to nitrogen (N), oxygen (O), sulfur (S) or phosphorus (P) atoms, wherein the N, S and P can optionally be oxidized to various oxidation states.

The term “heteroaryl” as used herein refers to a mono-, bi-, or multi-cyclic, aromatic ring system containing one or more heteroatoms, for example 1 to 3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Exemplary heteroaryl groups include, but are not limited to, a monocyclic aromatic ring, wherein the ring comprises 2 to 5 carbon atoms and 1 to 3 heteroatoms, referred to herein as “C₂₋₅heteroaryl.” Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups also include, but are not limited to, a bicyclic aromatic ring, wherein the ring comprises 5 to 14 carbon atoms and 1 to 3 heteroatoms, referred to herein as “C₅₋₁₄heteroaryl.” Representative examples of heteroaryl include, but not limited to, indazolyl, indolyl, azaindolyl, indolinyl, benzotriazolyl, benzoxadiazolyl, imidazolyl, cinnolinyl, imidazopyridyl, pyrazolopyridyl, pyrrolopyridyl, quinolinyl, isoquinolinyl, quinazolinyl, quinazolinonyl, indolinonyl, isoindolinonyl, tetrahydronaphthyl, tetrahydroquinolinyl, tetrahydroisoquinolinyl, and the like.

The terms “heterocycle,” “heterocyclyl,” or “heterocyclic” as used herein each refer to a saturated or unsaturated 3- to 18-membered ring containing one, two, three, or four heteroatoms independently selected from nitrogen, oxygen, phosphorus, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

The terms “composition” or “pharmaceutical composition,” as used herein, refers to a mixture of at least one compound, such as a compound Formula (I), or a pharmaceutically acceptable salt thereof, with at least one and optionally more than one other pharmaceutically acceptable chemical components, such as carriers, stabilizers, diluents, dispersing agents, suspending agents, thickening agents, and/or excipients.

The terms “effective amount” or “therapeutically effective amount,” as used herein, refer to a sufficient amount of a compound described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The term “pharmaceutically acceptable,” as used herein, refers to a material, such as a carrier or diluent, which does not abrogate the biological activity or properties of the compounds described herein. Such materials are administered to an individual without causing undesirable biological effects or interacting in a deleterious manner with any of the components of the composition in which it is contained.

The term “carrier,” as used herein, refers to chemical compounds or agents that facilitate the incorporation of a compound described herein into cells or tissues. The term “pharmaceutically acceptable carrier”, as used herein, includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329; Remington: The Science and Practice of Pharmacy, 21^{st} Ed. Pharmaceutical Press 2011; and subsequent versions thereof). Except insofar as any conventional carrier is incompatible with the active ingredient, its use in the therapeutic or pharmaceutical compositions is contemplated.

The term “pharmaceutically acceptable carrier” or “pharmaceutically acceptable vehicle” is used interchangeably and as is known to those skilled in the art, can be any and all solvents, dispersion media, Coatings, surfactants, antioxidants, preservatives (e.g., antimicrobial agents, antifungal agents), isotonic agents, absorption delaying agents, salts, preservatives, drug stabilizers, binders, excipients, disintegrants, lubricant, including sweeteners, flavors, dyes, and the like, and combinations thereof (e.g., Remington's Pharmaceutical Sciences, 18th Ed. Mack Printing Company, 1990, pp. 1289-1329). Except insofar as any conventional carrier is incompatible with the active ingredient, its use in therapeutic or pharmaceutical compositions is contemplated.

The term “pharmaceutically acceptable prodrugs” as used herein represents those prodrugs of the compounds of the present disclosure that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the present disclosure. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

As used herein, the term “inhibit,” “inhibition,” or “inhibiting” refers to the reduction or suppression of a given condition, symptom, or disorder, or disease, or a significant decrease in the baseline activity of a biological activity or process.

As used herein, the term “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically (e.g., through stabilization of a discernible symptom), physiologically, (e.g., through stabilization of a physical parameter), or both. In yet another embodiment, “treat,” “treating,” or “treatment” refers to preventing or delaying the onset or development or progression of the disease or disorder.

As used herein, “cancer” refers to diseases, disorders, and conditions that involve abnormal cell growth with the potential to invade or spread to other parts of the body. Exemplary cancers include, but are not limited to, breast cancer, lung cancer, pancreatic cancer, colon cancer, head and neck cancer, renal cell carcinoma, squamous cell carcinoma, thyroid cancer, gall bladder cancer, thyroid cancer, bile duct cancer, ovarian cancer, endometrial cancer, prostate cancer, or esophageal cancer.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, a subject is “in need of” a treatment if such subject would benefit biologically, medically or in quality of life from such treatment.

The term “administration” or “administering” of the subject compound means providing a compound of the invention, a pharmaceutically acceptable salt, a pharmaceutically acceptable solvate, or solvate thereof to a subject in need of treatment.

The term “combination” as used herein means a product that results from the mixing or combining of more than one active ingredient and includes both fixed and non-fixed combinations of the active ingredients. The term “fixed combination” means that the active ingredients, by way of example, a compound of Formula (I), or a pharmaceutically acceptable salt thereof, and an additional anti-cancer agent, are both administered to a patient simultaneously in the form of a single entity or dosage. The term “non-fixed combination” means that the active ingredients, by way of example, a compound of Formula (I) or a pharmaceutically acceptable salt thereof, and an additional anti-cancer agent, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.). Chemical names were generated using PerkinElmer ChemDraw® Professional, version 17.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, C, ¹³C, ¹⁴C, ¹⁵N, ¹⁸F, ³¹P, ³²P, ³⁵S, ³⁶Cl and ¹²⁵I respectively. The invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the present invention. Isotopically-labeled compounds of the invention can generally be prepared by conventional techniques known to those skilled in the art or by processes analogous to those described in the accompanying Examples and Processes using an appropriate isotopically-labeled reagents in place of the non-labeled reagent previously employed.

### Compounds

In some embodiments, provided herein are compounds of Formula (I), or a tautomer, stereoisomer or a mixture of stereoisomers, or a pharmaceutically acceptable salt, or hydrate, or deuterated derivative thereof:

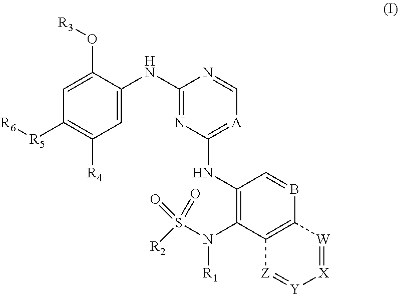

wherein:
W is CH or N;
X is CH or N;
Y is CH or N;
Z is CH or N;
A is N or C—R^{a}, wherein R^{a} is chosen from the group of H, CF₃, halogen, C₁-C₄alkyl, C₃-C₈cycloalkyl, —COOH, and —C(O)OC₁-C₄alkyl, with the proviso that when A is N, the ring containing W, X, Y, and Z may be null;
B is CH or N;
R₁ is H or C₁-C₄alkyl;
R₂ is chosen from —NH₂, C₁-C₄alkyl, and C₃-C₃cycloalkyl;
R₃ is C₁-C₄alkyl, wherein one or more hydrogen atoms are optionally and independently replaced by deuterium or F;
R₄ is chosen from H, C₁-C₄alkyl, C₃-C₅cycloalkyl, vinyl, C₂-alkynyl, and 5- to 12-membered heteroaryl comprising one to three hetero atoms selected from O, N and S, wherein the 5- to 12-membered heteroaryl may be further substituted with one or two C₁-C₃alkyl groups or deuterated C₁-C₃alkyl groups;
R₅ is monocyclic or bicyclic including bridged bicyclic C₃-C₁₂heterocycloalkyl comprising one to three hetero atoms selected from O, N and S, wherein the C₃-C₁₂heterocycloalkyl may be further substituted with one or two groups chosen from halogen, hydroxy, alkoxy, cyano, C₁-C₄alkyl, C₁-C₄haloalkyl, and C₁-C₄dialkylamino;
R₆ is absent or is C₃-C₁₀ heterocycloalkyl including bridged heterobicycles comprising one or two hetero atoms selected from O, N and S, wherein the C₃-C₁₀ heterocycloalkyl may be further substituted with one or two groups chosen from C₁-C₄alkyl, deuterated C₁-C₄alkyl, C₁-C₄alkoxy and C₁-C₄dialkylamino, and wherein the C₁-C₄alkyl may be further substituted with one to three groups chosen from halogen, —CN, hydroxyl, amino group, amide group, sulfoxide, sulfonamide, C₁-C₃alkoxy, and C₁-C₃acyl. In some embodiments, R₆ is a C₃-C₈ heterocycloalkyl, wherein the C₃-C₈ heterocycloalkyl may be further substituted with one or two groups chosen from C₁-C₄alkyl, deuterated C₁-C₄alkyl, C₁-C₄alkoxy and C₁-C₄dialkylamino, and wherein the C₁-C₄alkyl may be further substituted with one to three groups chosen from halogen, —CN, hydroxyl, amino group, amide group, sulfoxide, sulfonamide, C₁-C₃alkoxy, and C₁-C₃acyl. In some embodiments, the C₃-C₁₀ heterocycloalkyl is a bridged heterobicycle, wherein the bridged heterobicycle may be further substituted with one or two groups chosen from C₁-C₄alkyl, deuterated C₁-C₄alkyl, C₁-C₄alkoxy and C₁-C₄dialkylamino, and wherein the C₁-C₄alkyl may be further substituted with one to three groups chosen from halogen, —CN, hydroxyl, amino group, amide group, sulfoxide, sulfonamide, C₁-C₃alkoxy, and C₁-C₃acyl.

In some embodiments, the compound of Formula (I) is not N-(6-((5-bromo-2-((2-methoxy-5-methyl-4-(4-(4-methylpiperazin-1-yl)piperidin-1-yl)phenyl)amino)pyrimidin-4-yl)amino)quinoxalin-5-yl)methanesulfonamide; N-(6-((5-bromo-2-((5-ethyl-2-methoxy-4-(4-(4-methylpiperazin-1-yl)piperidin-1-yl)phenyl)amino)pyrimidin-4-yl)amino)quinoxalin-5-yl)methanesulfonamide; N-(6-((5-bromo-2-((2-methoxy-4-(4-(4-methylpiperazin-1-yl)piperidin-1-yl)phenyl)amino)pyrimidin-4-yl)amino)quinoxalin-5-yl)methanesulfonamide; N-(6-((5-bromo-2-((2-methoxy-5-methyl-4-(4-(4-methylpiperazin-1-yl)piperidin-1-yl)phenyl)amino)pyrimidin-4-yl)amino)quinoxalin-5-yl)-N-methylmethanesulfonamide; N-(6-((5-bromo-2-((5-ethyl-2-methoxy-6-(4-(4-methylpiperazin-1-yl)piperidin-1-yl)pyridin-3-yl)amino)pyrimidin-4-yl)amino)quinoxalin-5-yl)methanesulfonamide; and N-(6-((5-bromo-2-((2-methoxy-5-(1-methyl-1H-pyrazol-4-yl)-4-morpholinophenyl)amino)pyrimidin-4-yl)amino)quinoxalin-5-yl)methanesulfonamide.

In some embodiments, R^{a} is chosen from H, CF₃, halogen, C₃-C₅cycloalkyl, and —C(O)OC₁-C₄alkyl. In some embodiments, R^{a} is CF₃. In some embodiments, R^{a} is Br. In some embodiments, R^{a} is Cl. In some embodiments, R^{a} is cyclopropyl. In some embodiments, R^{a} is —C(O)O-i-Pr.

In some embodiments, R₁ is H or methyl. In some embodiments, R₁ is H. In some embodiments, R₁ is methyl.

In some embodiments, R₂ is chosen from —NH₂, methyl, ethyl, and cyclopropyl. In some embodiments, R₂ is —NH₂. In some embodiments, R₂ is methyl. In some embodiments, R₂ is ethyl. In some embodiments, R₂ is cyclopropyl.

In some embodiments, R₃ is chosen from methyl, —CD₃, CHF₂, and —CH₂CF₃. In some embodiments, R₃ is methyl. In some embodiments, R₃ is —CD₃. In some embodiments, R₃ is —CH₂CF₃.

In some embodiments, R₄ is chosen from H, methyl, ethyl, cyclopropyl, vinyl, and

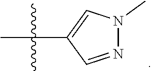 [SMILES: *c1cnn(C)c1]

In some embodiments, R₄ is H. In some embodiments, R₄ is methyl. In some embodiments, R₄ is ethyl. In some embodiments, R₄ is cyclopropyl. In some embodiments, R₄ is vinyl. In some embodiments, R₄ is

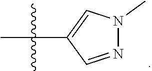 [SMILES: *c1cnn(C)c1]

In some embodiments, R₅ is chosen from

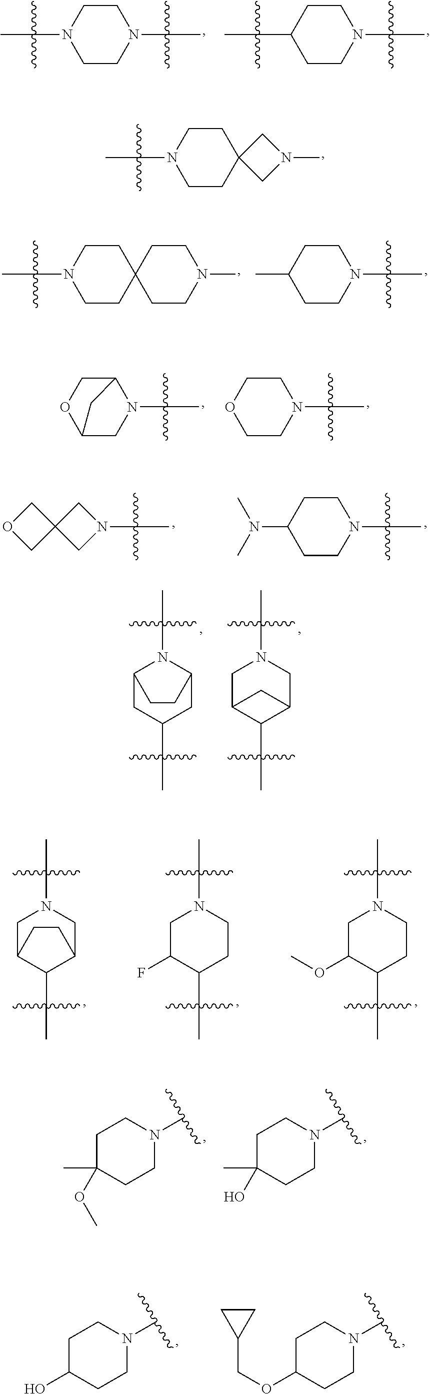

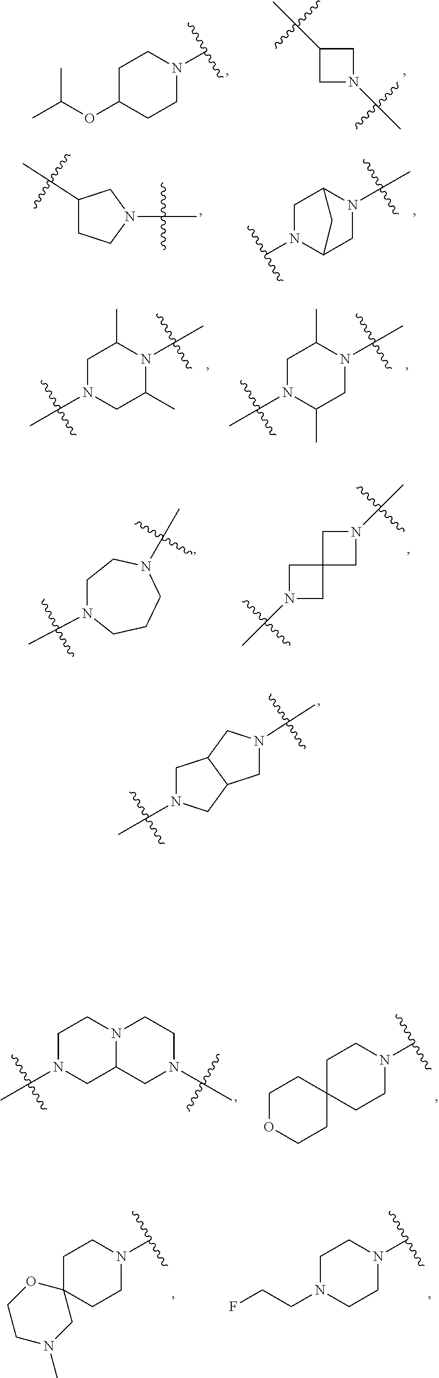

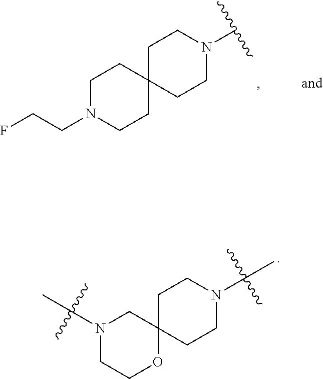 [SMILES: *N1CCC2(CC1)CCN(CCF)CC2 ; *N1CCC2(CC1)CN(*)CCO2]

In some embodiments, R₅ is

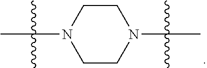 [SMILES: *N1CCN(*)CC1]

In some embodiments, R₅ is

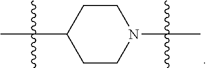 [SMILES: *C1CCN(*)CC1]

In some embodiments, R₅ is

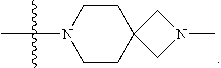 [SMILES: *N1CCC2(CC1)CN(C)C2]

In some embodiments, R₅ is

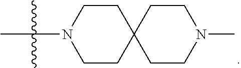 [SMILES: *N1CCC2(CC1)CCN(C)CC2]

In some embodiments, R₅ is

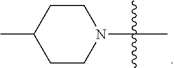 [SMILES: *N1CCC(C)CC1]

In some embodiments, R₅ is

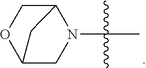 [SMILES: *N1CC2CC1CO2]

In some embodiments, R₅ is

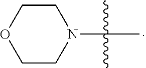 [SMILES: *N1CCOCC1]

In some embodiments, R₅ is

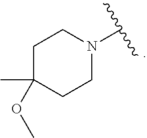 [SMILES: *N1CCC(C)(OC)CC1]

In some embodiments, R₅ is

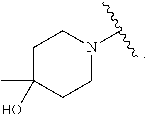 [SMILES: *N1CCC(C)(O)CC1]

In some embodiments, R₅ is

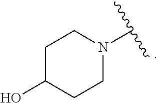 [SMILES: *N1CCC(O)CC1]

In some embodiments, R₅ is

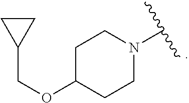 [SMILES: *N1CCC(OCC2CC2)CC1]

In some embodiments, R₅ is

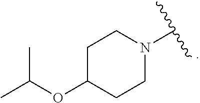 [SMILES: *N1CCC(OC(C)C)CC1]

In some embodiments, R₅ is

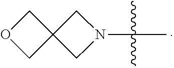 [SMILES: *N1CC2(COC2)C1]

In some embodiments, R₅ is

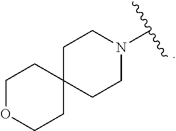 [SMILES: *N1CCC2(CCOCC2)CC1]

In some embodiments, R₅ is

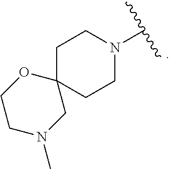 [SMILES: *N1CCC2(CC1)CN(C)CCO2]

In some embodiments, R₅ is

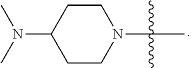 [SMILES: *N1CCC(N(C)C)CC1]

In some embodiments, R₅ is

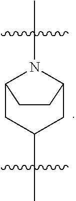 [SMILES: *C1CC2CCC(C1)N2*]

In some embodiments, R₅ is

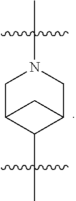 [SMILES: *C1C2CC1CN(*)C2]

In some embodiments, R₅ is

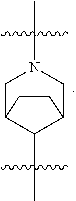 [SMILES: *C1C2CCC1CN(*)C2]

In some embodiments, R₅ is

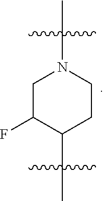 [SMILES: *C1CCN(*)CC1F]

In some embodiments, R₆ is absent or chosen from

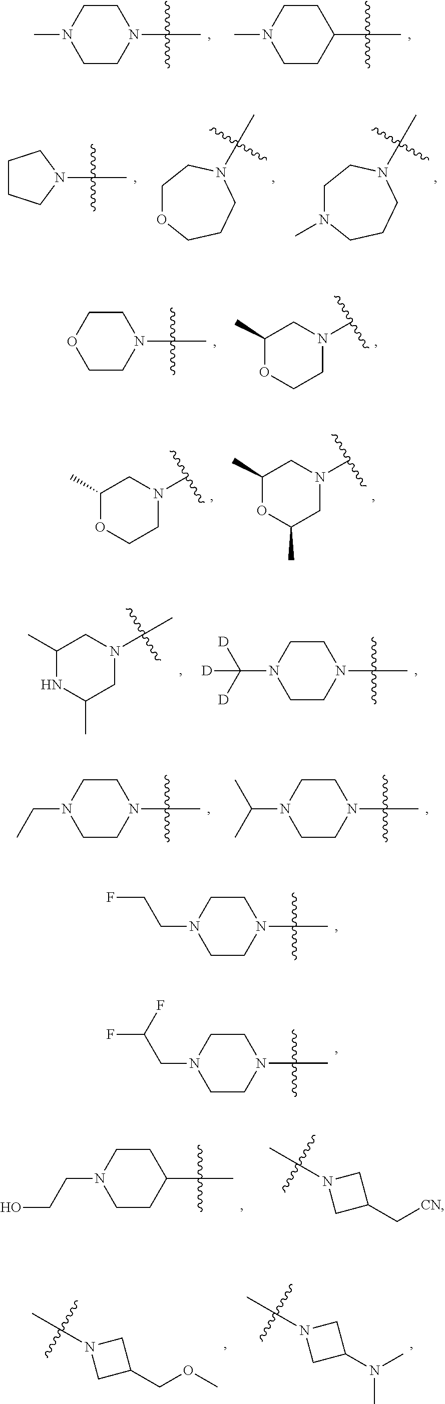

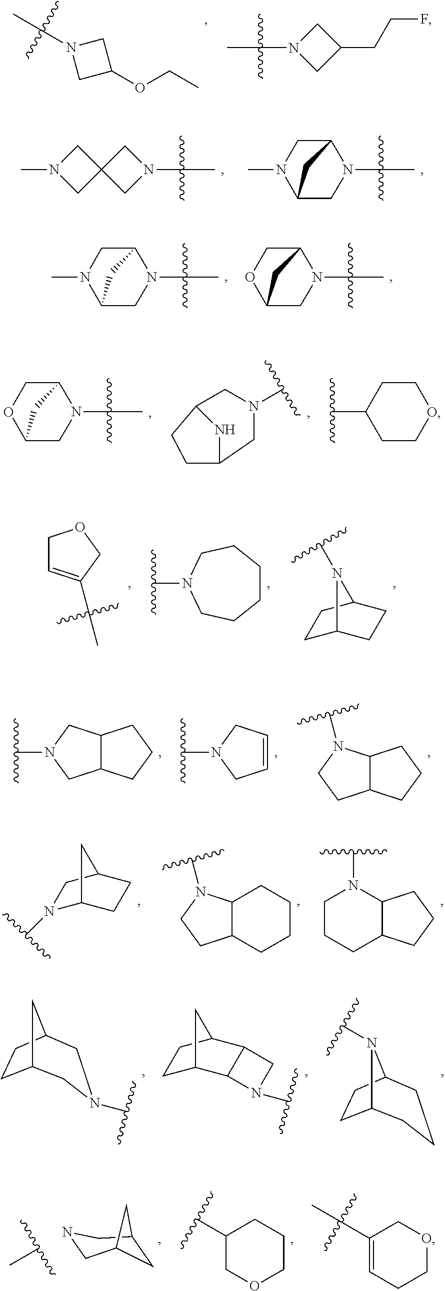

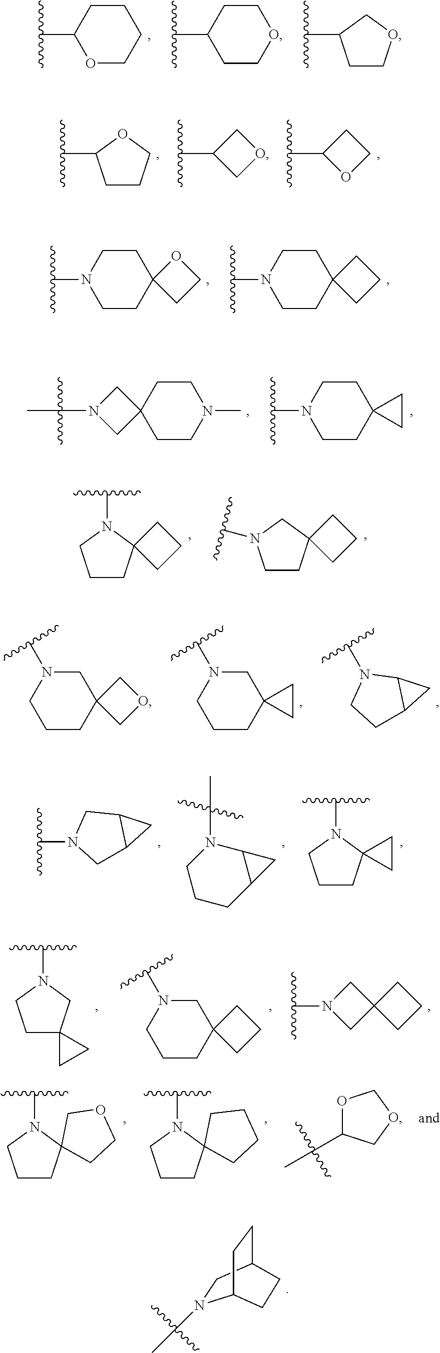

In some embodiments, R₆ is absent. In some embodiments, R₆ is

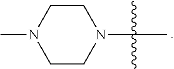 [SMILES: *N1CCN(C)CC1]

In some embodiments, R₆ is

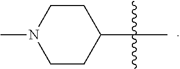 [SMILES: *C1CCN(C)CC1]

In some embodiments, R₆ is

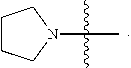 [SMILES: *N1CCCC1]

In some embodiments, R₆ is

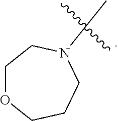 [SMILES: *N1CCCOCC1]

In some embodiments, R₆ is

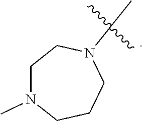 [SMILES: *N1CCCN(C)CC1]

In some embodiments, R₆ is

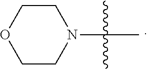 [SMILES: *N1CCOCC1]

In some embodiments, R₆ is

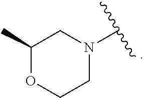 [SMILES: *N1CCO[C@@H](C)C1]

In some embodiments R₆ is

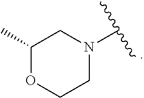 [SMILES: *N1CCO[C@H](C)C1]

In some embodiments, R₆ is

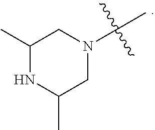 [SMILES: *N1CC(C)NC(C)C1]

In some embodiments, R₆ is

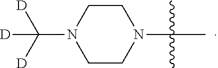 [SMILES: *N1CCN(C([2H])([2H])[2H])CC1]

In some embodiments, R₆ is

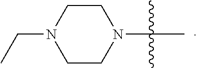 [SMILES: *N1CCN(CC)CC1]

In some embodiments, R₆ is

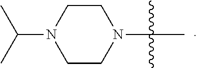 [SMILES: *N1CCN(C(C)C)CC1]

In some embodiments, R₆ is

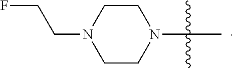 [SMILES: *N1CCN(CCF)CC1]

In some embodiments, R₆ is

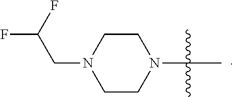 [SMILES: *N1CCN(CC(F)F)CC1]

In some embodiments, R₆ is

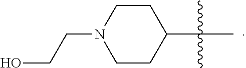 [SMILES: *C1CCN(CCO)CC1]

In some embodiments, R₆ is

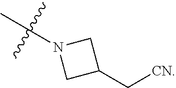 [SMILES: *N1CC(CC#N)C1]

In some embodiments, R₆ is

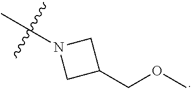 [SMILES: *N1CC(COC)C1]

In some embodiments, R₆ is

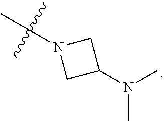 [SMILES: *N1CC(N(C)C)C1]

In some embodiments, R₆ is

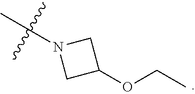 [SMILES: *N1CC(OCC)C1]

In some embodiments, R₆ is

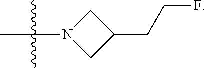 [SMILES: *N1CC(CCF)C1]

In some embodiments, R₆ is

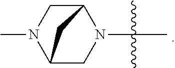 [SMILES: *N1C[C@H]2C[C@@H]1CN2C]

In some embodiments, R₆ is

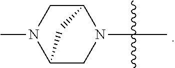 [SMILES: *N1C[C@@H]2C[C@H]1CN2C]

In some embodiments, R₆ is

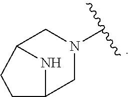 [SMILES: *N1CC2CCC(C1)N2]

In some embodiments, provided herein is a compound, or pharmaceutically acceptable salt thereof, chosen from the compounds listed in Table 1.

TABLE 1
Exemplary Compound of the Present Disclosure

| Compound | | |
|---|---|---|
| No. | Chemical Structure | IUPAC Nomenclature |
| 1 | 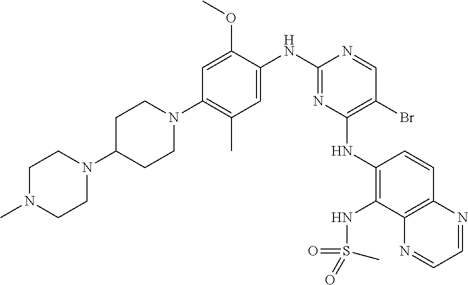 [SMILES: COc1cc(N2CCC(N3CCN(C)CC3)CC2)c(C)cc1Nc1ncc(Br)c(Nc2ccc3nccnc3c2NS(C)(=O)=O)n1] | N-(6-((5-bromo-2-((2-methoxy- 5-methyl-4-(4-(4- methylpiperazin-1-yl)piperidin- 1-yl)phenyl)amino)pyrimidin-4- yl)amino)quinoxalin-5- yl)methanesulfonamide |
| 2 | 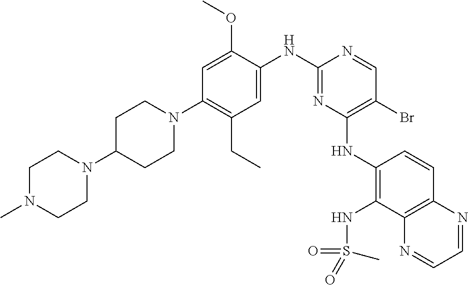 [SMILES: CCc1cc(Nc2ncc(Br)c(Nc3ccc4nccnc4c3NS(C)(=O)=O)n2)c(OC)cc1N1CCC(N2CCN(C)CC2)CC1] | N-(6-((5-bromo-2-((5-ethyl-2- methoxy-4-(4-(4- methylpiperazin-1-yl)piperidin- 1-yl)phenyl)amino)pyrimidin-4- yl)amino)quinoxalin-5- yl)methanesulfonamide |
| 3 | 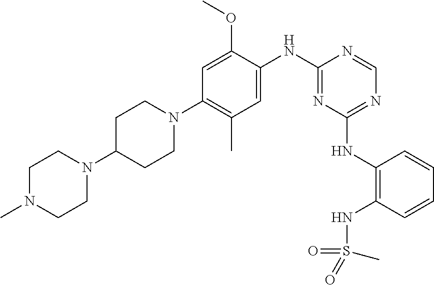 [SMILES: COc1cc(N2CCC(N3CCN(C)CC3)CC2)c(C)cc1Nc1ncnc(Nc2ccccc2NS(C)(=O)=O)n1] | N-(2-((4-((2-methoxy-5-methyl- 4-(4-(4-methylpiperazin-1- yl)piperidin-1-yl)phenyl)amino)- 1,3,5-triazin-2- yl)amino)phenyl)methanesulfon- amide |
| 4 | 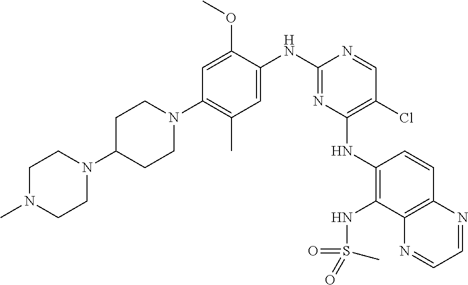 [SMILES: COc1cc(N2CCC(N3CCN(C)CC3)CC2)c(C)cc1Nc1ncc(Cl)c(Nc2ccc3nccnc3c2NS(C)(=O)=O)n1] | N-(6-((5-chloro-2-((2-methoxy- 5-methyl-4-(4-(4- methylpiperazin-1-yl)piperidin- 1-yl)phenyl)amino)pyrimidin-4- yl)amino)quinoxalin-5- yl)methanesulfonamide |
| 5 | 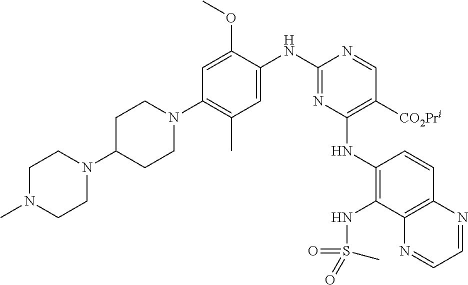 | isopropyl 2-((2-methoxy-5- methyl-4-(4-(4-methylpiperazin- 1-yl)piperidin-1- yl)phenyl)amino)-4-((5- (methylsulfonamido)quinoxalin- 6-yl)amino)pyrimidine-5- carboxylate |
| 6 | 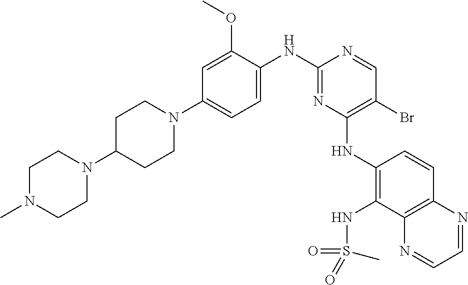 [SMILES: COc1cc(N2CCC(N3CCN(C)CC3)CC2)ccc1Nc1ncc(Br)c(Nc2ccc3nccnc3c2NS(C)(=O)=O)n1] | N-(6-((5-bromo-2-((2-methoxy- 4-(4-(4-methylpiperazin-1- yl)piperidin-1- yl)phenyl)amino)pyrimidin-4- yl)amino)quinoxalin-5- yl)methanesulfonamide |
| 7 | 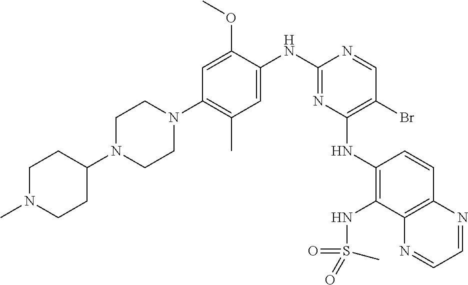 [SMILES: COc1cc(N2CCN(C3CCN(C)CC3)CC2)c(C)cc1Nc1ncc(Br)c(Nc2ccc3nccnc3c2NS(C)(=O)=O)n1] | N-(6-((5-bromo-2-((2-methoxy- 5-methyl-4-(4-(1- methylpiperidin-4-yl)piperazin- 1-yl)phenyl)amino)pyrimidin-4- yl)amino)quinoxalin-5- yl)methanesulfonamide |
| 8 | 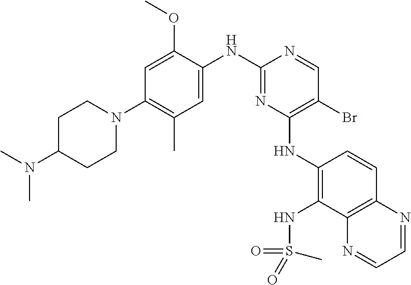 [SMILES: COc1cc(N2CCC(N(C)C)CC2)c(C)cc1Nc1ncc(Br)c(Nc2ccc3nccnc3c2NS(C)(=O)=O)n1] | N-(6-((5-bromo-2-((4-(4- (dimethylamino)piperidin-1-yl)- 2-methoxy-5- methylphenyl)amino)pyrimidin- 4-yl)amino)quinoxalin-5- yl)methanesulfonamide |
| 9 | 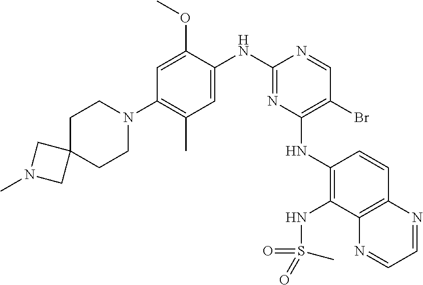 [SMILES: COc1cc(N2CCC3(CC2)CN(C)C3)c(C)cc1Nc1ncc(Br)c(Nc2ccc3nccnc3c2NS(C)(=O)=O)n1] | N-(6-((5-bromo-2-((2-methoxy- 5-methyl-4-(2-methyl-2,7- diazaspiro[3.5]nonan-7- yl)phenyl)amino)pyrimidin-4- yl)amino)quinoxalin-5- yl)methanesulfonamide |
| 10 | 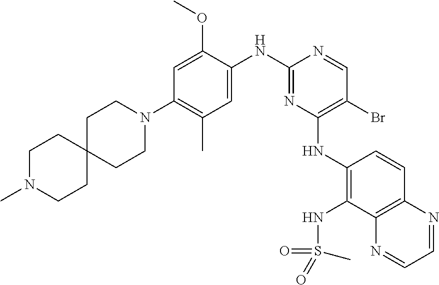 [SMILES: COc1cc(N2CCC3(CCN(C)CC3)CC2)c(C)cc1Nc1ncc(Br)c(Nc2ccc3nccnc3c2NS(C)(=O)=O)n1] | N-(6-((5-bromo-2-((2-methoxy- 5-methyl-4-(9-methyl-3,9- diazaspiro[5.5]undecan-3- yl)phenyl)amino)pyrimidin-4- yl)amino)quinoxalin-5- yl)methanesulfonamide |
| 11 | 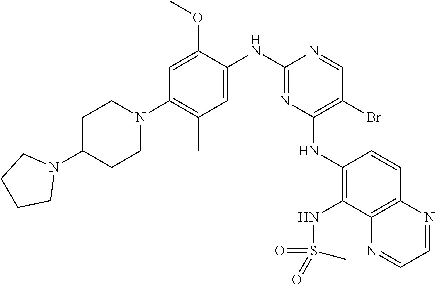 [SMILES: COc1cc(N2CCC(N3CCCC3)CC2)c(C)cc1Nc1ncc(Br)c(Nc2ccc3nccnc3c2NS(C)(=O)=O)n1] | N-(6-((5-bromo-2-((2-methoxy- 5-methyl-4-(4-(pyrrolidin-1- yl)piperidin-1- yl)phenyl)amino)pyrimidin-4- yl)amino)quinoxalin-5- yl)methanesulfonamide |
| 12 | 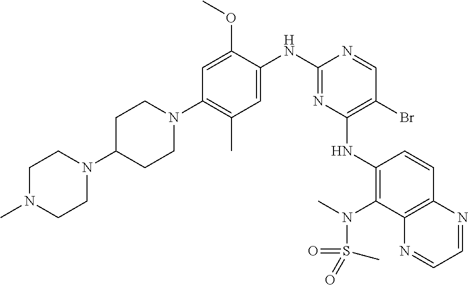 [SMILES: COc1cc(N2CCC(N3CCN(C)CC3)CC2)c(C)cc1Nc1ncc(Br)c(Nc2ccc3nccnc3c2N(C)S(C)(=O)=O)n1] | N-(6-((5-bromo-2-((2-methoxy- 5-methyl-4-(4-(4- methylpiperazin-1-yl)piperidin- 1-yl)phenyl)amino)pyrimidin-4- yl)amino)quinoxalin-5-yl)-N- methylmethanesulfonamide |
| 13 | 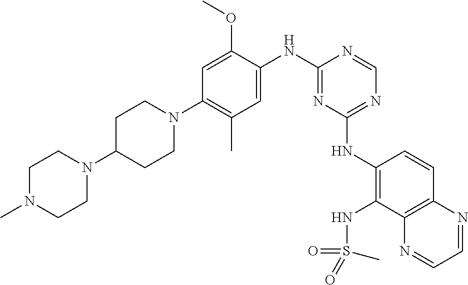 [SMILES: COc1cc(N2CCC(N3CCN(C)CC3)CC2)c(C)cc1Nc1ncnc(Nc2ccc3nccnc3c2NS(C)(=O)=O)n1] | N-(6-((4-((2-methoxy-5-methyl- 4-(4-(4-methylpiperazin-1- yl)piperidin-1-yl)phenyl)amino)- 1,3,5-triazin-2- yl)amino)quinoxalin-5- yl)methanesulfonamide |
| 14 | 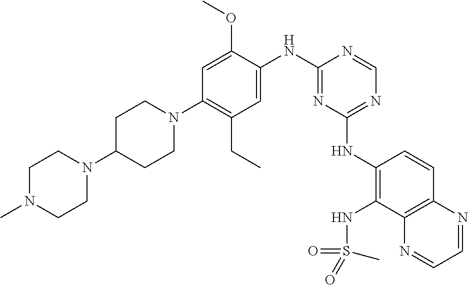 [SMILES: CCc1cc(Nc2ncnc(Nc3ccc4nccnc4c3NS(C)(=O)=O)n2)c(OC)cc1N1CCC(N2CCN(C)CC2)CC1] | N-(6-((4-((5-ethyl-2-methoxy-4- (4-(4-methylpiperazin-1- yl)piperidin-1-yl)phenyl)amino)- 1,3,5-triazin-2- yl)amino)quinoxalin-5- yl)methanesulfonamide |
| 15 | 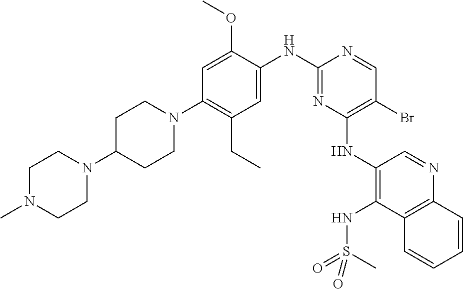 [SMILES: CCc1cc(Nc2ncc(Br)c(Nc3cnc4ccccc4c3NS(C)(=O)=O)n2)c(OC)cc1N1CCC(N2CCN(C)CC2)CC1] | N-(3-((5-bromo-2-((5-ethyl-2- methoxy-4-(4-(4- methylpiperazin-1-yl)piperidin- 1-yl)phenyl)amino)pyrimidin-4- yl)amino)quinolin-4- yl)methanesulfonamide |
| 16 | 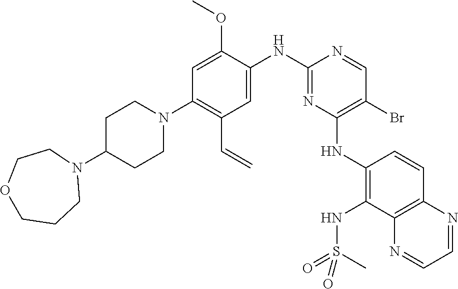 [SMILES: C=Cc1cc(Nc2ncc(Br)c(Nc3ccc4nccnc4c3NS(C)(=O)=O)n2)c(OC)cc1N1CCC(N2CCCOCC2)CC1] | N-(6-((2-((4-(4-(1,4-oxazepan- 4-yl)piperidin-1-yl)-2-methoxy- 5-vinylphenyl)amino)-5- bromopyrimidin-4- yl)amino)quinoxalin-5- yl)methanesulfonamide |
| 17 | 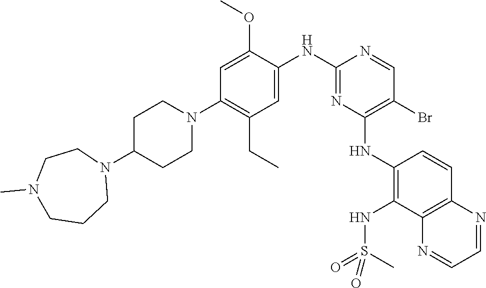 [SMILES: CCc1cc(Nc2ncc(Br)c(Nc3ccc4nccnc4c3NS(C)(=O)=O)n2)c(OC)cc1N1CCC(N2CCCN(C)CC2)CC1] | N-(6-((5-bromo-2-((5-ethyl-2- methoxy-4-(4-(4-methyl-1,4- diazepan-1-yl)piperidin-1- yl)phenyl)amino)pyrimidin-4- yl)amino)quinoxalin-5- yl)methanesulfonamide |
| 18 | 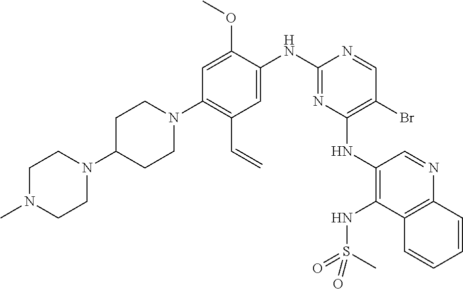 [SMILES: C=Cc1cc(Nc2ncc(Br)c(Nc3cnc4ccccc4c3NS(C)(=O)=O)n2)c(OC)cc1N1CCC(N2CCN(C)CC2)CC1] | N-(3-((5-bromo-2-((2-methoxy- 4-(4-(4-methylpiperazin-1- yl)piperidin-1-yl)-5- vinylphenyl)amino)pyrimidin-4- yl)amino)quinolin-4- yl)methanesulfonamide |
| 19 | 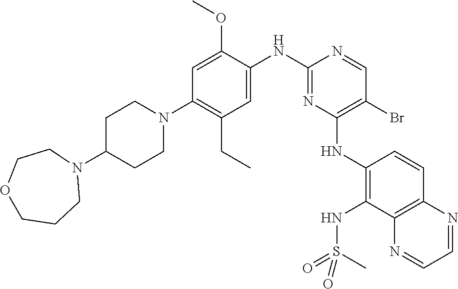 [SMILES: CCc1cc(Nc2ncc(Br)c(Nc3ccc4nccnc4c3NS(C)(=O)=O)n2)c(OC)cc1N1CCC(N2CCCOCC2)CC1] | N-(6-((2-((4-(4-(1,4-oxazepan- 4-yl)piperidin-1-yl)-5-ethyl-2- methoxyphenyl)amino)-5- bromopyrimidin-4- yl)amino)quinoxalin-5- yl)methanesulfonamide |
| 20 | 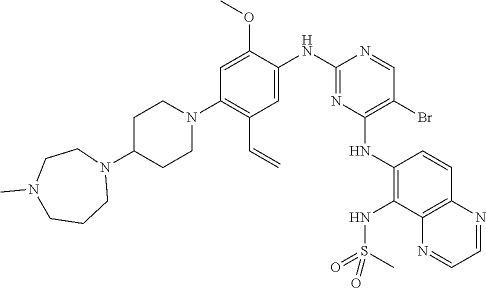 [SMILES: C=Cc1cc(Nc2ncc(Br)c(Nc3ccc4nccnc4c3NS(C)(=O)=O)n2)c(OC)cc1N1CCC(N2CCCN(C)CC2)CC1] | N-(6-((5-bromo-2-((2-methoxy- 4-(4-(4-methyl-1,4-diazepan-1- yl)piperidin-1-yl)-5- vinylphenyl)amino)pyrimidin-4- yl)amino)quinoxalin-5- yl)methanesulfonamide |
| 21 | 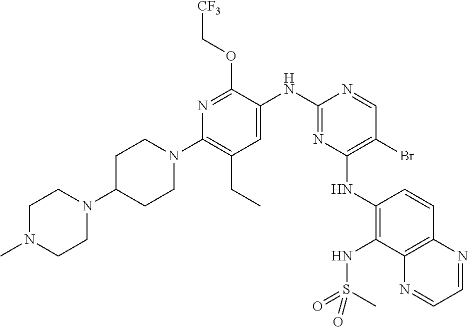 [SMILES: CCc1cc(Nc2ncc(Br)c(Nc3ccc4nccnc4c3NS(C)(=O)=O)n2)c(OCC(F)(F)F)nc1N1CCC(N2CCN(C)CC2)CC1] | N-(6-((5-bromo-2-((5-ethyl-6-(4- (4-methylpiperazin-1- yl)piperidin-1-yl)-2-(2,2,2- trifluoroethoxy)pyridin-3- yl)amino)pyrimidin-4- yl)amino)quinoxalin-5- yl)methanesulfonamide |
| 22 | 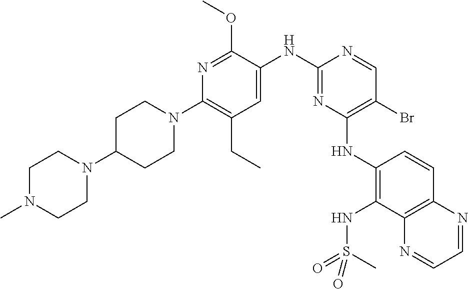 [SMILES: CCc1cc(Nc2ncc(Br)c(Nc3ccc4nccnc4c3NS(C)(=O)=O)n2)c(OC)nc1N1CCC(N2CCN(C)CC2)CC1] | N-(6-((5-bromo-2-((5-ethyl-2- methoxy-6-(4-(4- methylpiperazin-1-yl)piperidin- 1-yl)pyridin-3- yl)amino)pyrimidin-4- yl)amino)quinoxalin-5- yl)methanesulfonamide |
| 23 | 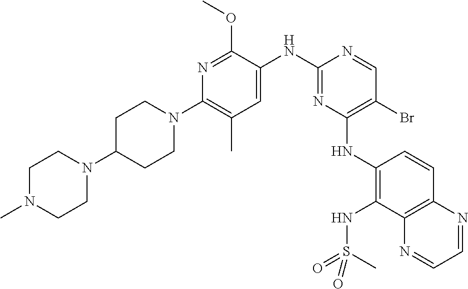 [SMILES: COc1nc(N2CCC(N3CCN(C)CC3)CC2)c(C)cc1Nc1ncc(Br)c(Nc2ccc3nccnc3c2NS(C)(=O)=O)n1] | N-(6-((5-bromo-2-((2-methoxy- 5-methyl-6-(4-(4- methylpiperazin-1-yl)piperidin- 1-yl)pyridin-3- yl)amino)pyrimidin-4- yl)amino)quinoxalin-5- yl)methanesulfonamide |
| 24 | 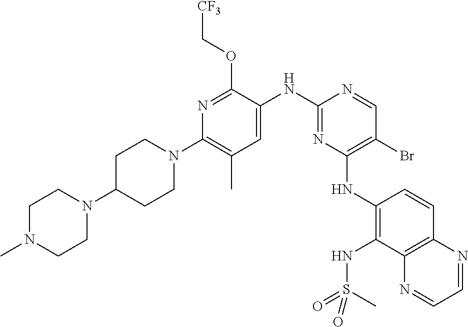 [SMILES: Cc1cc(Nc2ncc(Br)c(Nc3ccc4nccnc4c3NS(C)(=O)=O)n2)c(OCC(F)(F)F)nc1N1CCC(N2CCN(C)CC2)CC1] | N-(6-((5-bromo-2-((5-methyl-6- (4-(4-methylpiperazin-1- yl)piperidin-1-yl)-2-(2,2,2- trifluoroethoxy)pyridin-3- yl)amino)pyrimidin-4- yl)amino)quinoxalin-5- yl)methanesulfonamide |
| 25 | 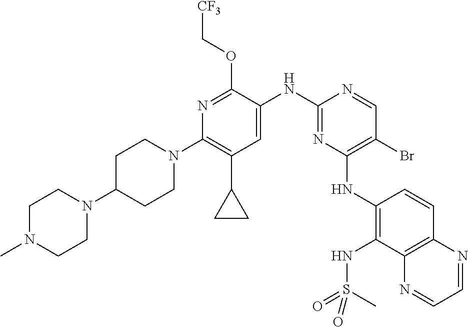 [SMILES: CN1CCN(C2CCN(c3nc(OCC(F)(F)F)c(Nc4ncc(Br)c(Nc5ccc6nccnc6c5NS(C)(=O)=O)n4)cc3C3CC3)CC2)CC1] | N-(6-((5-bromo-2-((5- cyclopropyl-6-(4-(4- methylpiperazin-1-yl)piperidin- 1-yl)-2-(2,2,2- trifluoroethoxy)pyridin-3- yl)amino)pyrimidin-4- yl)amino)quinoxalin-5- yl)methanesulfonamide |
| 26 | 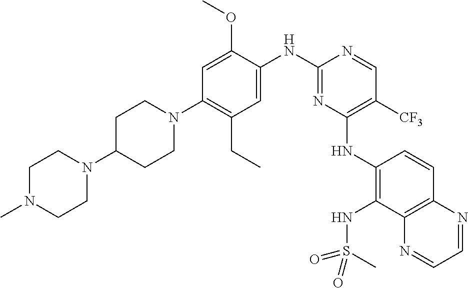 [SMILES: CCc1cc(Nc2ncc(C(F)(F)F)c(Nc3ccc4nccnc4c3NS(C)(=O)=O)n2)c(OC)cc1N1CCC(N2CCN(C)CC2)CC1] | N-(6-((2-((5-ethyl-2-methoxy-4- (4-(4-methylpiperazin-1- yl)piperidin-1-yl)phenyl)amino)- 5-(trifluoromethyl)pyrimidin-4- yl)amino)quinoxalin-5- yl)methanesulfonamide |
| 27 | 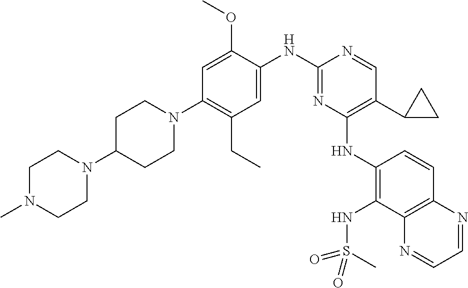 [SMILES: CCc1cc(Nc2ncc(C3CC3)c(Nc3ccc4nccnc4c3NS(C)(=O)=O)n2)c(OC)cc1N1CCC(N2CCN(C)CC2)CC1] | N-(6-((5-cyclopropyl-2-((5-ethyl- 2-methoxy-4-(4-(4- methylpiperazin-1-yl)piperidin- 1-yl)phenyl)amino)pyrimidin-4- yl)amino)quinoxalin-5- yl)methanesulfonamide |
| 28 | 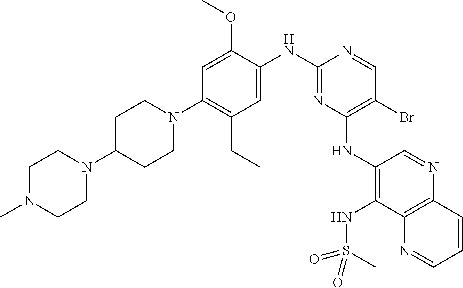 [SMILES: CCc1cc(Nc2ncc(Br)c(Nc3cnc4cccnc4c3NS(C)(=O)=O)n2)c(OC)cc1N1CCC(N2CCN(C)CC2)CC1] | N-(3-((5-bromo-2-((5-ethyl-2- methoxy-4-(4-(4- methylpiperazin-1-yl)piperidin- 1-yl)phenyl)amino)pyrimidin-4- yl)amino)-1,5-naphthyridin-4- yl)methanesulfonamide |
| 29 | 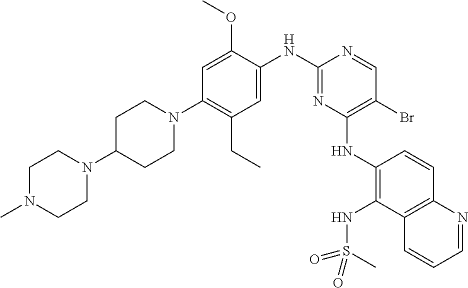 [SMILES: CCc1cc(Nc2ncc(Br)c(Nc3ccc4ncccc4c3NS(C)(=O)=O)n2)c(OC)cc1N1CCC(N2CCN(C)CC2)CC1] | N-(6-((5-bromo-2-((5-ethyl-2- methoxy-4-(4-(4- methylpiperazin-1-yl)piperidin- 1-yl)phenyl)amino)pyrimidin-4- yl)amino)quinolin-5- yl)methanesulfonamide |
| 30 | 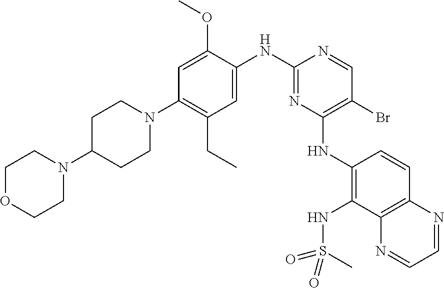 [SMILES: CCc1cc(Nc2ncc(Br)c(Nc3ccc4nccnc4c3NS(C)(=O)=O)n2)c(OC)cc1N1CCC(N2CCOCC2)CC1] | N-(6-((5-bromo-2-((5-ethyl-2- methoxy-4-(4- morpholinopiperidin-1- yl)phenyl)amino)pyrimidin-4- yl)amino)quinoxalin-5- yl)methanesulfonamide |
| 31 | 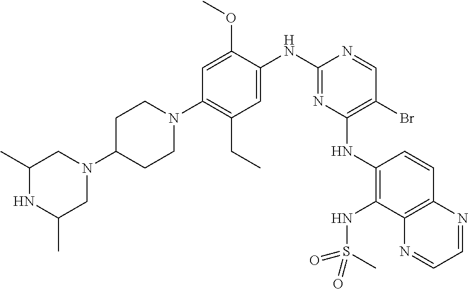 [SMILES: CCc1cc(Nc2ncc(Br)c(Nc3ccc4nccnc4c3NS(C)(=O)=O)n2)c(OC)cc1N1CCC(N2CC(C)NC(C)C2)CC1] | N-(6-((5-bromo-2-((4-(4-(3,5- dimethylpiperazin-1- yl)piperidin-1-yl)-5-ethyl-2- methoxyphenyl)amino)pyrimidin- 4-yl)amino)quinoxalin-5- yl)methanesulfonamide |
| 32 | 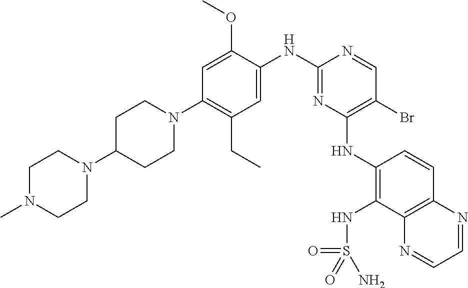 [SMILES: CCc1cc(Nc2ncc(Br)c(Nc3ccc4nccnc4c3NS(N)(=O)=O)n2)c(OC)cc1N1CCC(N2CCN(C)CC2)CC1] | N-(6-((5-bromo-2-((5-ethyl-2- methoxy-4-(4-(4- methylpiperazin-1-yl)piperidin- 1-yl)phenyl)amino)pyrimidin-4- yl)amino)quinoxalin-5- yl)sulfamide |
| 33 | 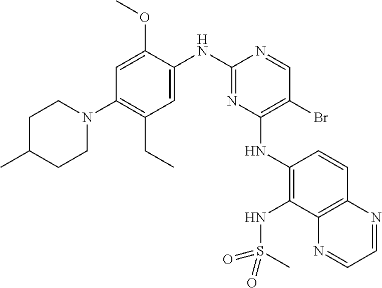 [SMILES: CCc1cc(Nc2ncc(Br)c(Nc3ccc4nccnc4c3NS(C)(=O)=O)n2)c(OC)cc1N1CCC(C)CC1] | N-(6-((5-bromo-2-((5-ethyl-2- methoxy-4-(4-methylpiperidin- 1-yl)phenyl)amino)pyrimidin-4- yl)amino)quinoxalin-5- yl)methanesulfonamide |
| 34 | 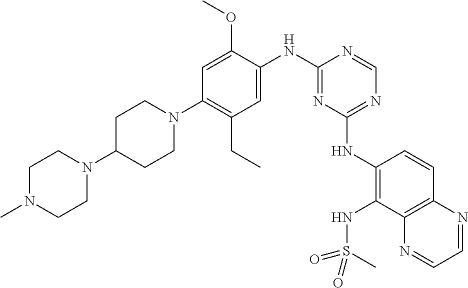 [SMILES: CCc1cc(Nc2ncnc(Nc3ccc4nccnc4c3NS(C)(=O)=O)n2)c(OC)cc1N1CCC(N2CCN(C)CC2)CC1] | N-(6-((4-((5-ethyl-2-methoxy-4- (4-(4-methylpiperazin-1- yl)piperidin-1-yl)phenyl)amino)- 1,3,5-triazin-2- yl)amino)quinoxalin-5- yl)methanesulfonamide |
| 35 | 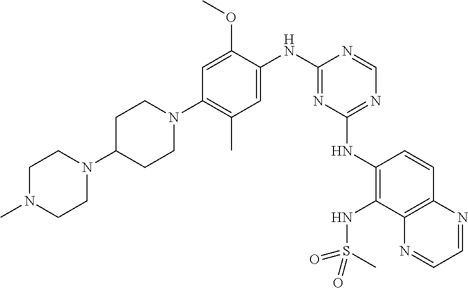 [SMILES: COc1cc(N2CCC(N3CCN(C)CC3)CC2)c(C)cc1Nc1ncnc(Nc2ccc3nccnc3c2NS(C)(=O)=O)n1] | N-(6-((4-((2-methoxy-5-methyl- 4-(4-(4-methylpiperazin-1- yl)piperidin-1-yl)phenyl)amino)- 1,3,5-triazin-2- yl)amino)quinoxalin-5- yl)methanesulfonamide |
| 36 | 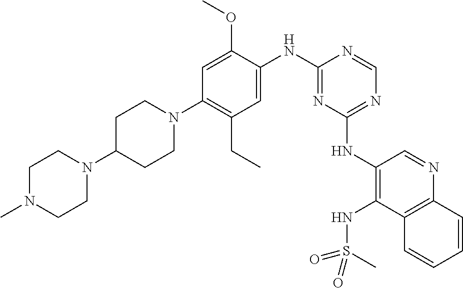 [SMILES: CCc1cc(Nc2ncnc(Nc3cnc4ccccc4c3NS(C)(=O)=O)n2)c(OC)cc1N1CCC(N2CCN(C)CC2)CC1] | N-(3-((4-((5-ethyl-2-methoxy-4- (4-(4-methylpiperazin-1- yl)piperidin-1-yl)phenyl)amino)- 1,3,5-triazin-2- yl)amino)quinolin-4- yl)methanesulfonamide |
| 37 | 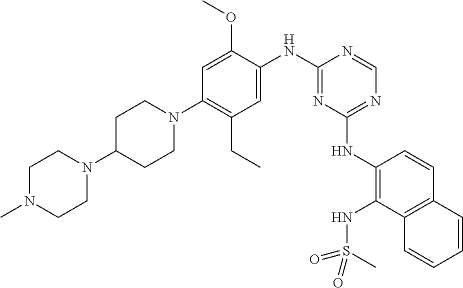 [SMILES: CCc1cc(Nc2ncnc(Nc3ccc4ccccc4c3NS(C)(=O)=O)n2)c(OC)cc1N1CCC(N2CCN(C)CC2)CC1] | N-(2-((4-((5-ethyl-2-methoxy-4- (4-(4-methylpiperazin-1- yl)piperidin-1-yl)phenyl)amino)- 1,3,5-triazin-2- yl)amino)naphthalen-1- yl)methanesulfonamide |
| 38 | 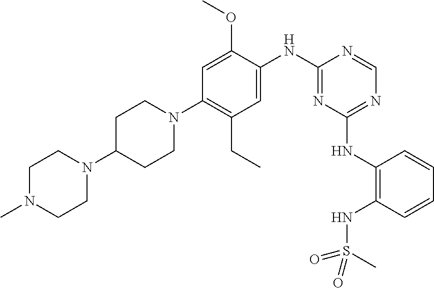 [SMILES: CCc1cc(Nc2ncnc(Nc3ccccc3NS(C)(=O)=O)n2)c(OC)cc1N1CCC(N2CCN(C)CC2)CC1] | N-(2-((4-((5-ethyl-2-methoxy-4- (4-(4-methylpiperazin-1- yl)piperidin-1-yl)phenyl)amino)- 1,3,5-triazin-2- yl)amino)phenyl)methanesulfon- amide |
| 39 | 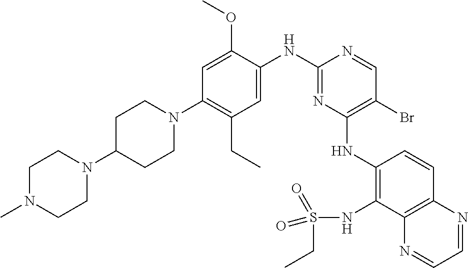 [SMILES: CCc1cc(Nc2ncc(Br)c(Nc3ccc4nccnc4c3NS(=O)(=O)CC)n2)c(OC)cc1N1CCC(N2CCN(C)CC2)CC1] | N-(6-((5-bromo-2-((5-ethyl-2- methoxy-4-(4-(4- methylpiperazin-1-yl)piperidin- 1-yl)phenyl)amino)pyrimidin-4- yl)amino)quinoxalin-5- yl)ethanesulfonamide |
| 40 | 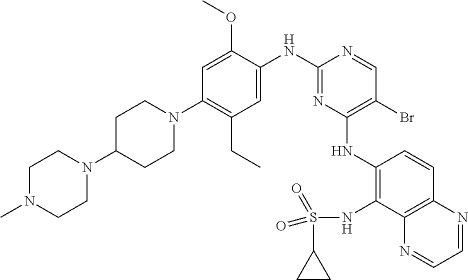 [SMILES: CCc1cc(Nc2ncc(Br)c(Nc3ccc4nccnc4c3NS(=O)(=O)C3CC3)n2)c(OC)cc1N1CCC(N2CCN(C)CC2)CC1] | N-(6-((5-bromo-2-((5-ethyl-2- methoxy-4-(4-(4- methylpiperazin-1-yl)piperidin- 1-yl)phenyl)amino)pyrimidin-4- yl)amino)quinoxalin-5- yl)cyclopropanesulfonamide |
| 41 | 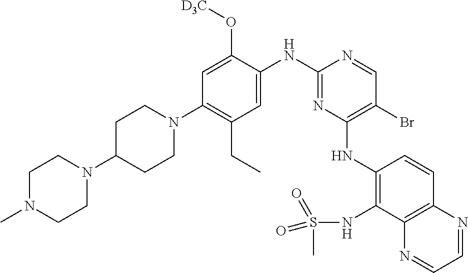 [SMILES: [2H]C([2H])([2H])Oc1cc(N2CCC(N3CCN(C)CC3)CC2)c(CC)cc1Nc1ncc(Br)c(Nc2ccc3nccnc3c2NS(C)(=O)=O)n1] | N-(6-((5-bromo-2-((5-ethyl-2- (methoxy-d3)-4-(4-(4- methylpiperazin-1-yl)piperidin- 1-yl)phenyl)amino)pyrimidin-4- yl)amino)quinoxalin-5- yl)methanesulfonamide |
| 42 | 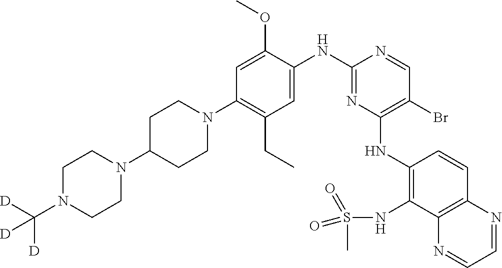 [SMILES: [2H]C([2H])([2H])N1CCN(C2CCN(c3cc(OC)c(Nc4ncc(Br)c(Nc5ccc6nccnc6c5NS(C)(=O)=O)n4)cc3CC)CC2)CC1] | N-(6-((5-bromo-2-((5-ethyl-2- methoxy-4-(4-(4-(methyl- d3)piperazin-1-yl)piperidin-1- yl)phenyl)amino)pyrimidin-4- yl)amino)quinoxalin-5- yl)methanesulfonamide |
| 43 | 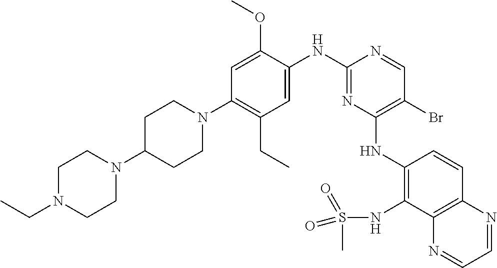 [SMILES: CCc1cc(Nc2ncc(Br)c(Nc3ccc4nccnc4c3NS(C)(=O)=O)n2)c(OC)cc1N1CCC(N2CCN(CC)CC2)CC1] | N-(6-((5-bromo-2-((5-ethyl-4-(4- (4-ethylpiperazin-1-yl)piperidin- 1-yl)-2- methoxyphenyl)amino)pyrimidin- 4-yl)amino)quinoxalin-5- yl)methanesulfonamide |
| 44 | 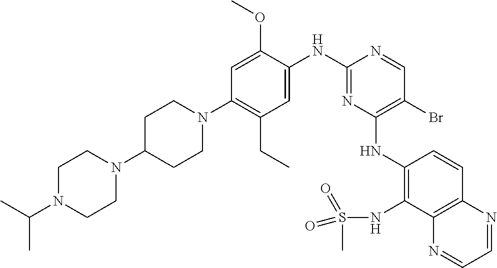 [SMILES: CCc1cc(Nc2ncc(Br)c(Nc3ccc4nccnc4c3NS(C)(=O)=O)n2)c(OC)cc1N1CCC(N2CCN(C(C)C)CC2)CC1] | N-(6-((5-bromo-2-((5-ethyl-4-(4- (4-isopropylpiperazin-1- yl)piperidin-1-yl)-2- methoxyphenyl)amino)pyrimidin- 4-yl)amino)quinoxalin-5- yl)methanesulfonamide |
| 45 | 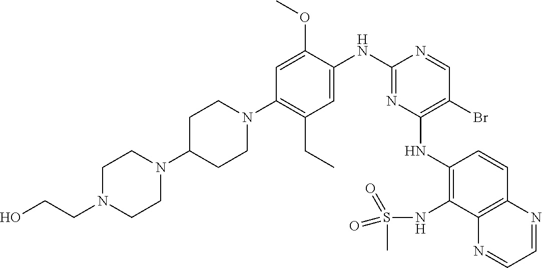 [SMILES: CCc1cc(Nc2ncc(Br)c(Nc3ccc4nccnc4c3NS(C)(=O)=O)n2)c(OC)cc1N1CCC(N2CCN(CCO)CC2)CC1] | N-(6-((5-bromo-2-((5-ethyl-4- (1′-(2-hydroxyethyl)-[1,4′- bipiperidin]-4-yl)-2- methoxyphenyl)amino)pyrimidin- 4-yl)amino)quinoxalin-5- yl)methanesulfonamide |
| 46 | 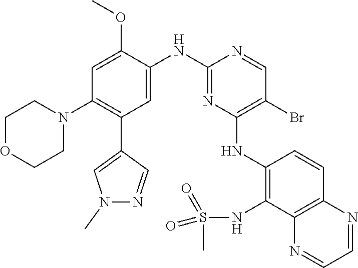 [SMILES: COc1cc(N2CCOCC2)c(-c2cnn(C)c2)cc1Nc1ncc(Br)c(Nc2ccc3nccnc3c2NS(C)(=O)=O)n1] | N-(6-((5-bromo-2-((2-methoxy- 5-(1-methyl-1H-pyrazol-4-yl)-4- morpholinophenyl)amino)pyrimi- din-4-yl)amino)quinoxalin-5- yl)methanesulfonamide |
| 47 | 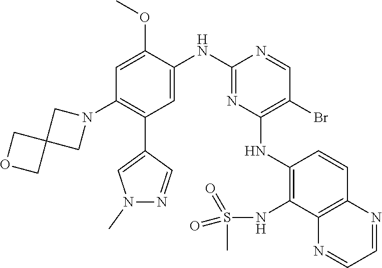 [SMILES: COc1cc(N2CC3(COC3)C2)c(-c2cnn(C)c2)cc1Nc1ncc(Br)c(Nc2ccc3nccnc3c2NS(C)(=O)=O)n1] | N-(6-((5-bromo-2-((2-methoxy- 5-(1-methyl-1H-pyrazol-4-yl)-4- (2-oxa-6-azaspiro[3.3]heptan-6- yl)phenyl)amino)pyrimidin-4- yl)amino)quinoxalin-5- yl)methanesulfonamide |
| 48 | 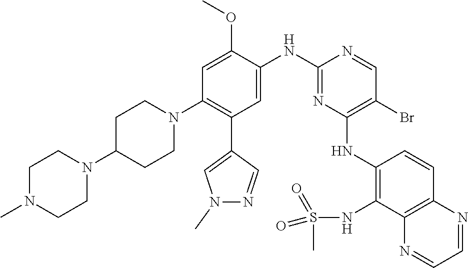 [SMILES: COc1cc(N2CCC(N3CCN(C)CC3)CC2)c(-c2cnn(C)c2)cc1Nc1ncc(Br)c(Nc2ccc3nccnc3c2NS(C)(=O)=O)n1] | N-(6-((5-bromo-2-((2-methoxy- 5-(1-methyl-1H-pyrazol-4-yl)-4- (4-(4-methylpiperazin-1- yl)piperidin-1- yl)phenyl)amino)pyrimidin-4- yl)amino)quinoxalin-5- yl)methanesulfonamide |
| 49 | 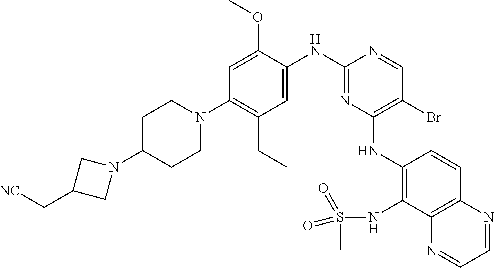 [SMILES: CCc1cc(Nc2ncc(Br)c(Nc3ccc4nccnc4c3NS(C)(=O)=O)n2)c(OC)cc1N1CCC(N2CC(CC#N)C2)CC1] | N-(6-((5-bromo-2-((4-(4-(3- (cyanomethyl)azetidin-1- yl)piperidin-1-yl)-5-ethyl-2- methoxyphenyl)amino)pyrimidin- 4-yl)amino)quinoxalin-5- yl)methanesulfonamide |
| 50 | 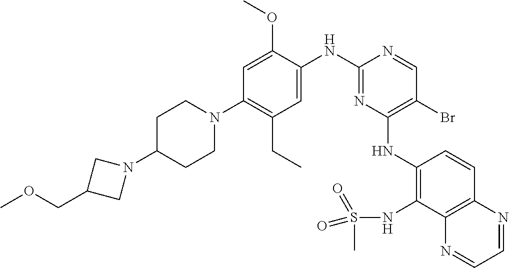 [SMILES: CCc1cc(Nc2ncc(Br)c(Nc3ccc4nccnc4c3NS(C)(=O)=O)n2)c(OC)cc1N1CCC(N2CC(COC)C2)CC1] | N-(6-((5-bromo-2-((5-ethyl-2- methoxy-4-(4-(3- (methoxymethyl)azetidin-1- yl)piperidin-1- yl)phenyl)amino)pyrimidin-4- yl)amino)quinoxalin-5- yl)methanesulfonamide |
| 51 | 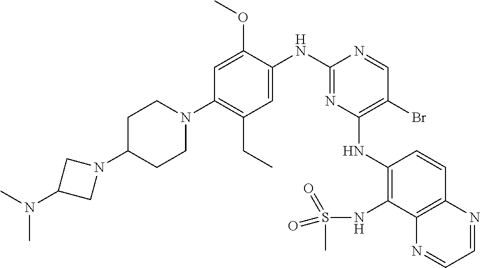 [SMILES: CCc1cc(Nc2ncc(Br)c(Nc3ccc4nccnc4c3NS(C)(=O)=O)n2)c(OC)cc1N1CCC(N2CC(N(C)C)C2)CC1] | N-(6-((5-bromo-2-((4-(4-(3- (dimethylamino)azetidin-1- yl)piperidin-1-yl)-5-ethyl-2- methoxyphenyl)amino)pyrimidin- 4-yl)amino)quinoxalin-5- yl)methanesulfonamide |
| 52 | 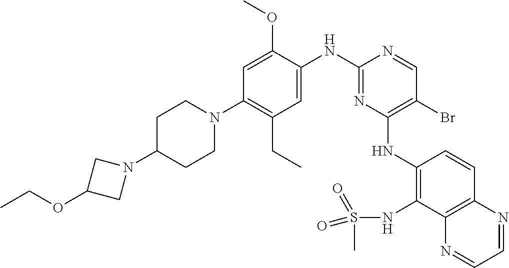 [SMILES: CCOC1CN(C2CCN(c3cc(OC)c(Nc4ncc(Br)c(Nc5ccc6nccnc6c5NS(C)(=O)=O)n4)cc3CC)CC2)C1] | N-(6-((5-bromo-2-((4-(4-(3- ethoxyazetidin-1-yl)piperidin-1- yl)-5-ethyl-2- methoxyphenyl)amino)pyrimidin- 4-yl)amino)quinoxalin-5- yl)methanesulfonamide |
| 53 | 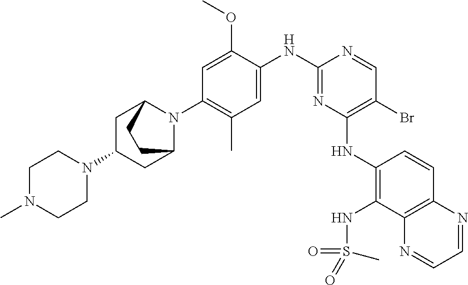 [SMILES: COc1cc(N2[C@@H]3CC[C@H]2C[C@H](N2CCN(C)CC2)C3)c(C)cc1Nc1ncc(Br)c(Nc2ccc3nccnc3c2NS(C)(=O)=O)n1] | N-(6-((5-bromo-2-((2-methoxy- 5-methyl-4-((1R,3s,5S)-3-(4- methylpiperazin-1-yl)-8- azabicyclo[3.2.1]octan-8- yl)phenyl)amino)pyrimidin-4- yl)amino)quinoxalin-5- yl)methanesulfonamide |
| 54 | 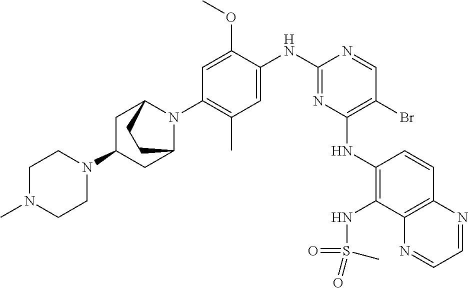 [SMILES: COc1cc(N2[C@@H]3CC[C@H]2C[C@@H](N2CCN(C)CC2)C3)c(C)cc1Nc1ncc(Br)c(Nc2ccc3nccnc3c2NS(C)(=O)=O)n1] | N-(6-((5-bromo-2-((2-methoxy- 5-methyl-4-((1R,3r,5S)-3-(4- methylpiperazin-1-yl)-8- azabicyclo[3.2.1]octan-8- yl)phenyl)amino)pyrimidin-4- yl)amino)quinoxalin-5- yl)methanesulfonamide |
| 55 | 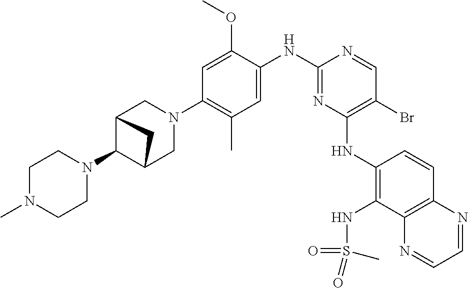 [SMILES: COc1cc(N2C[C@H]3C[C@@H](C2)[C@@H]3N2CCN(C)CC2)c(C)cc1Nc1ncc(Br)c(Nc2ccc3nccnc3c2NS(C)(=O)=O)n1] | N-(6-((5-bromo-2-((2-methoxy- 5-methyl-4-((1R,5S,6s)-6-(4- methylpiperazin-1-yl)-3- azabicyclo[3.1.1]heptan-3- yl)phenyl)amino)pyrimidin-4- yl)amino)quinoxalin-5- yl)methanesulfonamide |
| 56 | 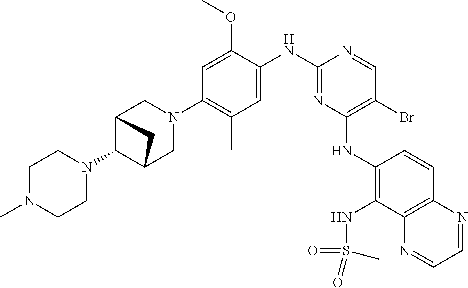 [SMILES: COc1cc(N2C[C@H]3C[C@@H](C2)[C@H]3N2CCN(C)CC2)c(C)cc1Nc1ncc(Br)c(Nc2ccc3nccnc3c2NS(C)(=O)=O)n1] | N-(6-((5-bromo-2-((2-methoxy- 5-methyl-4-((1R,5S,6r)-6-(4- methylpiperazin-1-yl)-3- azabicyclo[3.1.1]heptan-3- yl)phenyl)amino)pyrimidin-4- yl)amino)quinoxalin-5- yl)methanesulfonamide |
| 57 | 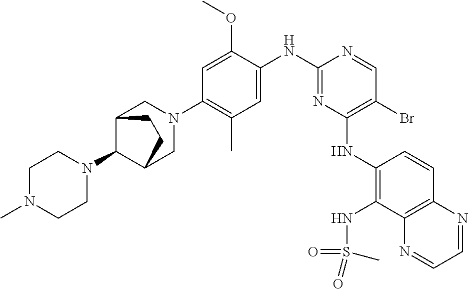 [SMILES: COc1cc(N2C[C@H]3CC[C@@H](C2)[C@@H]3N2CCN(C)CC2)c(C)cc1Nc1ncc(Br)c(Nc2ccc3nccnc3c2NS(C)(=O)=O)n1] | N-(6-((5-bromo-2-((2-methoxy- 5-methyl-4-((1R,5S,8s)-8-(4- methylpiperazin-1-yl)-3- azabicyclo[3.2.1]octan-3- yl)phenyl)amino)pyrimidin-4- yl)amino)quinoxalin-5- yl)methanesulfonamide |
| 58 | 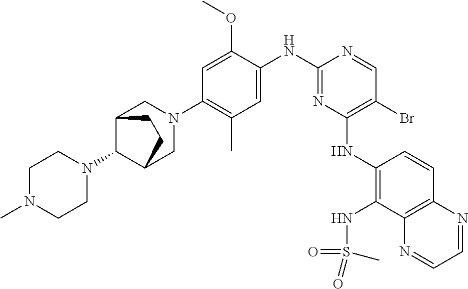 [SMILES: COc1cc(N2C[C@H]3CC[C@@H](C2)[C@H]3N2CCN(C)CC2)c(C)cc1Nc1ncc(Br)c(Nc2ccc3nccnc3c2NS(C)(=O)=O)n1] | N-(6-((5-bromo-2-((2-methoxy- 5-methyl-4-((1R,5S,8r)-8-(4- methylpiperazin-1-yl)-3- azabicyclo[3.2.1]octan-3- yl)phenyl)amino)pyrimidin-4- yl)amino)quinoxalin-5- yl)methanesulfonamide |
| 59 | 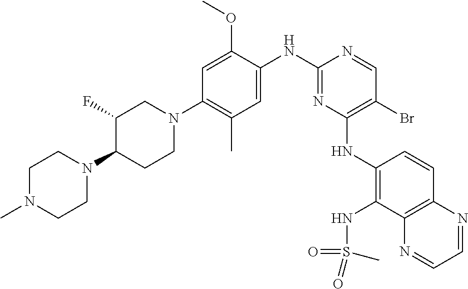 [SMILES: COc1cc(N2CC[C@@H](N3CCN(C)CC3)[C@H](F)C2)c(C)cc1Nc1ncc(Br)c(Nc2ccc3nccnc3c2NS(C)(=O)=O)n1] | N-(6-((5-bromo-2-((4-((3R,4R)- 3-fluoro-4-(4-methylpiperazin-1- yl)piperidin-1-yl)-2-methoxy-5- methylphenyl)amino)pyrimidin- 4-yl)amino)quinoxalin-5- yl)methanesulfonamide |
| 60 | 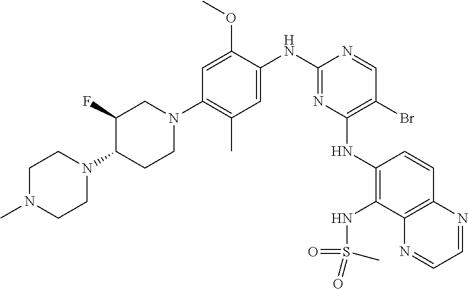 [SMILES: COc1cc(N2CC[C@H](N3CCN(C)CC3)[C@@H](F)C2)c(C)cc1Nc1ncc(Br)c(Nc2ccc3nccnc3c2NS(C)(=O)=O)n1] | N-(6-((5-bromo-2-((4-((3S,4S)- 3-fluoro-4-(4-methylpiperazin-1- yl)piperidin-1-yl)-2-methoxy-5- methylphenyl)amino)pyrimidin- 4-yl)amino)quinoxalin-5- yl)methanesulfonamide |
| 61 | 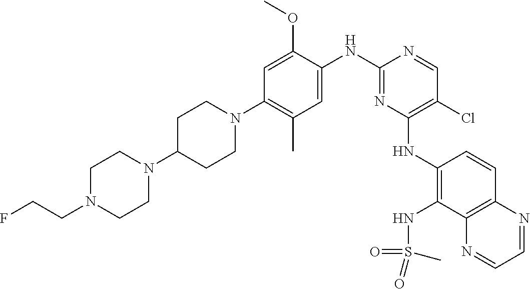 [SMILES: COc1cc(N2CCC(N3CCN(CCF)CC3)CC2)c(C)cc1Nc1ncc(Cl)c(Nc2ccc3nccnc3c2NS(C)(=O)=O)n1] | N-(6-((5-chloro-2-((4-(4-(4-(2- fluoroethyl)piperazin-1- yl)piperidin-1-yl)-2-methoxy-5- methylphenyl)amino)pyrimidin- 4-yl)amino)quinoxalin-5- yl)methanesulfonamide |
| 62 | 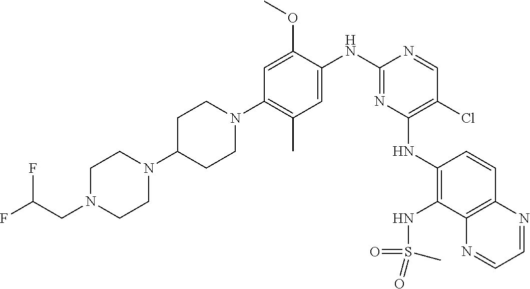 [SMILES: COc1cc(N2CCC(N3CCN(CC(F)F)CC3)CC2)c(C)cc1Nc1ncc(Cl)c(Nc2ccc3nccnc3c2NS(C)(=O)=O)n1] | N-(6-((5-chloro-2-((4-(4-(4-(2,2- difluoroethyl)piperazin-1- yl)piperidin-1-yl)-2-methoxy-5- methylphenyl)amino)pyrimidin- 4-yl)amino)quinoxalin-5- yl)methanesulfonamide |
| 63 | 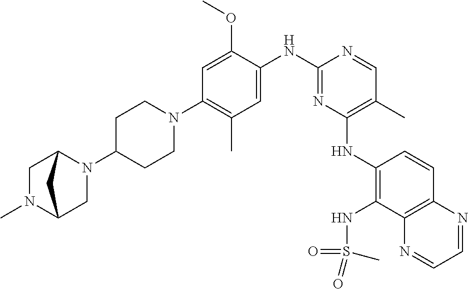 [SMILES: COc1cc(N2CCC(N3C[C@H]4C[C@@H]3CN4C)CC2)c(C)cc1Nc1ncc(C)c(Nc2ccc3nccnc3c2NS(C)(=O)=O)n1] | N-(6-((2-((2-methoxy-5-methyl- 4-(4-((1R,4R)-5-methyl-2,5- diazabicyclo[2.2.1]heptan-2- yl)piperidin-1-yl)phenyl)amino)- 5-methylpyrimidin-4- yl)amino)quinoxalin-5- yl)methanesulfonamide |
| 64 | 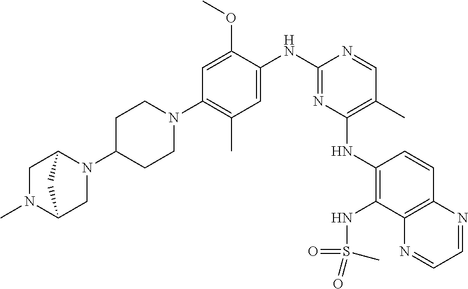 [SMILES: COc1cc(N2CCC(N3C[C@@H]4C[C@H]3CN4C)CC2)c(C)cc1Nc1ncc(C)c(Nc2ccc3nccnc3c2NS(C)(=O)=O)n1] | N-(6-((2-((2-methoxy-5-methyl- 4-(4-((1S,4S)-5-methyl-2,5- diazabicyclo[2.2.1]heptan-2- yl)piperidin-1-yl)phenyl)amino)- 5-methylpyrimidin-4- yl)amino)quinoxalin-5- yl)methanesulfonamide |
| 65 | 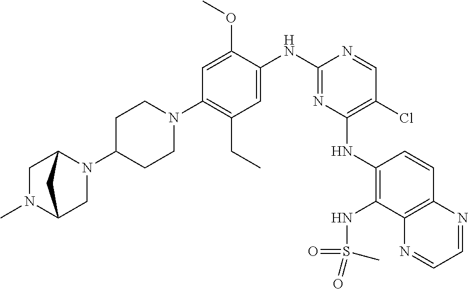 [SMILES: CCc1cc(Nc2ncc(Cl)c(Nc3ccc4nccnc4c3NS(C)(=O)=O)n2)c(OC)cc1N1CCC(N2C[C@H]3C[C@@H]2CN3C)CC1] | N-(6-((5-chloro-2-((5-ethyl-2- methoxy-4-(4-((1R,4R)-5- methyl-2,5- diazabicyclo[2.2.1]heptan-2- yl)piperidin-1- yl)phenyl)amino)pyrimidin-4- yl)amino)quinoxalin-5- yl)methanesulfonamide |
| 66 | 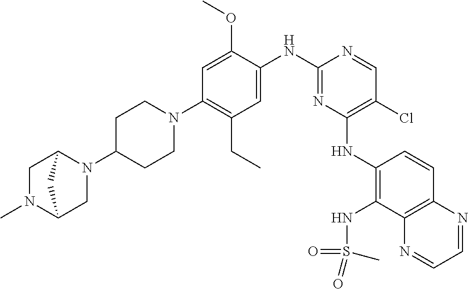 [SMILES: CCc1cc(Nc2ncc(Cl)c(Nc3ccc4nccnc4c3NS(C)(=O)=O)n2)c(OC)cc1N1CCC(N2C[C@@H]3C[C@H]2CN3C)CC1] | N-(6-((5-chloro-2-((5-ethyl-2- methoxy-4-(4-((1S,4S)-5- methyl-2,5- diazabicyclo[2.2.1]heptan-2- yl)piperidin-1- yl)phenyl)amino)pyrimidin-4- yl)amino)quinoxalin-5- yl)methanesulfonamide |
| 67 | 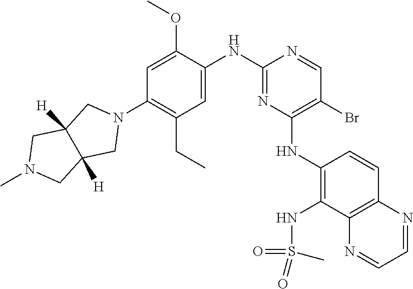 [SMILES: CCc1cc(Nc2ncc(Br)c(Nc3ccc4nccnc4c3NS(C)(=O)=O)n2)c(OC)cc1N1C[C@H]2CN(C)C[C@H]2C1] | N-(6-((5-bromo-2-((5-ethyl-2- methoxy-4-((3aR,6aS)-5- methylhexahydropyrrolo[3,4- c]pyrrol-2(1H)- yl)phenyl)amino)pyrimidin-4- yl)amino)quinoxalin-5- yl)methanesulfonamide |
| 68 | 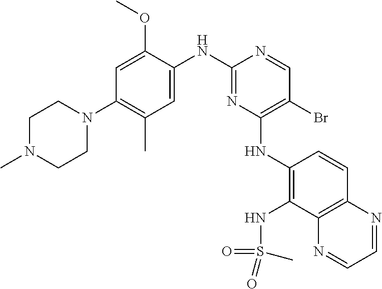 [SMILES: COc1cc(N2CCN(C)CC2)c(C)cc1Nc1ncc(Br)c(Nc2ccc3nccnc3c2NS(C)(=O)=O)n1] | N-(6-((5-bromo-2-((2-methoxy- 5-methyl-4-(4-methylpiperazin- 1-yl)phenyl)amino)pyrimidin-4- yl)amino)quinoxalin-5- yl)methanesulfonamide |
| 69 | 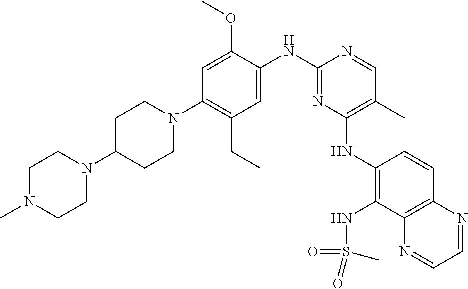 [SMILES: CCc1cc(Nc2ncc(C)c(Nc3ccc4nccnc4c3NS(C)(=O)=O)n2)c(OC)cc1N1CCC(N2CCN(C)CC2)CC1] | N-(6-((2-((5-ethyl-2-methoxy-4- (4-(4-methylpiperazin-1- yl)piperidin-1-yl)phenyl)amino)- 5-methylpyrimidin-4- yl)amino)quinoxalin-5- yl)methanesulfonamide |
| 70 | 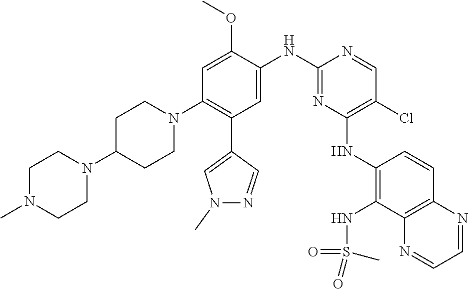 [SMILES: COc1cc(N2CCC(N3CCN(C)CC3)CC2)c(-c2cnn(C)c2)cc1Nc1ncc(Cl)c(Nc2ccc3nccnc3c2NS(C)(=O)=O)n1] | N-(6-((5-chloro-2-((2-methoxy- 5-(1-methyl-1H-pyrazol-4-yl)-4- (4-(4-methylpiperazin-1- yl)piperidin-1- yl)phenyl)amino)pyrimidin-4- yl)amino)quinoxalin-5- yl)methanesulfonamide |
| 71 | 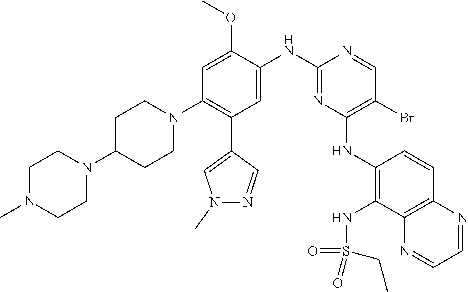 [SMILES: CCS(=O)(=O)Nc1c(Nc2nc(Nc3cc(-c4cnn(C)c4)c(N4CCC(N5CCN(C)CC5)CC4)cc3OC)ncc2Br)ccc2nccnc12] | N-(6-((5-bromo-2-((2-methoxy- 5-(1-methyl-1H-pyrazol-4-yl)-4- (4-(4-methylpiperazin-1- yl)piperidin-1- yl)phenyl)amino)pyrimidin-4- yl)amino)quinoxalin-5- yl)ethanesulfonamide |
| 72 | 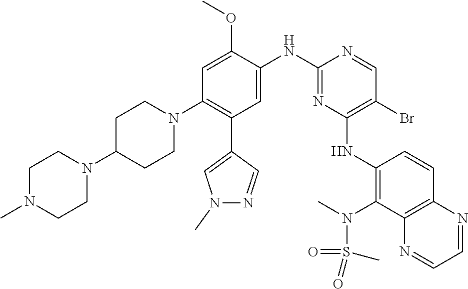 [SMILES: COc1cc(N2CCC(N3CCN(C)CC3)CC2)c(-c2cnn(C)c2)cc1Nc1ncc(Br)c(Nc2ccc3nccnc3c2N(C)S(C)(=O)=O)n1] | N-(6-((5-bromo-2-((2-methoxy- 5-(1-methyl-1H-pyrazol-4-yl)-4- (4-(4-methylpiperazin-1- yl)piperidin-1- yl)phenyl)amino)pyrimidin-4- yl)amino)quinoxalin-5-yl)-N- methylmethanesulfonamide |
| 73 | 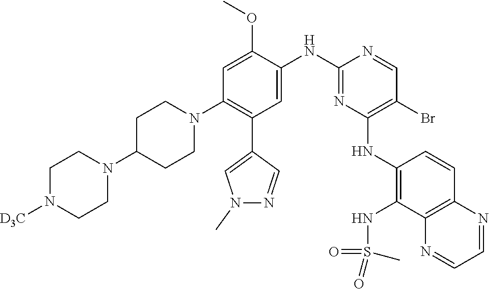 [SMILES: [2H]C([2H])([2H])N1CCN(C2CCN(c3cc(OC)c(Nc4ncc(Br)c(Nc5ccc6nccnc6c5NS(C)(=O)=O)n4)cc3-c3cnn(C)c3)CC2)CC1] | N-(6-((5-bromo-2-((2-methoxy- 5-(1-methyl-1H-pyrazol-4-yl)-4- (4-(4-(methyl-d3)piperazin-1- yl)piperidin-1- yl)phenyl)amino)pyrimidin-4- yl)amino)quinoxalin-5- yl)methanesulfonamide |
| 74 | 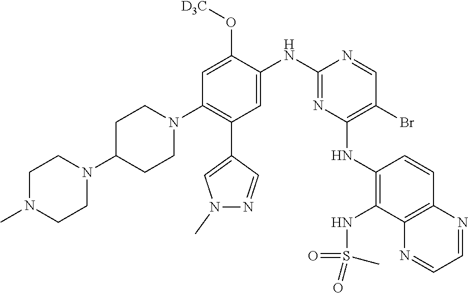 [SMILES: [2H]C([2H])([2H])Oc1cc(N2CCC(N3CCN(C)CC3)CC2)c(-c2cnn(C)c2)cc1Nc1ncc(Br)c(Nc2ccc3nccnc3c2NS(C)(=O)=O)n1] | N-(6-((5-bromo-2-((2-(methoxy- d3)-5-(1-methyl-1H-pyrazol-4- yl)-4-(4-(4-methylpiperazin-1- yl)piperidin-1- yl)phenyl)amino)pyrimidin-4- yl)amino)quinoxalin-5- yl)methanesulfonamide |
| 75 | 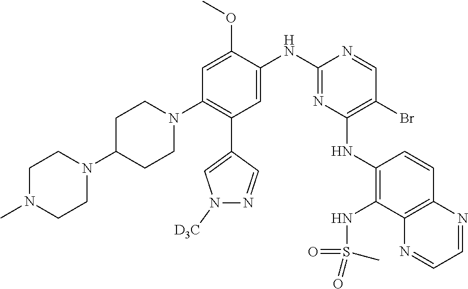 [SMILES: [2H]C([2H])([2H])n1cc(-c2cc(Nc3ncc(Br)c(Nc4ccc5nccnc5c4NS(C)(=O)=O)n3)c(OC)cc2N2CCC(N3CCN(C)CC3)CC2)cn1] | N-(6-((5-bromo-2-((2-methoxy- 5-(1-(methyl-d3)-1H-pyrazol-4- yl)-4-(4-(4-methylpiperazin-1- yl)piperidin-1- yl)phenyl)amino)pyrimidin-4- yl)amino)quinoxalin-5- yl)methanesulfonamide |
| 76 | 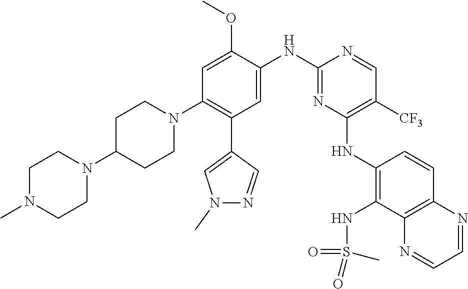 [SMILES: COc1cc(N2CCC(N3CCN(C)CC3)CC2)c(-c2cnn(C)c2)cc1Nc1ncc(C(F)(F)F)c(Nc2ccc3nccnc3c2NS(C)(=O)=O)n1] | N-(6-((2-((2-methoxy-5-(1- methyl-1H-pyrazol-4-yl)-4-(4-(4- methylpiperazin-1-yl)piperidin- 1-yl)phenyl)amino)-5- (trifluoromethyl)pyrimidin-4- yl)amino)quinoxalin-5- yl)methanesulfonamide |
| 77 | 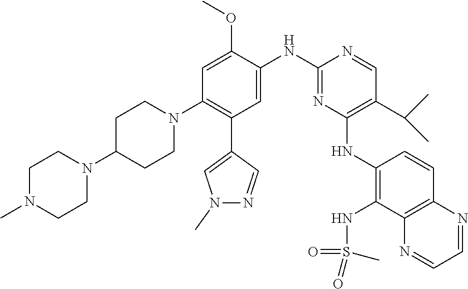 [SMILES: COc1cc(N2CCC(N3CCN(C)CC3)CC2)c(-c2cnn(C)c2)cc1Nc1ncc(C(C)C)c(Nc2ccc3nccnc3c2NS(C)(=O)=O)n1] | N-(6-((5-isopropyl-2-((2- methoxy-5-(1-methyl-1H- pyrazol-4-yl)-4-(4-(4- methylpiperazin-1-yl)piperidin- 1-yl)phenyl)amino)pyrimidin-4- yl)amino)quinoxalin-5- yl)methanesulfonamide |
| 78 | 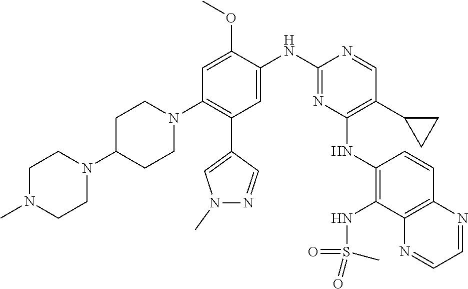 [SMILES: COc1cc(N2CCC(N3CCN(C)CC3)CC2)c(-c2cnn(C)c2)cc1Nc1ncc(C2CC2)c(Nc2ccc3nccnc3c2NS(C)(=O)=O)n1] | N-(6-((5-cyclopropyl-2-((2- methoxy-5-(1-methyl-1H- pyrazol-4-yl)-4-(4-(4- methylpiperazin-1-yl)piperidin- 1-yl)phenyl)amino)pyrimidin-4- yl)amino)quinoxalin-5- yl)methanesulfonamide |
| 79 | 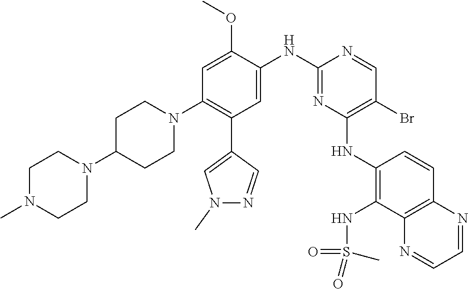 [SMILES: COc1cc(N2CCC(N3CCN(C)CC3)CC2)c(-c2cnn(C)c2)cc1Nc1ncc(Br)c(Nc2ccc3nccnc3c2NS(C)(=O)=O)n1] | N-(6-((5-bromo-2-((2-methoxy- 5-(1-methyl-1H-pyrazol-4-yl)-6- (4-(4-methylpiperazin-1- yl)piperidin-1-yl)pyridin-3- yl)amino)pyrimidin-4- yl)amino)quinoxalin-5- yl)methanesulfonamide |
| 80 | 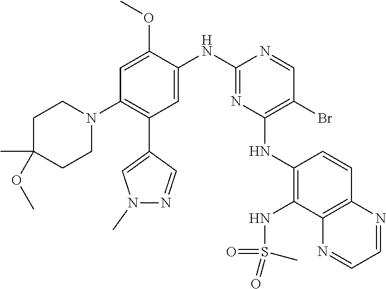 [SMILES: COc1cc(N2CCC(C)(OC)CC2)c(-c2cnn(C)c2)cc1Nc1ncc(Br)c(Nc2ccc3nccnc3c2NS(C)(=O)=O)n1] | N-(6-((5-bromo-2-((2-methoxy- 4-(4-methoxy-4- methylpiperidin-1-yl)-5-(1- methyl-1H-pyrazol-4- yl)phenyl)amino)pyrimidin-4- yl)amino)quinoxalin-5- yl)methanesulfonamide |
| 81 | 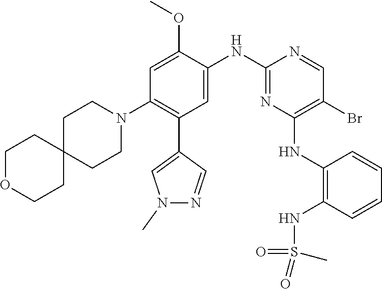 [SMILES: COc1cc(N2CCC3(CCOCC3)CC2)c(-c2cnn(C)c2)cc1Nc1ncc(Br)c(Nc2ccccc2NS(C)(=O)=O)n1] | N-(2-((5-bromo-2-((2-methoxy- 5-(1-methyl-1H-pyrazol-4-yl)-4- (3-oxa-9-azaspiro[5.5]undecan- 9-yl)phenyl)amino)pyrimidin-4- yl)amino)phenyl)methanesulfon- amide |
| 82 | 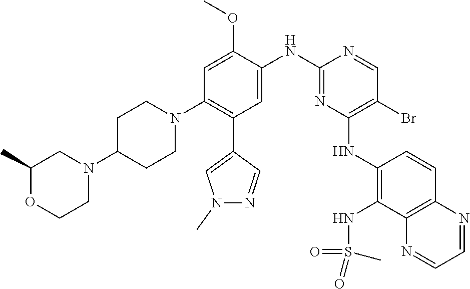 [SMILES: COc1cc(N2CCC(N3CCO[C@@H](C)C3)CC2)c(-c2cnn(C)c2)cc1Nc1ncc(Br)c(Nc2ccc3nccnc3c2NS(C)(=O)=O)n1] | (S)-N-(6-((5-bromo-2-((2- methoxy-5-(1-methyl-1H- pyrazol-4-yl)-4-(4-(2- methylmorpholino)piperidin-1- yl)phenyl)amino)pyrimidin-4- yl)amino)quinoxalin-5- yl)methanesulfonamide |
| 83 | 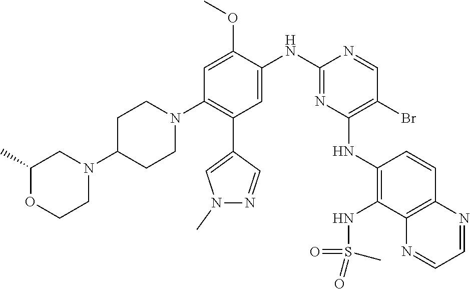 [SMILES: COc1cc(N2CCC(N3CCO[C@H](C)C3)CC2)c(-c2cnn(C)c2)cc1Nc1ncc(Br)c(Nc2ccc3nccnc3c2NS(C)(=O)=O)n1] | (R)-N-(6-((5-bromo-2-((2- methoxy-5-(1-methyl-1H- pyrazol-4-yl)-4-(4-(2- methylmorpholino)piperidin-1- yl)phenyl)amino)pyrimidin-4- yl)amino)quinoxalin-5- yl)methanesulfonamide |
| 84 | 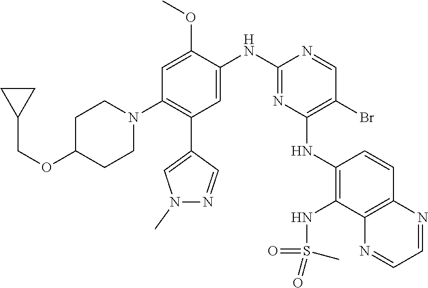 [SMILES: COc1cc(N2CCC(OCC3CC3)CC2)c(-c2cnn(C)c2)cc1Nc1ncc(Br)c(Nc2ccc3nccnc3c2NS(C)(=O)=O)n1] | N-(6-((5-bromo-2-((4-(4- (cyclopropylmethoxy)piperidin- 1-yl)-2-methoxy-5-(1-methyl- 1H-pyrazol-4- yl)phenyl)amino)pyrimidin-4- yl)amino)quinoxalin-5- yl)methanesulfonamide |
| 85 | 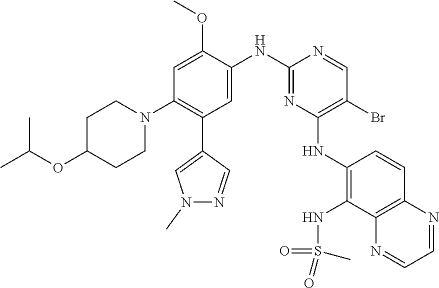 [SMILES: COc1cc(N2CCC(OC(C)C)CC2)c(-c2cnn(C)c2)cc1Nc1ncc(Br)c(Nc2ccc3nccnc3c2NS(C)(=O)=O)n1] | N-(6-((5-bromo-2-((4-(4- isopropoxypiperidin-1-yl)-2- methoxy-5-(1-methyl-1H- pyrazol-4- yl)phenyl)amino)pyrimidin-4- yl)amino)quinoxalin-5- yl)methanesulfonamide |
| 86 | 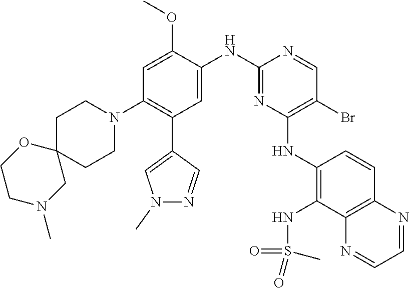 [SMILES: COc1cc(N2CCC3(CC2)CN(C)CCO3)c(-c2cnn(C)c2)cc1Nc1ncc(Br)c(Nc2ccc3nccnc3c2NS(C)(=O)=O)n1] | N-(6-((5-bromo-2-((2-methoxy- 5-(1-methyl-1H-pyrazol-4-yl)-4- (4-methyl-1-oxa-4,9- diazaspiro[5.5]undecan-9- yl)phenyl)amino)pyrimidin-4- yl)amino)quinoxalin-5- yl)methanesulfonamide |
| 87 | 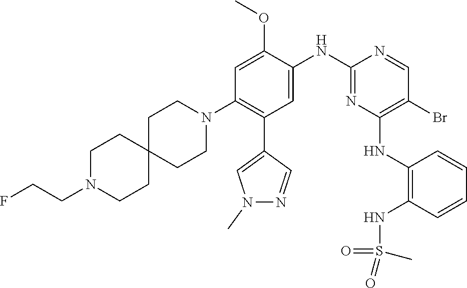 [SMILES: COc1cc(N2CCC3(CCN(CCF)CC3)CC2)c(-c2cnn(C)c2)cc1Nc1ncc(Br)c(Nc2ccccc2NS(C)(=O)=O)n1] | N-(2-((5-bromo-2-((4-(9-(2- fluoroethyl)-3,9- diazaspiro[5.5]undecan-3-yl)-2- methoxy-5-(1-methyl-1H- pyrazol-4- yl)phenyl)amino)pyrimidin-4- yl)amino)phenyl)methanesulfon- amide |
| 88 | 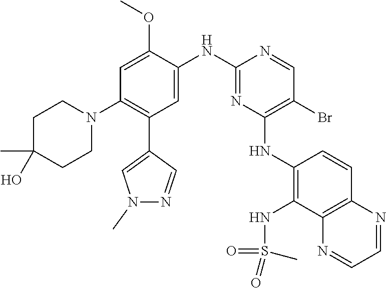 [SMILES: COc1cc(N2CCC(C)(O)CC2)c(-c2cnn(C)c2)cc1Nc1ncc(Br)c(Nc2ccc3nccnc3c2NS(C)(=O)=O)n1] | N-(6-((5-bromo-2-((4-(4- hydroxy-4-methylpiperidin-1-yl)- 2-methoxy-5-(1-methyl-1H- pyrazol-4- yl)phenyl)amino)pyrimidin-4- yl)amino)quinoxalin-5- yl)methanesulfonamide |
| 89 | 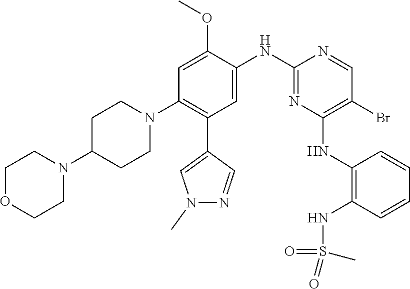 [SMILES: COc1cc(N2CCC(N3CCOCC3)CC2)c(-c2cnn(C)c2)cc1Nc1ncc(Br)c(Nc2ccccc2NS(C)(=O)=O)n1] | N-(2-((5-bromo-2-((2-methoxy- 5-(1-methyl-1H-pyrazol-4-yl)-4- (4-morpholinopiperidin-1- yl)phenyl)amino)pyrimidin-4- yl)amino)phenyl)methanesulfon- amide |
| 90 | 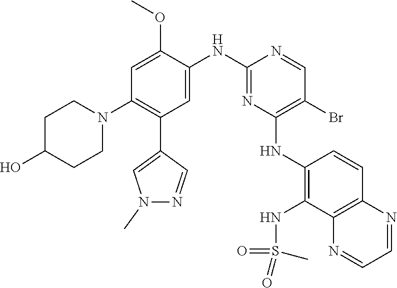 [SMILES: COc1cc(N2CCC(O)CC2)c(-c2cnn(C)c2)cc1Nc1ncc(Br)c(Nc2ccc3nccnc3c2NS(C)(=O)=O)n1] | N-(6-((5-bromo-2-((4-(4- hydroxypiperidin-1-yl)-2- methoxy-5-(1-methyl-1H- pyrazol-4- yl)phenyl)amino)pyrimidin-4- yl)amino)quinoxalin-5- yl)methanesulfonamide |
| 91 | 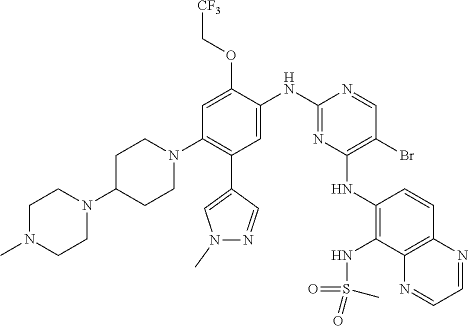 [SMILES: CN1CCN(C2CCN(c3cc(OCC(F)(F)F)c(Nc4ncc(Br)c(Nc5ccc6nccnc6c5NS(C)(=O)=O)n4)cc3-c3cnn(C)c3)CC2)CC1] | N-(6-((5-bromo-2-((5-(1-methyl- 1H-pyrazol-4-yl)-4-(4-(4- methylpiperazin-1-yl)piperidin- 1-yl)-2-(2,2,2- trifluoroethoxy)phenyl)amino)py- rimidin-4-yl)amino)quinoxalin-5- yl)methanesulfonamide |
| 92 | 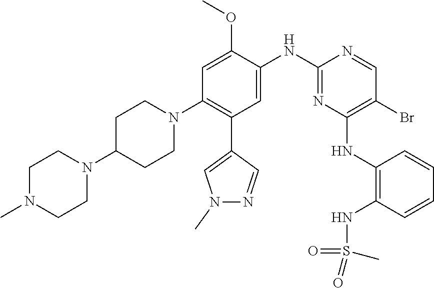 [SMILES: COc1cc(N2CCC(N3CCN(C)CC3)CC2)c(-c2cnn(C)c2)cc1Nc1ncc(Br)c(Nc2ccccc2NS(C)(=O)=O)n1] | N-(2-((5-bromo-2-((2-methoxy- 5-(1-methyl-1H-pyrazol-4-yl)-4- (4-(4-methylpiperazin-1- yl)piperidin-1- yl)phenyl)amino)pyrimidin-4- yl)amino)phenyl)methanesulfon- amide |
| 93 | 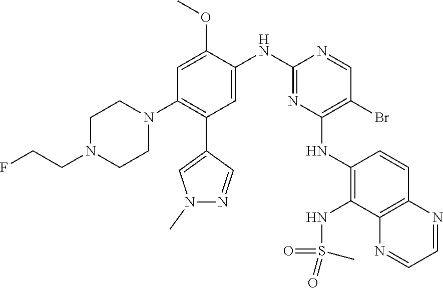 [SMILES: COc1cc(N2CCN(CCF)CC2)c(-c2cnn(C)c2)cc1Nc1ncc(Br)c(Nc2ccc3nccnc3c2NS(C)(=O)=O)n1] | N-(6-((5-bromo-2-((4-(4-(2- fluoroethyl)piperazin-1-yl)-2- methoxy-5-(1-methyl-1H- pyrazol-4- yl)phenyl)amino)pyrimidin-4- yl)amino)quinoxalin-5- yl)methanesulfonamide |
| 94 | 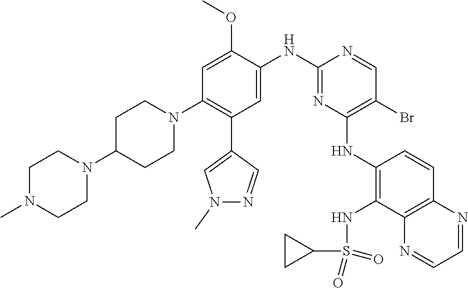 [SMILES: COc1cc(N2CCC(N3CCN(C)CC3)CC2)c(-c2cnn(C)c2)cc1Nc1ncc(Br)c(Nc2ccc3nccnc3c2NS(=O)(=O)C2CC2)n1] | N-(6-((5-bromo-2-((2-methoxy- 5-(1-methyl-1H-pyrazol-4-yl)-4- (4-(4-methylpiperazin-1- yl)piperidin-1- yl)phenyl)amino)pyrimidin-4- yl)amino)quinoxalin-5- yl)cyclopropanesulfonamide |
| 95 | 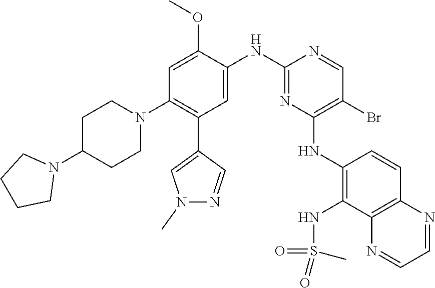 [SMILES: COc1cc(N2CCC(N3CCCC3)CC2)c(-c2cnn(C)c2)cc1Nc1ncc(Br)c(Nc2ccc3nccnc3c2NS(C)(=O)=O)n1] | N-(6-((5-bromo-2-((2-methoxy- 5-(1-methyl-1H-pyrazol-4-yl)-4- (4-(pyrrolidin-1-yl)piperidin-1- yl)phenyl)amino)pyrimidin-4- yl)amino)quinoxalin-5- yl)methanesulfonamide |
| 96 | 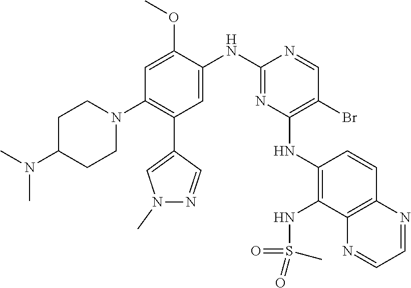 [SMILES: COc1cc(N2CCC(N(C)C)CC2)c(-c2cnn(C)c2)cc1Nc1ncc(Br)c(Nc2ccc3nccnc3c2NS(C)(=O)=O)n1] | N-(6-((5-bromo-2-((4-(4- (dimethylamino)piperidin-1-yl)- 2-methoxy-5-(1-methyl-1H- pyrazol-4- yl)phenyl)amino)pyrimidin-4- yl)amino)quinoxalin-5- yl)methanesulfonamide |
| 97 | 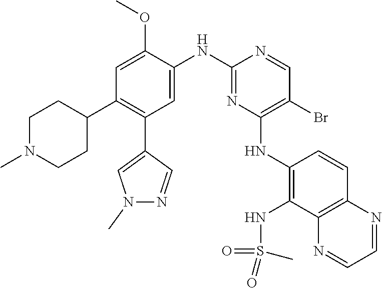 [SMILES: COc1cc(C2CCN(C)CC2)c(-c2cnn(C)c2)cc1Nc1ncc(Br)c(Nc2ccc3nccnc3c2NS(C)(=O)=O)n1] | N-(6-((5-bromo-2-((2-methoxy- 5-(1-methyl-1H-pyrazol-4-yl)-4- (1-methylpiperidin-4- yl)phenyl)amino)pyrimidin-4- yl)amino)quinoxalin-5- yl)methanesulfonamide |
| 98 | 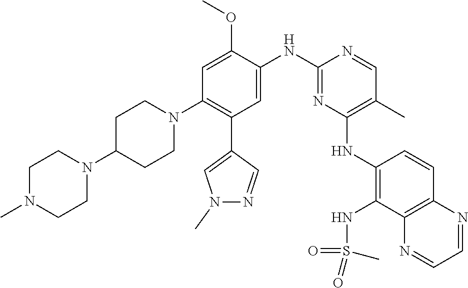 [SMILES: COc1cc(N2CCC(N3CCN(C)CC3)CC2)c(-c2cnn(C)c2)cc1Nc1ncc(C)c(Nc2ccc3nccnc3c2NS(C)(=O)=O)n1] | N-(6-((2-((2-methoxy-5-(1- methyl-1H-pyrazol-4-yl)-4-(4-(4- methylpiperazin-1-yl)piperidin- 1-yl)phenyl)amino)-5- methylpyrimidin-4- yl)amino)quinoxalin-5- yl)methanesulfonamide |
| 99 | 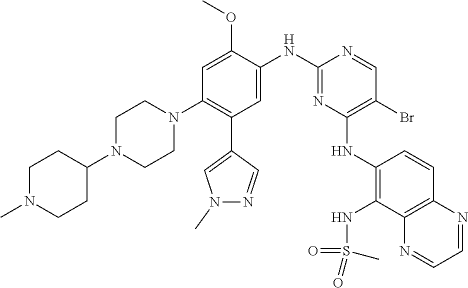 [SMILES: COc1cc(N2CCN(C3CCN(C)CC3)CC2)c(-c2cnn(C)c2)cc1Nc1ncc(Br)c(Nc2ccc3nccnc3c2NS(C)(=O)=O)n1] | N-(6-((5-bromo-2-((2-methoxy- 5-(1-methyl-1H-pyrazol-4-yl)-4- (4-(1-methylpiperidin-4- yl)piperazin-1- yl)phenyl)amino)pyrimidin-4- yl)amino)quinoxalin-5- yl)methanesulfonamide |
| 100 | 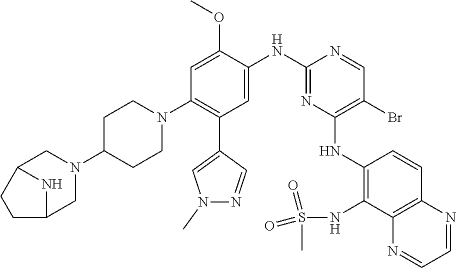 [SMILES: COc1cc(N2CCC(N3CC4CCC(C3)N4)CC2)c(-c2cnn(C)c2)cc1Nc1ncc(Br)c(Nc2ccc3nccnc3c2NS(C)(=O)=O)n1] | N-(6-((2-((4-(4-(3,8- diazabicyclo[3.2.1]octan-3- yl)piperidin-1-yl)-2-methoxy-5- (1-methyl-1H-pyrazol-4- yl)phenyl)amino)-5- bromopyrimidin-4- yl)amino)quinoxalin-5- yl)methanesulfonamide |

### Pharmaceutical Compositions

Pharmaceutical compositions of the present disclosure comprise at least one compound of Formula (I), or tautomer, stereoisomer or a mixture of stereoisomers, or a pharmaceutically acceptable salt, or hydrate, or deuterated derivative thereof formulated together with one or more pharmaceutically acceptable carriers. These formulations include those suitable for oral, rectal, topical, buccal and parenteral (e.g., subcutaneous, intramuscular, intradermal, or intravenous) administration. The most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one compound of the present disclosure as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the present disclosure may be prepared by any of the well-known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of the present disclosure with a liquid or finely divided solid carrier, or both, and then, if necessary, shaping the product. For example, a tablet may be prepared by compressing or molding a powder or granules of at least one compound of the present disclosure, which may be optionally combined with one or more accessory ingredients. Compressed tablets may be prepared by compressing, in a suitable machine, at least one compound of the present disclosure in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the present disclosure is moistened with an inert liquid diluent.

Formulations suitable for buccal (sub-lingual) administration include lozenges comprising at least one compound of the present disclosure in a flavored base, usually sucrose and acacia or tragacanth, and pastilles comprising the at least one compound in an inert base such as gelatin and glycerin or sucrose and acacia.

Formulations of the present disclosure suitable for parenteral administration comprise sterile aqueous preparations of at least one compound of Formula (I), or pharmaceutically acceptable salts thereof, which are approximately isotonic with the blood of the intended recipient. These preparations are administered intravenously, although administration may also be effected by means of subcutaneous, intramuscular, or intradermal injection. Such preparations may conveniently be prepared by admixing at least one compound described herein with water and rendering the resulting solution sterile and isotonic with the blood. Injectable compositions according to the present disclosure may contain from about 0.1 to about 5% w/w of the active compound.

Formulations suitable for rectal administration are presented as unit-dose suppositories. These may be prepared by admixing at least one compound as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one compound of Formula (I), or pharmaceutically acceptable salts thereof) is generally present at a concentration of from about 0.1% to about 15% w/w of the composition, for example, from about 0.5 to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 1 μg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50(4):219-244 (1966) and the following Table for Equivalent Surface Area Dosage Factors).

TABLE 2
Equivalent Surface Area Dosage Factors.

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | 1/2 | 1/4 | 1/6 | 1/12 |
| Rat | 2 | 1 | 1/2 | 1/4 | 1/7 |
| Monkey | 4 | 2 | 1 | 3/5 | 1/3 |
| Dog | 6 | 4 | 3/5 | 1 | 1/2 |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

### Methods of Treatment

The present disclosure provides a compound of Formula (I), or a tautomer, stereoisomer or a mixture of stereoisomers, or a pharmaceutically acceptable salt, or hydrate, or deuterated derivative thereof, to be administered to treat cancer in a subject in need thereof.

In some embodiments, the compound is a mutant-selective EGFR inhibitor.

In some embodiments, the compound inhibits one or more mutations selected from the group consisting of an EGFR mutation, FLT3, and FLT3 mutation. In some embodiments, the EGFR mutation is one or more selected from the group consisting of EGFR del19, EGFR del19/T790M, EGFR del19/T790M/C797S, EGFR L858R, EGFR L858R/T790MS, and EGFR L858R/T790M/C797S.

In some embodiments, the cancer is associated with one or more EGFR mutations.

In some embodiments, the cancer is selected from breast cancer, lung cancer, pancreatic cancer, colon cancer, head and neck cancer, renal cell carcinoma, squamous cell carcinoma, thyroid cancer, gall bladder cancer, thyroid cancer, bile duct cancer, ovarian cancer, endometrial cancer, prostate cancer, or esophageal cancer.

In some embodiments, the cancer is lung cancer. In a further embodiment, the cancer is EGFR-mutated non-small cell lung cancer. In some embodiments, the cancer is pancreatic cancer. In some embodiments, the cancer is colon cancer. In some embodiments, the cancer is breast cancer.

In some embodiments, a compound of Formula (I), or a tautomer, stereoisomer or a mixture of stereoisomers, or a pharmaceutically acceptable salt, or hydrate, or deuterated derivative thereof, is administered as a pharmaceutical composition.

The concentration and route of administration to the patient will vary depending on the cancer to be treated.

In one embodiment, a compound of Formula (I), or a tautomer, stereoisomer or a mixture of stereoisomers, or a pharmaceutically acceptable salt, or hydrate, or deuterated derivative thereof, is administered in combination with an additional agent. In some embodiments, the additional agent is at least one EGFR inhibitor. In some embodiments, the at least one EGFR inhibitor is chosen from afatinib, ASP8273, avitinib, brigatinib, cetuximab, dacomitinib, EA1045, erlotinib, gefitinib, HS-10296, icotinib, lapatinib, necitumumab, nazartinib, neratinib, olmutinib, osimertinib, panitumumab, PF-06747775, EGF816, YH5448, avitinib, rociletinib, vandetanib, and pharmaceutically acceptable salts thereof. In some embodiments, the additional agent is chosen from osimertinib, cetuximab, gefitinib, lapatinib, and erlotinib.

In some embodiments, a compound of Formula (I), or a tautomer, stereoisomer or a mixture of stereoisomers, or a pharmaceutically acceptable salt, or hydrate, or deuterated derivative thereof, or a pharmaceutical composition comprising the compound of Formula (I), and the additional agent are administered concomitantly. In one embodiment, the compound of Formula (I), or the pharmaceutical composition comprising the compound of Formula (I), and the additional agent are administered sequentially.

Also provided herein is a compound of Formula (I), or a pharmaceutically acceptable salt thereof, or a pharmaceutical composition thereof as defined herein for use in therapy.

Also provided herein is a compound of Formula (I), or a pharmaceutically acceptable salt thereof or a pharmaceutical composition thereof as defined herein for use in the treatment of cancer. In some embodiments, the pharmaceutical composition enhances an anticancer effect by the combination administration with an anticancer agent.

Also provided herein is a compound of Formula (I), or a pharmaceutically acceptable salt thereof for use in the inhibition of EGFR.

Also provided herein is a compound of Formula (I), or a pharmaceutically acceptable salt thereof or a pharmaceutical composition thereof as defined herein, for use in the treatment of a disease or disorder associated with one or more EGFR mutations.

Also provided herein is the use of a compound of Formula (I), or a pharmaceutically acceptable salt thereof, as defined herein in the manufacture of a medicament for the treatment of cancer.

Also provided herein is a use of a compound of Formula (I), or a pharmaceutically acceptable salt thereof, as defined herein in the manufacture of a medicament for the inhibition of activity of EGFR.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

The invention also provides for a method of inhibiting EGFR kinase activity in a cell comprising contacting the cell with an effective amount of an EGFR antagonist. In one embodiment, the administered amount is a therapeutically effective amount and the inhibition of EGFR kinase activity further results in the inhibition of the growth of the cell. In a further embodiment, the cell is a cancer cell.

Inhibition of cell proliferation is measured using methods known to those skilled in the art. For example, a convenient assay for measuring cell proliferation is the CellTiter-Glo™ Luminescent Cell Viability Assay, which is commercially available from Promega (Madison, Wis.). That assay determines the number of viable cells in culture based on quantitation of ATP present, which is an indication of metabolically active cells. See Crouch et al (1993) J. Immunol. Meth. 160:81-88, U.S. Pat. No. 6,602,677. The assay may be conducted in 96- or 384-well format, making it amenable to automated high-throughput screening (HTS). See Cree et al (1995) AntiCancer Drugs 6:398-404. The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU). Inhibition of cell proliferation may also be measured using colony formation assays known in the art.

Furthermore, the invention provides for methods of treating a cancer associated with one or more EGFR mutations in a subject suffering therefrom, comprising administering to the subject a therapeutically effective amount of an EGFR antagonist. In one embodiment, the condition is a cell proliferative disease.

Treatment of the cell proliferative disorder by administration of an EGFR antagonist results in an observable and/or measurable reduction in or absence of one or more of the following: reduction in the number of cancer cells or absence of the cancer cells; reduction in the tumor size; inhibition of cancer cell infiltration into peripheral organs including the spread of cancer into soft tissue and bone; inhibition of tumor metastasis; inhibition, to some extent, of tumor growth; and/or relief to some extent, one or more of the symptoms associated with the specific cancer; reduced morbidity and mortality, and improvement in quality of life issues. To the extent the EGFR antagonist may prevent growth and/or kill existing cancer cells, it may be cytostatic and/or cytotoxic. Reduction of these signs or symptoms may also be felt by the patient.

### EXAMPLES

The examples and preparations provided below further illustrate and exemplify the compounds as disclosed herein and methods of preparing such compounds. It is to be understood that the scope of the present disclosure is not limited in any way by the scope of the following examples and preparations.

The chemical entities described herein can be synthesized according to one or more illustrative schemes herein and/or techniques well known in the art. Unless specified to the contrary, the reactions described herein take place at atmospheric pressure, generally within a temperature range from about −10° C. to about 200° C. Further, except as otherwise specified, reaction times and conditions are intended to be approximate, e.g., taking place at about atmospheric pressure within a temperature range of about −10° C. to about 200° C. over a period that can be, for example, about 1 to about 24 hours; reactions left to run overnight in some embodiments can average a period of about 16 hours.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. See, e.g., Carey et al. Advanced Organic Chemistry, 3^{rd} Ed., 1990 New York: Plenum Press; Mundy et al., Name Reaction and Reagents in Organic Synthesis, 2^{nd} Ed., 2005 Hoboken, NJ: J. Wiley & Sons. Specific illustrations of suitable separation and isolation procedures are given by reference to the examples hereinbelow. However, other equivalent separation or isolation procedures can also be used.

In all of the methods, it is well understood that protecting groups for sensitive or reactive groups may be employed where necessary, in accordance with general principles of chemistry. Protecting groups are manipulated according to standard methods of organic synthesis (T. W. Greene and P. G. M. Wuts (1999) Protective Groups in Organic Synthesis, 3^{rd} Ed., John Wiley & Sons). These groups may be removed at a convenient stage of the compound synthesis using methods that are readily apparent to those skilled in the art.

The compounds described herein can be optionally contacted with a pharmaceutically acceptable acid to form the corresponding acid addition salts. Also, the compounds described herein can be optionally contacted with a pharmaceutically acceptable base to form the corresponding basic addition salts.

In some embodiments, disclosed compounds can generally be synthesized by an appropriate combination of generally well-known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Millipore Sigma or can be readily prepared by those skilled in the art using commonly employed synthetic methodology.

The discussion below is offered to illustrate certain of the diverse methods available for use in making the disclosed compounds and is not intended to limit the scope of reactions or reaction sequences that can be used in preparing the compounds provided herein. The skilled artisan will understand that standard atom valencies apply to all compounds disclosed herein in genus or named compound for unless otherwise specified.

The following abbreviations have the definitions set forth below:
ACN acetonitrile
DCM dichloromethane
DIEA diisopropylethylamine
DIPEA N,N-diisopropylethylamine
DMA N,N-dimethylacetamide
DMF dimethylformamide
DMSO dimethyl sulfoxide
EA ethyl acetate
IPA isopropyl alcohol
LC/MS liquid chromatography/mass spectrometry
MTBE methyl tert-butyl ether
NMP N-methyl-2-pyrrolidone
NMR nuclear magnetic resonance
Pd(dppf)Cl₂ [1,1′-bis(diphenylphosphino)ferrocene]dichloro palladium(II)
PE petroleum ether
Prep-HPLC preparative high performance liquid chromatography
Prep-TLC preparative thin layer chromatography
TFA trifluoroacetic acid
THE tetrahydrofuran

### General Synthetic Schemes

Compounds of Formula I can be prepared according to the method described in the following general schemes. However, the synthesis of these compounds is not limited to these representative methods, as they can also be prepared through various other methods by those skilled in the art of synthetic chemistry.

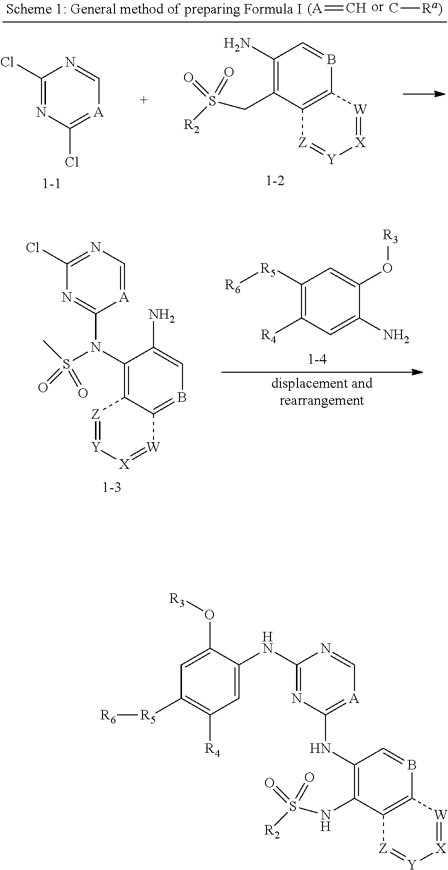

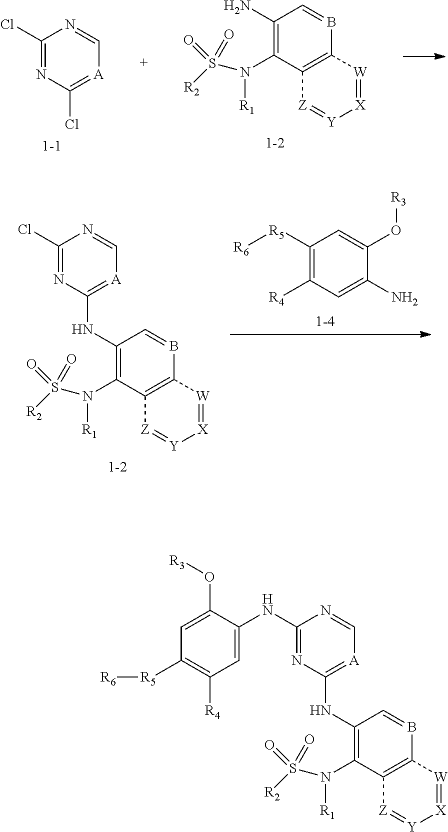

In Scheme 1, substitution of 5-substituted 2,4-dichcloropyrimidines 1-1 with aminosulfonamide 1-2 or 1-2′ under basic condition such as K₂CO₃ in polar solvents such as DMF or DMA can provide intermediate 1-3 or 1-3′. Reaction of intermediate 1-3 with aniline 1-4 under heating conditions can displace the C in 1-3 by the NH₂ group in 1-4 while concomitantly causing an intramolecular rearrangement through a Smiles rearrangement reaction to give the desired product of Formula 1. The reaction of 1-3′ with 1-4 can directly result the desired compound with the structure of Formula I.

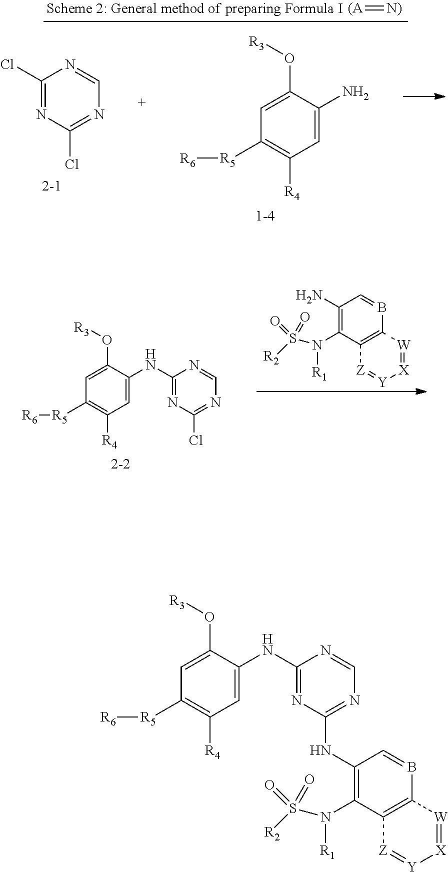

In Scheme 2, the 2,4-dichloro-1,3,5-triazine can react with aniline 1-4 to provide intermediate 2-2, which can react with aminosulfonamide to generate the desired product of Formula I.

For the first step reactions in scheme 1 and 2, they can be carried out in the presence of a base such as DIEA, K₂CO₃, Cs₂CO₃ or NaHCO₃ in solvents such as MeCN, 1,4-dioxane, DMF, DMA, NMP, DMSO, EtOH, IPA, n-BuOH under the condition of conventional or microwave heating.

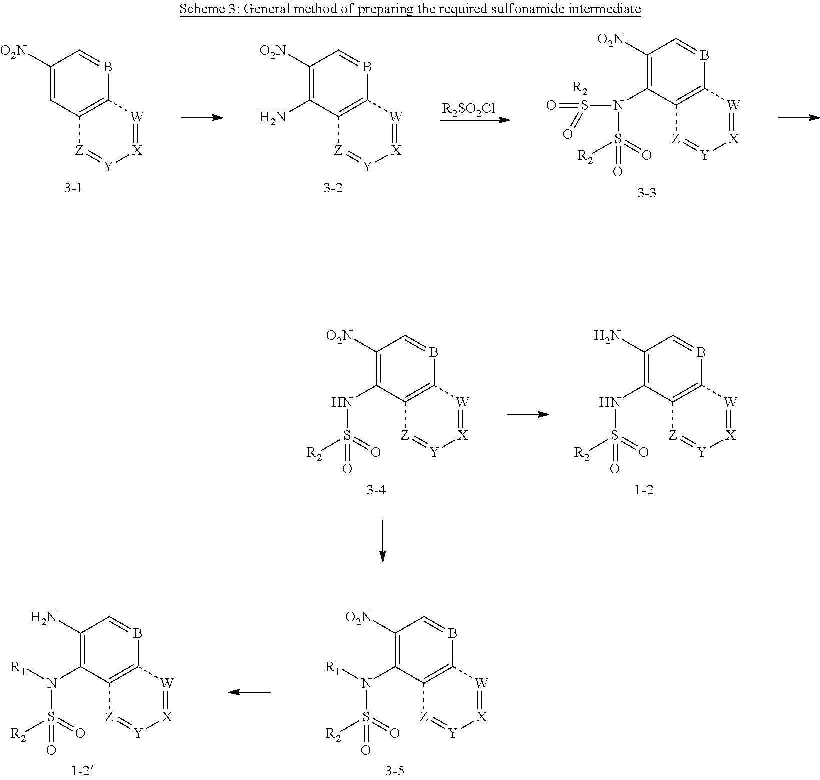

The required hetero bicyclic arylaminosulfoamide 1-2 and 1-2′ can be prepared according to Scheme 3. The nitro substituted bicyclic aryl 3-1 can react with hydroxylamine followed by the elimination to provide 3-2. The amino group in 3-2 can be functionalized by reacting with R₂SO₂Cl to give a bis-sulfonamide 3-3 which can be hydrolyzed to generate a mono-sulfonamide 3-4. Conversion of 3-4 to 1-2 can be accomplished under hydrogenation conditions, such as catalytic hydrogenation. Alkylation of sulfonamide 3-4 can provide 3-5 which can be converted to 1-2′ through the reduction of the nitro group.

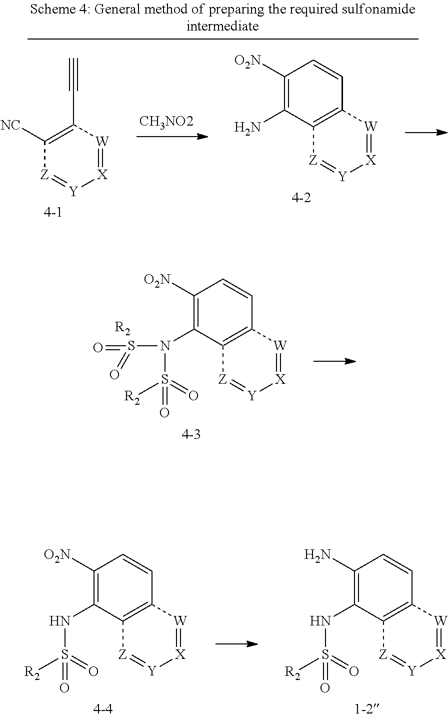

Alternatively, the required intermediate 1-2″ can also be synthesized according to Scheme 4. The 1-cyano-2-ethynyl substituted aromatic 4-1 can react with nitromethane under basic condition in DMSO through an Aza-Hendry reaction to generate the amino-nitro-bicyclic aromatic 4-2, The conversion of 4-2 to 1-2″ can be accomplished as described in Scheme 3.

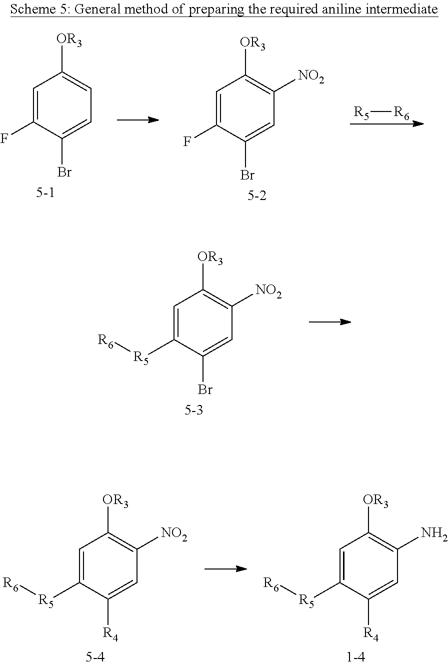

The general method of preparing the required aniline 1-4 is described in Scheme 5. Nitration of 5-1 can generate nitro compound 5-2, which can undergo nucleophilic aromatic substitution to displace F at the para-position by R₅-R₆ to generate 5-3, where R₅ is a cyclic amine. The bromo group in 5-3 can be displaced by R₄ through a palladium catalyzed reaction using alky or alkenyl borate to generate 5-4. Reduction of nitro group in 5-4 under the condition such as hydrogenation will provide the needed aniline 1-4.

### EXAMPLES

### Example 1: Preparation of N-(6-((5-bromo-2-((5-methyl-2-methoxy-4-(4-(4-methylpiperazin-1-yl)piperidin-1-yl)phenyl)amino)pyrimidin-4-yl)amino)quinoxalin-5-yl)methanesulfonamide (Compound 1)

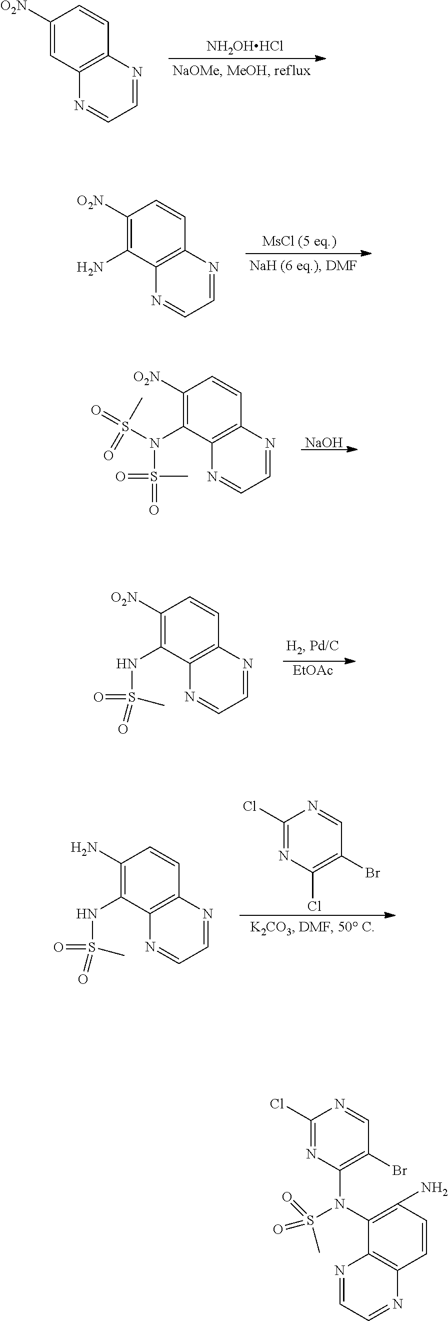

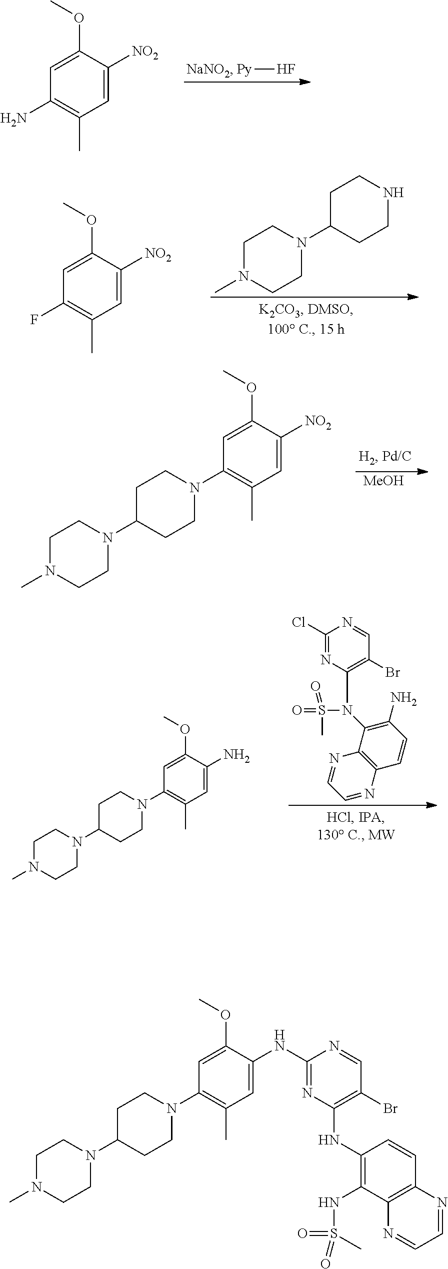

### Step 1: Preparation of 6-nitroquinoxalin-5-amine

To a solution of hydroxylamine hydrochloride (2.98 g, 42.8 mmol) in methanol (150 mL) was added sodium methoxide (5 M, 18.8 mL) at 0° C. The resulting sodium chloride precipitate was allowed to settle before the supernatant solution was added to a well stirred suspension of 6-nitroquinoxaline (5.00 g, 28.6 mmol) in methanol (235 mL) at 80° C. The resulting brown reaction mixture was stirred at 80° C. for 1.5 hr. The reaction mixture was cooled to 0° C. The resulting yellow precipitate was collected by filtration before re-crystallization from a mixture of acetic acid and water (3:1, 150 mL) to afford desired compound (2.8 g, 52% yield) as a yellow solid. ¹H NMR (400 MHz, DMSO-d₆) δ 9.09 (d, J=1.6 Hz, 1H), 8.93 (d, J=1.6 Hz, 1H), 8.50 (br s, 2H), 8.29 (d, J=9.6 Hz, 1H), 7.18 (d, J=9.6 Hz, 1H)

### Step 2 and 3: Preparation of N-(6-nitroquinoxalin-5-yl)methanesulfonamide

To a solution of 6-nitroquinoxalin-5-amine (1.1 g, 5.78 mmol) in dimethylformamide (25 mL) was added sodium hydride (1.39 g, 34.7 mmol, 60% purity) in batches at 60° C. The solution was stirred at 60° C. for 30 min and methanesufonyl chloride (3.31 g, 28.9 mmol) was added at 60° C. The mixture was stirred at 60° C. for another 1 hr to give the di-mesyl intermediate. Sodium hydroxide aqueous solution (3 M, 10 mL) was added into reaction mixture at 20° C., the mixture was stirred at 20° C. for 5 min. The reaction mixture was poured into water (200 mL), and then extracted with ethyl acetate (100 mL×2). The aqueous solution was adjusted with 1 N HCl to pH=4, and then extracted with ethyl acetate (100 mL×2). The combined organic layers were washed with brine (100 mL), dried over anhydrous sodium sulfate, filtered and concentrated under reduced pressure to give a residue N-(6-nitroquinoxalin-5-yl)methanesulfonamide (2.1 g, 45% yield) as a yellow solid. ¹H NMR (400 MHz, DMSO-d₆) δ 10.40 (s, 1H), 9.23-9.09 (m, 2H), 8.30 (d, J=9.2 Hz, 1H), 8.17 (d, J=9.2 Hz, 1H), 3.19 (s, 3H)

### Step 4: Preparation of N-(6-aminoquinoxalin-5-yl)methanesulfonamide

To a solution of N-(6-nitroquinoxalin-5-yl)methanesulfonamide (1.9 g, 7.08 mmol) in ethyl acetate (40 mL) was added Pd/C (400 mg, 10% on carbon) under nitrogen. The mixture was stirred at 20° C. for 1 hr under hydrogen (15 psi). The reaction mixture was filtered and concentrated under reduced pressure to afford desired compound (1.65 g, crude) as a brown solid. ¹H NMR (400 MHz, DMSO-d₆) δ 9.01 (s, 1H), 8.74 (d, J=2.0 Hz, 1H), 8.55 (d, J=2.0 Hz, 1H), 7.77 (d, J=9.2 Hz, 1H), 7.42 (d, J=9.2 Hz, 1H), 6.02 (br s, 2H), 2.99 (s, 3H)

### Step 5: Preparation of N-(6-aminoquinoxalin-5-yl)-N-(5-bromo-2-chloropyrimidin-4-yl)methanesulfonamide

To a solution of N-(6-aminoquinoxalin-5-yl)methanesulfonamide (1.45 g, 6.09 mmol) in dimethylformamide (15 mL) was added potassium carbonate (1.68 g, 12.2 mmol) and 5-bromo-2,4-dichloro-pyrimidine (2.77 g, 12.2 mmol). The mixture was stirred at 50° C. for 12 hr. The reaction mixture was poured into water (20 mL), and then extracted with ethyl acetate (20 mL×2). The combined organic layers were washed with brine (50 mL×3), dried over anhydrous sodium sulfate, filtered and concentrated under reduced pressure to give a residue. The residue was purified by column chromatography with 33%˜50% ethyl acetate in petroleum ether to afford desired compound (790 mg, 30%) as a yellow solid. ¹H NMR (400 MHz, DMSO-d₆) δ 8.62 (s, 1H), 8.49 (d, J=2.0 Hz, 1H), 8.47 (d, J=2.0 Hz, 1H), 7.87 (d, J=9.2 Hz, 1H), 7.41 (d, J=9.2 Hz, 1H), 6.61 (s, 2H, NH₂), 3.65 (s, 3H)

### Step 6: Preparation of 1-fluoro-5-methoxy-2-methyl-4-nitrobenzene

Under an atmosphere of nitrogen, a stirring solution of pyridine (1.7 mL) was treated drop-wise with pyridine hydrofluoride (5.72 g, 40.4 mmol, 70% purity) at −30° C., then 5-methoxy-2-methyl-4-nitro-aniline (1 g, 5.49 mmol) was added and the mixture was treated with sodium nitrite (0.64 g, 9.28 mmol). The reaction was allowed to warm to 25° C. over 45 min and subsequently heated to 60° C. for 2 hr. The reaction mixture was quenched by the addition of ice water (50 mL) and extracted with ethyl acetate (50 mL×2). The combined organic layers were washed with brine (50 mL), dried over anhydrous sodium sulfate, filtered and concentrated under reduced pressure and then purified by column chromatography with 5%˜10% ethyl acetate in petroleum ether to afford the desired compound (0.77 g, 76% yield) as a light yellow solid. ¹H NMR (400 MHz, CDCl₃) δ 7.84 (d, J=8.0 Hz, 1H), 6.76 (d, J=11.2 Hz, 1H), 3.94 (s, 3H), 2.27 (s, 3H). ¹⁹F NMR (377 MHz, CDCl₃) δ −103.840

### Step 7: Preparation of 1-(1-(5-methoxy-2-methyl-4-nitrophenyl)piperidin-4-yl)-4-methylpiperazine

To a solution of 1-fluoro-5-methoxy-2-methyl-4-nitro-benzene (0.57 g, 3.08 mmol) in dimethyl sulfoxide (6 mL) was added potassium carbonate (638.21 mg, 4.62 mmol) and 1-methyl-4-(4-piperidyl)piperazine (564.28 mg, 3.08 mmol). The mixture was stirred at 100° C. for 15 hr. The mixture was partitioned between ethyl acetate (30 mL) and water (30 mL). The organic phase was separated, washed with brine (30 mL), dried over anhydrous sodium sulfate, filtered and concentrated under reduced pressure. The crude product was triturated with methyl tert-butyl ether (10 mL) and filtered. The solid was collected and dried in vacuum to afford desired compound (1.1 g, 75% yield) as a yellow solid. ¹H NMR (400 MHz, CDCl₃) δ 7.82 (s, 1H), 6.54 (s, 1H), 3.94 (s, 3H), 3.35 (d, J=12.4 Hz, 2H), 2.75-2.63 (m, 6H), 2.63-2.44 (m, 4H), 2.43-2.38 (m, 1H), 2.34 (s, 3H), 2.24 (s, 3H), 2.00 (d, J=12.4 Hz, 2H), 1.82-1.62 (m, 2H)

### Step 8: Preparation of 2-methoxy-5-methyl-4-(4-(4-methylpiperazin-1-yl)piperidin-1-yl)aniline

To a solution of 1-[1-(5-methoxy-2-methyl-4-nitro-phenyl)-4-piperidyl]-4-methyl-piperazine (200 mg, 568.25 μmol) in methanol (20 mL) was added Pd/C (40 mg, 10% on carbon). The mixture was stirred at 20° C. for 1 hr under hydrogen (15 Psi). The reaction mixture was filtered and concentrated under reduced pressure to afford desired compound (180 mg, crude) as a yellow solid.

¹H NMR (400 MHz, DMSO-d₆) δ 6.54 (s, 1H), 6.42 (s, 1H), 4.29 (s, 2H), 3.70 (s, 3H), 2.91 (d, J=11.6 Hz, 2H), 2.58-2.51 (m, 4H), 2.44-2.18 (m, 6H), 2.16-2.08 (m, 1H), 2.05 (s, 3H), 1.79 (d, J=11.6 Hz, 2H), 1.61-1.41 (m, 2H)

### Step 9: Preparation of N-(6-((5-bromo-2-((2-methoxy-5-methyl-4-(4-(4-methylpiperazin-1-yl)piperidin-1-yl)phenyl)amino)pyrimidin-4-yl)amino)quinoxalin-5-yl)methanesulfonamide

Dissolve N-(6-aminoquinoxalin-5-yl)-N-(5-bromo-2-chloro-pyrimidin-4-yl)methanesulfonamide (20.0 mg, 46.5 μmol) in HCl/MeOH (4 M, 2.0 mL) and then concentrated under vacuum. The residue and 2-methoxy-5-methyl-4-[4-(4-methylpiperazin-1-yl)-1-piperidyl]aniline (19.1 mg, 60.0 μmol) were combined and taken up into a microwave tube in isopropanol (2 mL). The sealed tube was heated at 130° C. for 2 hr under microwave radiation. Five batches were performed in parallel. The reaction mixtures were combined and concentrated under reduced pressure. The crude product was purified by prep-HPLC [column: Phenomenex Synergi C18 150×25 mm×10 um; mobile phase: A: water (0.1% TFA), B: ACN; gradient: 12%-42% B over 10 min] to afford desired compound (25.4 mg, 11% yield, TFA salt) as a yellow solid. LC/MS: 713.1 [M+H]⁺; ¹HNMR (400 MHz, MeOD-d₄) δ 9.00 (d, J=1.6 Hz, 1H), 8.92 (d, J=1.6 Hz, 1H), 8.54 (br d, J=8.8 Hz, 1H), 8.25 (s, 1H), 8.00 (d, J=9.2 Hz, 1H), 7.43 (s, 1H), 6.81 (s, 1H), 3.85 (s, 3H), 3.27-3.09 (m, 9H), 3.01 (s, 3H), 2.96-2.85 (m, 4H), 2.82 (s, 3H), 2.13-2.09 (m, 2H), 2.02 (s, 3H), 1.89-1.81 (m, 2H)

### Example 2: Preparation of N-(6-((5-bromo-2-((5-ethyl-2-methoxy-4-(4-(4-methylpiperazin-1-yl)piperidin-1-yl)phenyl)amino)pyrimidin-4-yl)amino)quinoxalin-5-yl)methanesulfonamide (Compound 2)

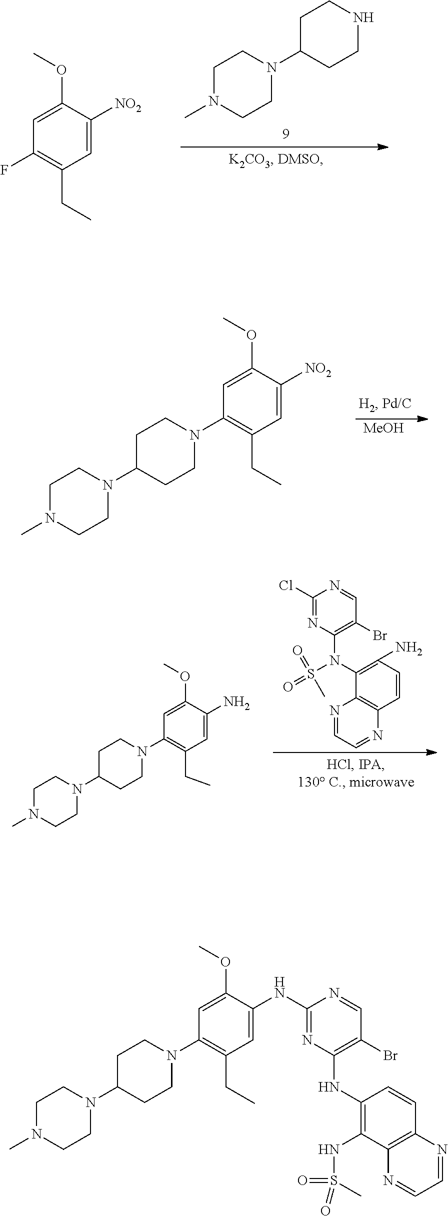

### Step 1: Preparation of 1-(1-(2-ethyl-5-methoxy-4-nitrophenyl)piperidin-4-yl)-4-methylpiperazine

To a solution of 1-ethyl-2-fluoro-4-methoxy-5-nitro-benzene (400 mg, 2.01 mmol) in dimethyl sulfoxide (6 mL) was added potassium carbonate (416 mg, 3.01 mmol) followed by 1-methyl-4-(4-piperidyl)piperazine (480 mg, 2.62 mmol). The solution was stirred at 100° C. for 10 hr. The solution was partitioned between ethyl acetate (50 mL) and water (150 mL). The aqueous layer was extracted with ethyl acetate (30 mL×2). The combined organic layers were washed with brine (30 mL), dried over anhydrous sodium sulfate, filtered and concentrated. The residue was triturated with methyl tert-butyl ether (5 mL) to afford the desired compound (630 mg, 68% yield) as a yellow solid. LC/MS: 363.3 [M+H]⁺.

### Step 2: Preparation of 5-ethyl-2-methoxy-4-(4-(4-methylpiperazin-1-yl)piperidin-1-yl)aniline

To a solution of 1-[1-(2-ethyl-5-methoxy-4-nitro-phenyl)-4-piperidyl]-4-methyl-piperazine (260 mg, 705 μmol) in methanol (30 mL) was added Pd/C (40 mg, 10% on carbon). The mixture was stirred at 20° C. for 1 hr under hydrogen (15 psi). The reaction mixture was filtered and concentrated under reduced pressure to afford the desired compound (230 mg, crude) as a brown solid. ¹H NMR (400 MHz, DMSO-d₆) δ 6.59 (s, 1H), 6.46 (s, 1H), 4.34 (s, 2H), 3.70 (s, 3H), 2.85 (d, J=11.6 Hz, 2H), 2.63-2.55 (m, 4H), 2.48-2.43 (m, 3H), 2.37-2.17 (m, 6H), 2.14 (s, 3H), 1.79 (d, J=11.2 Hz, 2H), 1.54-1.48 (m, 2H), 1.08 (t, J=7.6 Hz, 3H).

### Step 3: Preparation of N-(6-((5-bromo-2-((5-ethyl-2-methoxy-4-(4-(4-methylpiperazin-1-yl)piperidin-1-yl)phenyl)amino)pyrimidin-4-yl)amino)quinoxalin-5-yl)methanesulfonamide

To the mixture of 5-ethyl-2-methoxy-4-[4-(4-methylpiperazin-1-yl)-1-piperidyl]aniline (100 mg, 303 μmol) in isopropanol (10 mL) was added N-(6-aminoquinoxalin-5-yl)-N-(5-bromo-2-chloro-pyrimidin-4-yl)methanesulfonamide (100 mg, 232 μmol) and HCl/dioxane (4 M, 291 μL). The sealed tube was heated at 130° C. for 2 hr under microwave condition. The reaction mixture was concentrated under reduced pressure to give a residue. The crude product was purified by prep-HPLC [column: Phenomenex Luna C18 75×30 mm, 3 μm; mobile phase: A: water (0.1% TFA), B: ACN; gradient: B % 20%-40% over 7 min]. Pure fractions were adjusted with sodium hydrogen carbonate solid to pH=8, and then extracted with a mixed solvent dichloromethane/isopropanol (3:1, 20 mL×2). The combined organic layers were concentrated and then lyophilized to afford N-(6-((5-bromo-2-((5-ethyl-2-methoxy-4-(4-(4-methylpiperazin-1-yl)piperidin-1-yl)phenyl)amino)pyrimidin-4-yl)amino)quinoxalin-5-yl)methanesulfonamide (61.89 mg, 33% yield) as a yellow solid. LC/MS: 725.3 [M+H]⁺; ¹H NMR (400 MHz, CDCl₃) δ 9.12 (s, 1H), 8.90 (d, J=1.6 Hz, 1H), 8.84 (d, J=1.6 Hz, 1H), 8.72 (d, J=9.6 Hz, 1H), 8.26 (s, 1H), 8.05 (d, J=9.6 Hz, 1H), 7.88 (s, 1H), 7.40 (s, 1H), 6.63 (s, 1H), 3.84 (s, 3H), 3.06 (d, J=12.0 Hz, 2H), 2.95 (s, 3H), 2.82-2.58 (m, 10H), 2.44-2.38 (m, 6H), 2.00-1.90 (m, 2H), 1.73-1.67 (m, 2H), 0.78-0.72 (m, 3H); ¹H NMR (400 MHz, DMSO-d₆) δ 9.93 (br s, 1H), 9.09 (s, 1H), 8.94 (s, 1H), 8.87 (s, 1H), 8.73-8.58 (m, 1H), 8.26 (s, 1H), 8.17 (s, 1H), 7.79-7.68 (m, 1H), 7.41 (s, 1H), 6.77 (s, 1H), 3.76 (s, 3H), 3.00 (s, 5H), 2.68 (d, J=4.8 Hz, 3H), 2.53-2.51 (m, 3H), 2.46-2.30 (m, 7H), 2.15 (s, 3H), 1.88-1.73 (m, 2H), 1.62-1.52 (m, 2H), 0.90-0.82 (m, 3H).

### Example 3: Preparation of N-(2-((4-((2-methoxy-5-methyl-4-(4-(4-methylpiperazin-1-yl)piperidin-1-yl)phenyl)amino)-1,3,5-triazin-2-yl)amino)phenyl)methanesulfonamide

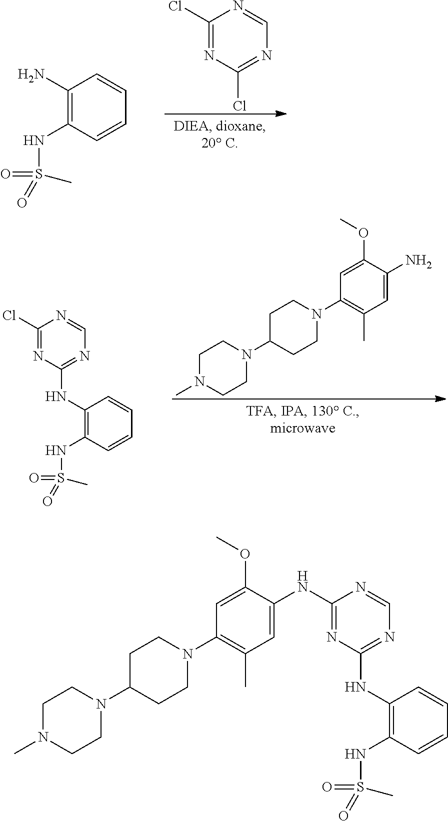 [SMILES: CS(=O)(=O)Nc1ccccc1N ; Clc1ncnc(Cl)n1 ; CS(=O)(=O)Nc1ccccc1Nc1ncnc(Cl)n1 ; COc1cc(N2CCC(N3CCN(C)CC3)CC2)c(C)cc1N ; COc1cc(N2CCC(N3CCN(C)CC3)CC2)c(C)cc1Nc1ncnc(Nc2ccccc2NS(C)(=O)=O)n1]

### Step 1: Preparation of N-(2-((4-chloro-1,3,5-triazin-2-yl)amino)phenyl)methanesulfonamide

To a solution of 2,4-dichloro-1,3,5-triazine (161 mg, 1.07 mmol, 1 eq) and DIEA (158 mg, 1.22 mmol, 1.14 eq) in dioxane (2 mL) was added dropwise a solution of N-(2-aminophenyl)methanesulfonamide (200 mg, 1.07 mmol, 1 eq) in dioxane (2 mL) at 20° C. The resulting mixture was stirred at 20° C. for 5 min. The reaction mixture was partitioned between EtOAc (5 mL) and H₂O (5 mL). The organic phase was separated, washed with brine (5 mL), dried over Na₂SO₄, filtered and concentrated. The residue was purified by silica gel column chromatography (PE/EA=1/1) to give the desired product (180 mg, 42% yield) as yellow oil. LC/MS: 300.3 [M+H]⁺.

### Step 2: Preparation of N-(2-((4-((2-methoxy-5-methyl-4-(4-(4-methylpiperazin-1-yl)piperidin-1-yl)phenyl)amino)-1,3,5-triazin-2-yl)amino)phenyl)methanesulfonamide

N-[2-[(4-chloro-1,3,5-triazin-2-yl)amino]phenyl]methanesulfonamide (20 mg, 66.7 μmol, 1 eq), 2-methoxy-5-methyl-4-[4-(4-methylpiperazin-1-yl)-1-piperidyl]aniline (21.2 mg, 66.7 μmol, 1 eq) and TFA (7.6 mg, 66.7 μmol, 1 eq) were taken up into a microwave tube in IPA (1 mL). The sealed tube was heated at 130° C. for 2 h under microwave irradiation. The reaction mixture was concentrated under reduced pressure to give a residue. The crude product was purified by prep-HPLC [column: Phenomenex Gemini-NX C18 75×30 mm, 3 μm; mobile phase: water (0.05% ammonia hydroxide v/v)-ACN; B %: 15%-45%, 7 min]. The partially purified product was further purified by prep-HPLC [column: Waters Xbridge 150×25 mm, 5 μm; mobile phase: water (10 mM NH₄HCO₃)-ACN; B %: 22%-52%, in 8 min] to give the product (0.71 mg, 1.6% yield, 94.2% purity) as a white solid. LC/MS: 582.5 [M+H]⁺; ¹H NMR (400 MHz, MeOD-d₄) δ 8.21 (s, 1H), 7.86-7.73 (m, 1H), 7.69-7.61 (m, 1H), 7.45 (d, J=9.2 Hz, 1H), 7.32-7.22 (m, 2H), 6.71 (s, 1H), 3.83 (s, 3H), 3.21-3.11 (m, 2H), 2.90 (s, 3H), 2.77-2.41 (m, 11H), 2.32 (s, 3H), 2.22-2.07 (m, 3H), 2.06-1.96 (m, 2H), 1.76-1.65 (m, 2H).

### Example 4: Preparation of N-(6-((5-chloro-2-((2-methoxy-5-methyl-4-(4-(4-methylpiperazin-1-yl)piperidin-1-yl)phenyl)amino)pyrimidin-4-yl)amino)quinoxalin-5-yl)methanesulfonamide (Compound 4)

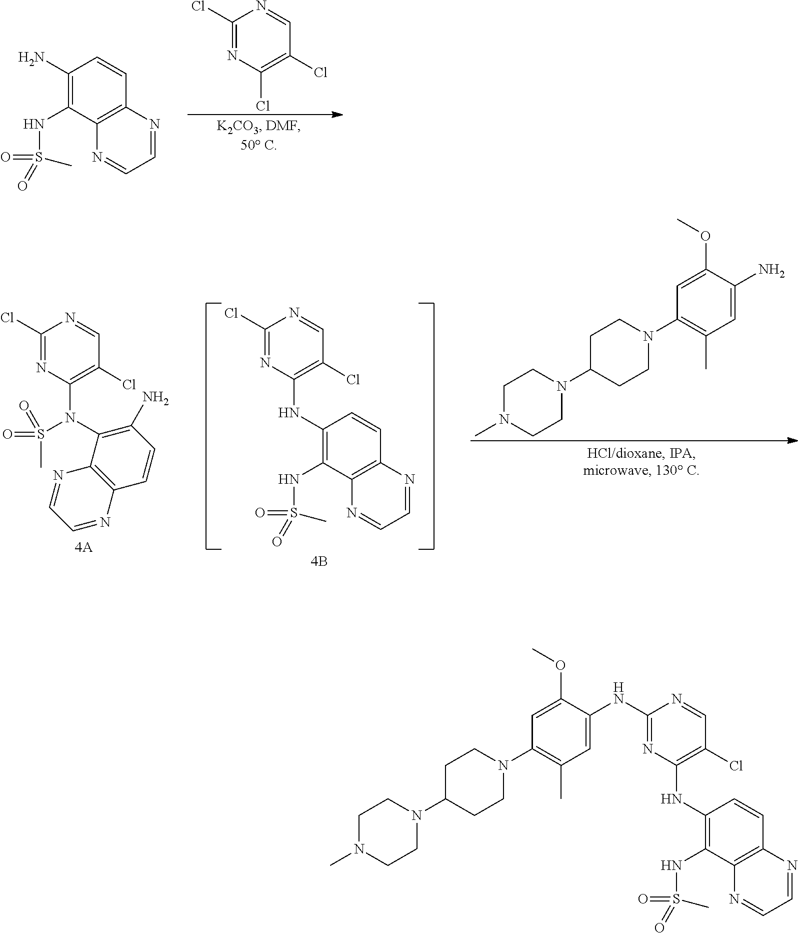

### Step 1: Preparation of N-(6-aminoquinoxalin-5-yl)-N-(2,5-dichloropyrimidin-4-yl)methanesulfonamide and N-(6-((2,5-dichloropyrimidin-4-yl)amino)quinoxalin-5-yl)methanesulfonamide

To a solution of 2,4,5-trichloropyrimidine (154 mg, 839 μmol, 1 eq), N-(6-aminoquinoxalin-5-yl)methanesulfonamide (200 mg, 839 μmol, 1 eq) in DMF (10 mL) was added K₂CO₃ (232 mg, 1.68 mmol, 2 eq). The reaction was stirred at 50° C. for 4 h. The reaction was concentrated under reduced pressure to give a residue. Then H₂O (20 mL) and EtOAc (20 mL) were added and the aqueous phase was extracted with EtOAc (20 mL×2). The combined organic layers were washed with brine (20 mL), dried over anhydrous sodium sulfate, filtered and concentrated under reduced pressure to give a residue. The residue was purified by prep-HPLC [column: Phenomenex Synergi C18 150×25 mm, 10 μm; mobile phase: water (0.1% TFA)-ACN; B %: 34%-64% in 10 min] to afford a mixture of 4A (major) and 4B (minor) with a ratio of 2/1 (60 mg, 19% yield) as a yellow solid. LC/MS: 385.0 [M+H]⁺.

### Step 2: Preparation of N-(6-((5-chloro-2-((2-methoxy-5-methyl-4-(4-(4-methylpiperazin-1-yl)piperidin-1-yl)phenyl)amino)pyrimidin-4-yl)amino)quinoxalin-5-yl)methanesulfonamide

A mixture of N-(6-aminoquinoxalin-5-yl)-N-(2,5-dichloropyrimidin-4-yl)methanesulfonamide (4A) and N-(6-((2,5-dichloropyrimidin-4-yl)amino)quinoxalin-5-yl)methanesulfonamide (4B) (40 mg, 104 μmol, 1 eq), 2-methoxy-5-methyl-4-[4-(4-methylpiperazin-1-yl)-1-piperidyl]aniline (46.3 mg, 120 μmol, 1.16 eq), HCl/dioxane (4 M, 0.13 mL, 5 eq) in IPA (4 mL) were taken up into a microwave tube. The sealed tube was heated at 130° C. for 4 h under microwave irradiation. The reaction mixture was concentrated under reduced pressure to give a residue. The crude product was purified by prep-HPLC (column: Phenomenex Synergi C18 150×25 mm 10 μm; mobile phase: [water (0.1% TFA)-ACN]; B %: 11%-41% in 10 min). The obtained desired fraction was adjusted with NaHCO₃ solid to pH=8 and extracted with CHCl₃/MeOH (20 mL×2, 10/1). The organic layer was concentrated and lyophilized to afford the desired compound (20.39 mg, 28% yield, 95.8% purity) as a yellow solid. LC/MS: 667.2 [M+H]⁺; ¹H NMR (400 MHz, CDCl₃-d) δ 9.23 (s, 1H), 8.89 (d, J=2.0 Hz, 1H), 8.87-8.82 (m, 2H), 8.16 (s, 1H), 8.08 (d, J=9.2 Hz, 1H), 7.88 (s, 1H), 7.39 (s, 1H), 6.58 (s, 1H), 3.84 (s, 3H), 3.12 (d, J=11.6 Hz, 2H), 2.95 (s, 3H), 2.75-2.51 (m, 10H), 2.36 (s, 4H), 2.04 (s, 3H), 1.92 (d, J=11.6 Hz, 2H), 1.73-1.65 (m, 2H)

### Example 5: Preparation of isopropyl 2-((2-methoxy-5-methyl-4-(4-(4-methylpiperazin-1-yl)piperidin-1-yl)phenyl)amino)-4-((5-(methylsulfonamido)quinoxalin-6-yl)amino)pyrimidine-5-carboxylate (Compound 5)

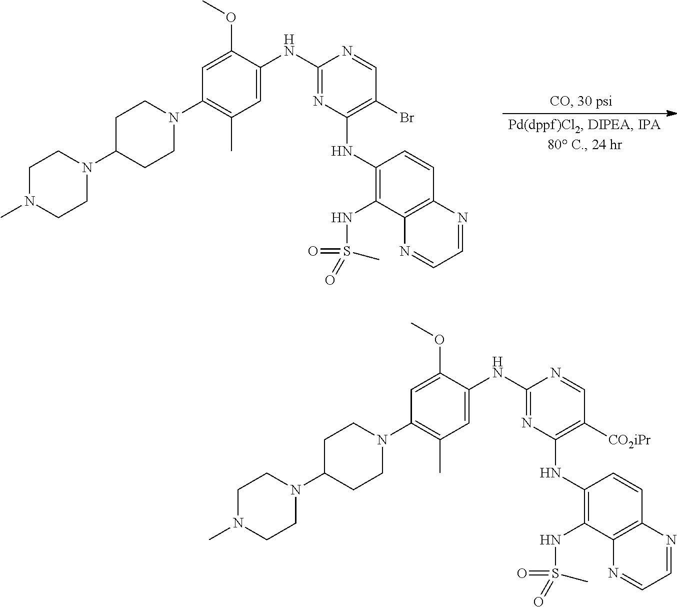

A mixture of N-[6-[[5-bromo-2-[2-methoxy-5-methyl-4-[4-(4-methylpiperazin-1-yl)-1-piperidyl]anilino]pyrimidin-4-yl]amino]quinoxalin-5-yl]methanesulfonamide (Compound 1, 50 mg, 68.5 μmol, 1 eq), DIPEA (17.7 mg, 137 μmol, 2 eq) in IPA (5 mL) was added Pd(dppf)Cl₂ (5 mg, 6.85 μmol, 0.1 eq). The reaction was degassed and purged with CO 3 times. The mixture was stirred at 80° C. for 24 h under CO atmosphere (30 psi). The reaction was filtered and concentrated under reduced pressure to give a residue. The crude product was purified by prep-HPLC (column: Phenomenex Synergi C18 150×25 mm, 10 μm; mobile phase: [water (0.1% TFA)-ACN]; B %: 18%-48% in 10 min). The desired solution was adjusted with NaHCO₃ solid to pH=8 and extracted with CHCl₃/MeOH (20 mL×2, 10/1). The organic layer was concentrated and lyophilized to afford the desired compound (12.94 mg, 26% yield, 98.2% purity) as a yellow solid. LC/MS: 719.4 [M+H]⁺;

¹H NMR (400 MHz, CDCl₃-d) δ 11.27 (s, 1H), 8.96-8.76 (m, 4H), 8.02 (d, J=9.2 Hz, 1H), 7.89 (s, 1H), 7.65 (s, 1H), 6.60 (s, 1H), 5.40-5.32 (m, 1H), 3.85 (s, 3H), 3.14 (d, J=11.6 Hz, 2H), 3.04 (s, 3H), 2.77-2.45 (m, 10H), 2.35-2.29 (m, 4H), 2.01-1.89 (m, 5H), 1.72-1.68 (m, 2H), 1.40 (d, J=6.0 Hz, 6H).

### Example 6: Preparation of N-(6-((5-bromo-2-((2-methoxy-4-(4-(4-methylpiperazin-1-yl)piperidin-1-yl)phenyl)amino)pyrimidin-4-yl)amino)quinoxalin-5-yl)methanesulfonamide (Compound 6)

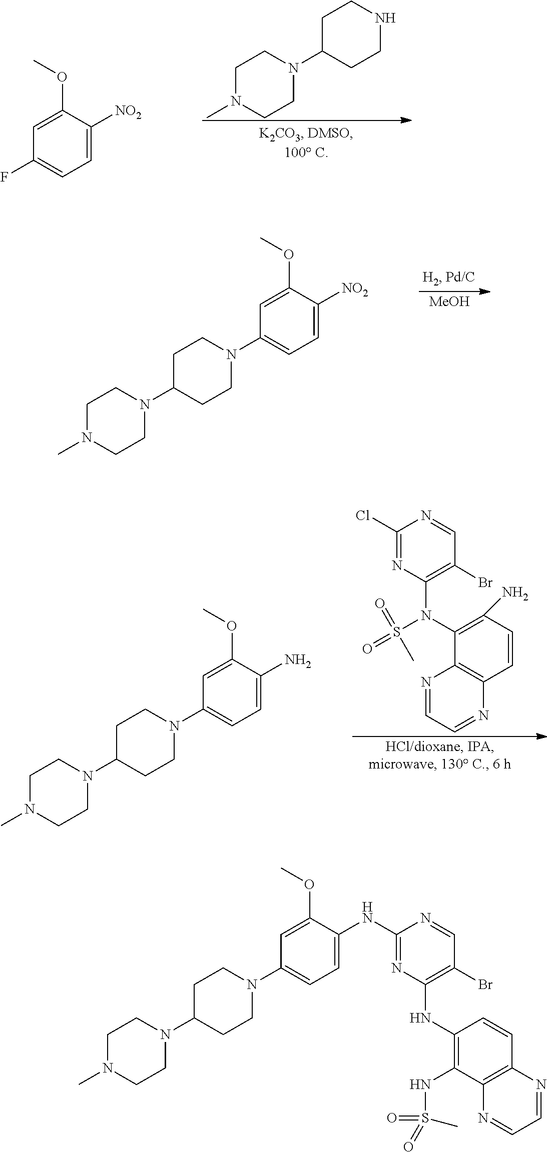

### Step 1: Preparation of 1-(1-(3-methoxy-4-nitrophenyl)piperidin-4-yl)-4-methylpiperazine

To a solution of 4-fluoro-2-methoxy-1-nitro-benzene (0.2 g, 1.17 mmol, 1 eq) in DMSO (5 mL) was added K₂CO₃ (242 mg, 1.75 mmol, 1.5 eq) and 1-methyl-4-(4-piperidyl)piperazine (235 mg, 1.29 mmol, 1.1 eq). The mixture was stirred at 100° C. for 15 hr. The mixture was partitioned between EtOAc (30 mL) and H₂O (30 mL). The organic phase was separated, washed with brine (30 mL), dried over Na₂SO₄, filtered and concentrated under reduced pressure to give a residue. The crude product was triturated with MTBE (10 mL) and filtered. The solid was collected and dried in vacuo to give the product (315 mg, 81% yield) as a yellow solid. LC/MS: 335.3 [M+H]⁺.

### Step 2: Preparation of 2-methoxy-4-(4-(4-methylpiperazin-1-yl)piperidin-1-yl)aniline

To a solution of 1-[1-(3-methoxy-4-nitro-phenyl)-4-piperidyl]-4-methyl-piperazine (315 mg, 942 μmol, 1 eq) in MeOH (20 mL) was added Pd/C (40 mg, 10% purity). The mixture was stirred at 20° C. for 1 hr under H₂ (15 psi). The reaction mixture was filtered and concentrated under reduced pressure to give a product (286 mg, crude) as a brown solid. LC/MS: 305.4 [M+H]⁺.

### Step 3: Preparation of N-(7-((5-bromo-2-((2-methoxy-5-methyl-4-(4-(4-methylpiperazin-1-yl)piperidin-1-yl)phenyl)amino)pyrimidin-4-yl)amino)quinoxalin-6-yl)methanesulfonamide

A mixture of N-(6-aminoquinoxalin-5-yl)-N-(5-bromo-2-chloro-pyrimidin-4-yl)methanesulfonamide (100 mg, 233 μmol, 1 eq), 2-methoxy-4-[4-(4-methylpiperazin-1-yl)-1-piperidyl]aniline (99.2 mg, 326 μmol, 1.4 eq), HCl/dioxane (4 M, 0.25 mL, 4.30 eq) in IPA (10 mL) was degassed and purged with N₂ for 3 times, and then the mixture was stirred at 130° C. for 6 hr under microwave irradiation. The mixture was concentrated. The residue was purified by prep-HPLC (column: Phenomenex Luna C18 75×30 mm, 3 μm; mobile phase: [water (0.1% TFA)-ACN]; B %: 16%-36% in 7 min). The desired solution was adjusted with NaHCO₃solid to pH=8 and extracted with DCM/MeOH (20 mL×2, 10/1). The organic layer was concentrated and lyophilized to give a product (14.27 mg, 8% yield, 97.5% purity) as a yellow solid. LC/MS: 697.1 [M+H]⁺.

¹H NMR (400 MHz, CDCl₃-d) δ 9.12 (s, 1H), 8.89 (s, 1H), 8.84 (t, J=4.4 Hz, 2H), 8.24 (s, 1H), 8.09 (d, J=9.6 Hz, 1H), 7.93 (d, J=8.8 Hz, 1H), 7.30 (s, 1H), 6.53 (s, 1H), 6.40 (d, J=8.8 Hz, 1H), 3.85 (s, 3H), 3.63 (d, J=12.4 Hz, 2H), 2.95 (s, 3H), 2.76-2.60 (m, 7H), 2.59-2.51 (m, 3H), 2.40-2.33 (m, 4H), 1.94 (d, J=12.0 Hz, 2H), 1.70 (d, J=11.2 Hz, 2H).

¹H NMR (400 MHz, DMSO-d₆) δ 9.61 (br s, 1H), 8.83-8.73 (m, 2H), 8.68 (s, 1H), 8.14 (s, 1H), 8.06 (s, 1H), 7.41 (d, J=8.4 Hz, 2H), 6.64 (s, 1H), 6.48 (d, J=8.4 Hz, 1H), 3.78-3.72 (m, 5H), 2.98 (s, 3H), 2.72-2.55 (m, 7H), 2.36-2.26 (m, 4H), 2.14 (s, 3H), 1.86 (d, J=12.0 Hz, 2H), 1.58-1.50 (m, 2H).

### Example 7: Preparation of N-(6-((5-bromo-2-((2-methoxy-5-methyl-4-(4-(1-methylpiperidin-4-yl)piperazin-1-yl)phenyl)amino)pyrimidin-4-yl)amino)quinoxalin-5-yl)methanesulfonamide (Compound 7)

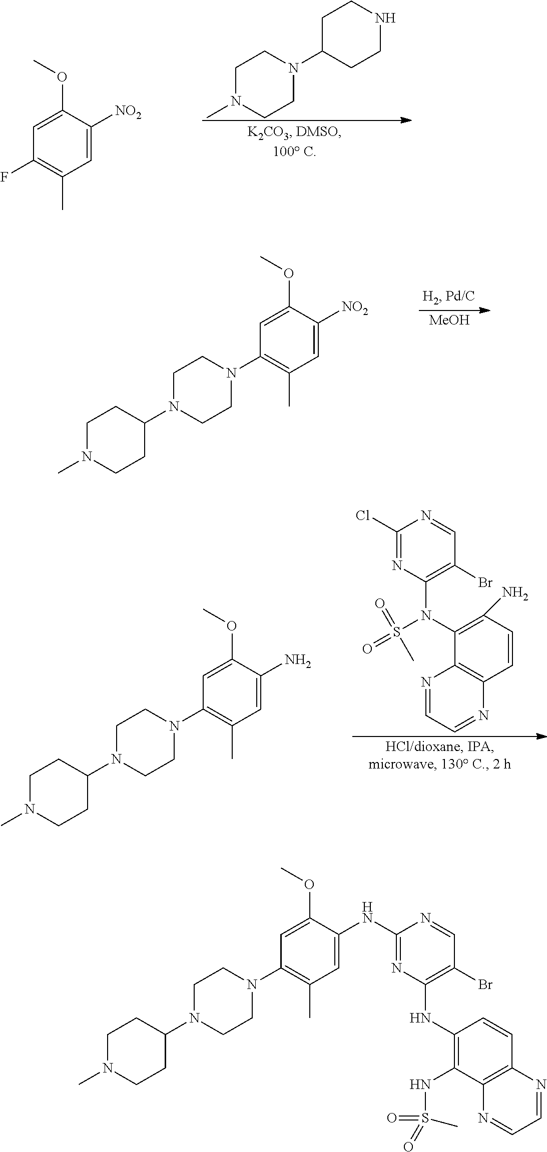

### Step 1: Preparation of 1-(5-methoxy-2-methyl-4-nitrophenyl)-4-(1-methylpiperidin-4-yl)piperazine

To a solution of 1-fluoro-5-methoxy-2-methyl-4-nitro-benzene (150 mg, 810 μmol, 1 eq) and 1-(1-methyl-4-piperidyl)piperazine (178 mg, 972 μmol, 1.2 eq) in DMSO (2 mL) was added K₂CO₃ (224 mg, 1.62 mmol, 2 eq). The mixture was stirred at 100° C. for 16 h. The mixture was diluted with EtOAc (15 mL), washed with brine (30 mL×2). The organic phase was dried over Na₂SO₄, filtered, concentrated to give a residue. To the residue was added MTBE (5 mL) and stirred at 20° C. for 0.5 h. The mixture was filtered, the filter cake was concentrated in vacuo to give product (140 mg, 48% yield) as a red solid. LC/MS: 349.2 [M+H]⁺.

### Step 2: Preparation of 2-methoxy-5-methyl-4-(4-(1-methylpiperidin-4-yl)piperazin-1-yl)aniline

A mixture of 1-(5-methoxy-2-methyl-4-nitro-phenyl)-4-(1-methyl-4-piperidyl)piperazine (140 mg, 386 μmol, 1 eq) in MeOH (5 mL) was degassed and purged with N₂ for 3 times, and then to the mixture was added Pd/C (30 mg, 10% purity). The resulting mixture was degassed and purged with H₂ for 3 times and stirred at 20° C. for 1 hr under H₂ (15 psi). The reaction solution was filtered. The filtrate was concentrated in vacuo to give a desired product (120 mg, crude) as a black oil.

¹H NMR (400 MHz, DMSO-d₆) δ 6.57 (s, 1H), 6.43 (s, 1H), 4.30 (brs, 2H), 3.71 (s, 3H), 3.35-3.30 (m, 4H), 2.80-2.76 (m, 2H), 2.74-2.70 (m, 4H), 2.65-2.55 (m, 3H), 2.13 (s, 3H), 2.06 (s, 3H), 1.76-1.71 (m, 2H), 1.44-1.38 (m, 2H).

### Step 3: Preparation of N-(6-((5-bromo-2-((2-methoxy-5-methyl-4-(4-(1-methylpiperidin-4-yl)piperazin-1-yl)phenyl)amino)pyrimidin-4-yl)amino)quinoxalin-5-yl)methanesulfonamide

To a solution of N-(6-aminoquinoxalin-5-yl)-N-(5-bromo-2-chloro-pyrimidin-4-yl)methanesulfonamide (30.0 mg, 70.0 μmol, 1 eq) and 2-methoxy-5-methyl-4-[4-(1-methyl-4-piperidyl)piperazin-1-yl]aniline (30.0 mg, 94.2 μmol, 1.35 eq) in i-PrOH (3 mL) was added HCl/dioxane (4 M, 75 μL, 4.3 eq). The mixture was stirred at 130° C. for 2 h under microwave irradiation. Three batches were performed in parallel. The reaction solution was concentrated in vacuo to give a residue. The residue was purified by prep-HPLC (column: Phenomenex Luna C18 75×30 mm. 3 μm; mobile phase: [water (0.1% TFA)-ACN]; B %: 16%-36% in 7 min). The desired solution was adjusted with NaHCO₃ solid to pH=8 and extracted with CHCl₃/MeOH (20 mL×2, 10/1), the organic layer was concentrated and lyophilized to give a product (11.27 mg, 96.1% purity) as a green solid. LC/MS: 713.3 [M+H]⁺.

1H NMR (400 MHz, CDCl₃-d) δ 9.14 (s, 1H), 8.89 (d, J=2.0 Hz, 1H), 8.84 (d, J=2.0 Hz, 1H), 8.78 (d, J=9.2 Hz, 1H), 8.25 (s, 1H), 8.06 (d, J=9.2 Hz, 1H), 7.86 (s, 1H), 7.39 (s, 1H), 6.62 (s, 1H), 3.83 (s, 3H), 3.18 (s, 2H), 2.98-2.90 (m, 7H), 2.74 (s, 4H), 2.52 (s, 6H), 2.06-1.94 (m, 7H).

### Example 8: Preparation of N-(6-((5-bromo-2-((4-(4-(dimethylamino)piperidin-1-yl)-2-methoxy-5-methylphenyl)amino)pyrimidin-4-yl)amino)quinoxalin-5-yl)methanesulfonamide (Compound 8)

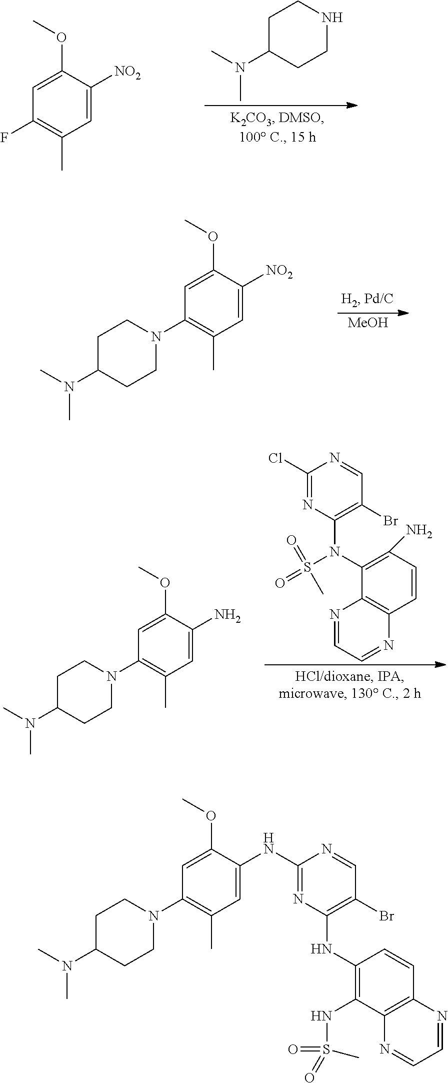

### Step 1: Preparation of 1-(5-methoxy-2-methyl-4-nitrophenyl)-N, N-dimethylpiperidin-4-amine

To a solution of 1-fluoro-5-methoxy-2-methyl-4-nitrobenzene (150 mg, 810 μmol, 1 eq) in DMSO (3 mL) was added K₂CO₃ (168 mg, 1.22 mmol, 1.5 eq), followed by N, N-dimethylpiperidin-4-amine (135 mg, 1.05 mmol, 1.3 eq). The solution was stirred at 100° C. for 12 hr. The solution was partitioned between ethyl acetate (20 mL) and water (50 mL). The aqueous layer was extracted with ethyl acetate (10 mL×5). The combined organic layers were washed with brine (20 mL), dried over sodium sulfate, filtered and concentrated to give a crude product (180 mg) as brown oil, which was used in the next step without further purification. LC/MS: 294.3 [M+H]⁺.

### Step 2: Preparation of 1-(4-amino-5-methoxy-2-methylphenyl)-N, N-dimethylpiperidin-4-amine

To a solution of 1-(5-methoxy-2-methyl-4-nitrophenyl)-N, N-dimethylpiperidin-4-amine (230 mg, 706 μmol, 1 eq) in MeOH (15 mL) under nitrogen atmosphere was added 10% Pd/C (20 mg). The solution was degassed and purged with hydrogen three times. The reaction mixture was stirred under H₂ (15 psi) at 15° C. for 1.5 hr. After filtration through a celite pad, the filtrate was concentrated to give the desired product (180 mg, crude) as a brown oil which was used in the next step. LC/MS: 264.3 [M+H]⁺.

### Step 3: Preparation of N-(6-((5-bromo-2-((4-(4-(dimethylamino)piperidin-1-yl)-2-methoxy-5-methylphenyl)amino)pyrimidin-4-yl)amino)quinoxalin-5-yl)methanesulfonamide

To a solution of N-(6-aminoquinoxalin-5-yl)-N-(5-bromo-2-chloropyrimidin-4-yl)methanesulfonamide (75 mg, 175 μmol, 1 eq) and 1-(4-amino-5-methoxy-2-methylphenyl)-N, N-dimethylpiperidin-4-amine (75 mg, 285 μmol, 1.63 eq) in i-PrOH (7.5 mL) was added HCl/dioxane (4 M, 187 μL, 4.3 eq). The mixture was heated at 130° C. for 2 hr under microwave irradiation. The reaction solution was concentrated in vacuo to give a residue. The residue was purified by prep-HPLC (column: Phenomenex Luna C18 75×30 mm, 3 μm; mobile phase: [water (0.1% TFA)-ACN]; B %: 20%-40% in 7 min). The desired solution was adjusted with NaHCO₃ to pH=8 and extracted with CHCl₃/MeOH (20 mL×2, 10/1). The organic layer was concentrated and further purified by prep-TLC (dichloromethane/methanol=6/1, additional 0.3% NH₃·H₂O) to give a pure product (12.75 mg, 10% yield, 98.3% purity) as a yellow solid. LC/MS: 656.2 [M+H]⁺.

HNMR (400 MHz, CDCl₃-d) δ 9.15 (s, 1H), 8.90 (d, J=2.0 Hz, 1H), 8.85 (d, J=2.0 Hz, 1H), 8.78 (d, J=9.2 Hz, 1H), 8.26 (s, 1H), 8.07 (d, J=9.2 Hz, 1H), 7.87 (s, 1H), 7.39 (s, 1H), 6.58 (s, 1H), 3.85 (s, 3H), 3.20-3.17 (m, 2H), 2.96 (s, 3H), 2.70-2.65 (m, 3H), 2.61 (s, 6H), 2.16-2.12 (m, 2H), 2.01 (s, 3H), 1.86-1.81 (m, 2H).

### Example 9: Preparation of N-(6-((5-bromo-2-((2-methoxy-5-methyl-4-(2-methyl-2,7-diazaspiro[3.5]nonan-7-yl)phenyl)amino)pyrimidin-4-yl)amino)quinoxalin-5-yl)methanesulfonamide (Compound 9)

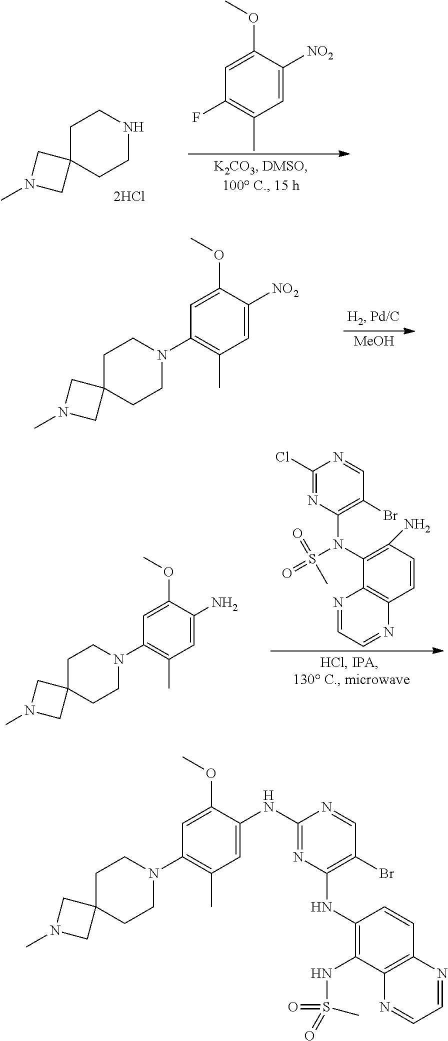

### Step 1: Preparation of 7-(5-methoxy-2-methyl-4-nitrophenyl)-2-methyl-2,7-diazaspiro[3.5]nonane

To a solution of 2-methyl-2,7-diazaspiro[3.5]nonane (250 mg, 1.17 mmol, 1 eq, 2HCl) and 1-fluoro-5-methoxy-2-methyl-4-nitro-benzene (217 mg, 1.17 mmol, 1 eq) in DMSO (5 mL) was added K₂CO₃ (486 mg, 3.52 mmol, 3 eq). The solution was stirred at 100° C. for 12 h. The solution was purified by reverse phase column chromatography (0.1% NH₃·H₂O) to give a desired product (290 mg, 74% yield) as a yellow solid. LC/MS: 306.2 [M+H]⁺.

### Step 2: Preparation of 2-methoxy-5-methyl-4-(2-methyl-2,7-diazaspiro[3.5]nonan-7-yl)aniline

To a solution of 7-(5-methoxy-2-methyl-4-nitro-phenyl)-2-methyl-2,7-diazaspiro[3.5]nonane (270 mg, 813 μmol, 1 eq) in MeOH (5 mL) was added Pd/C (50 mg, 10% purity). The suspension was degassed under vacuum and purged with H₂ several times. The mixture was stirred under H₂ (15 psi) at 20° C. for 1 hr. The suspension was filtered and washed with MeOH (1 mL×3). The combined organic phase was concentrated to give a product (220 mg, crude) as a yellow solid. LC/MS: 276.2 [M+H]⁺.

### Step 3: Preparation of N-(6-((5-bromo-2-((2-methoxy-5-methyl-4-(2-methyl-2,7-diazaspiro[3.5]nonan-7-yl)phenyl)amino)pyrimidin-4-yl)amino)quinoxalin-5-yl)methanesulfonamide

To a solution of N-(6-aminoquinoxalin-5-yl)-N-(5-bromo-2-chloro-pyrimidin-4-yl)methanesulfonamide (100 mg, 232 μmol, 1 eq) in IPA (10 mL) was added HCl/dioxane (4 M, 0.25 mL, 4.30 eq) and 2-methoxy-5-methyl-4-(2-methyl-2,7-diazaspiro[3.5]nonan-7-yl)aniline (89.7 mg, 325 μmol, 1.4 eq). The mixture was stirred at 130° C. for 2 hr under microwave irradiation. The reaction mixture was concentrated under reduced pressure to give a residue. The crude product was purified by prep-HPLC (column: Phenomenex Luna C18 75×30 mm, 3 μm; mobile phase: [water (0.1% TFA)-ACN]; B %: 20%-40% in 7 min) to give a desired product (11.25 mg, 6% yield, 97.6% purity, TFA salt) as a yellow solid. LC/MS: 668.1 [M+H]⁺.

¹H NMR (400 MHz, CDCl₃-d) δ 9.14 (s, 1H), 8.89 (d, J=1.6 Hz, 1H), 8.84 (d, J=1.6 Hz, 1H), 8.79 (d, J=9.6 Hz, 1H), 8.25 (s, 1H), 8.07 (d, J=9.6 Hz, 1H), 7.85 (s, 1H), 7.38 (s, 1H), 6.54 (s, 1H), 3.84 (s, 3H), 3.26 (s, 4H), 2.95 (s, 3H), 2.75-2.70 (m, 4H), 2.49 (s, 3H), 2.00 (s, 3H), 1.92-1.87 (m, 4H).

### Example 10: Preparation of N-(6-((5-bromo-2-((2-methoxy-5-methyl-4-(9-methyl-3,9-diazaspiro[5.5]undecan-3-yl)phenyl)amino)pyrimidin-4-yl)amino)quinoxalin-5-yl)methanesulfonamide (Compound 10)

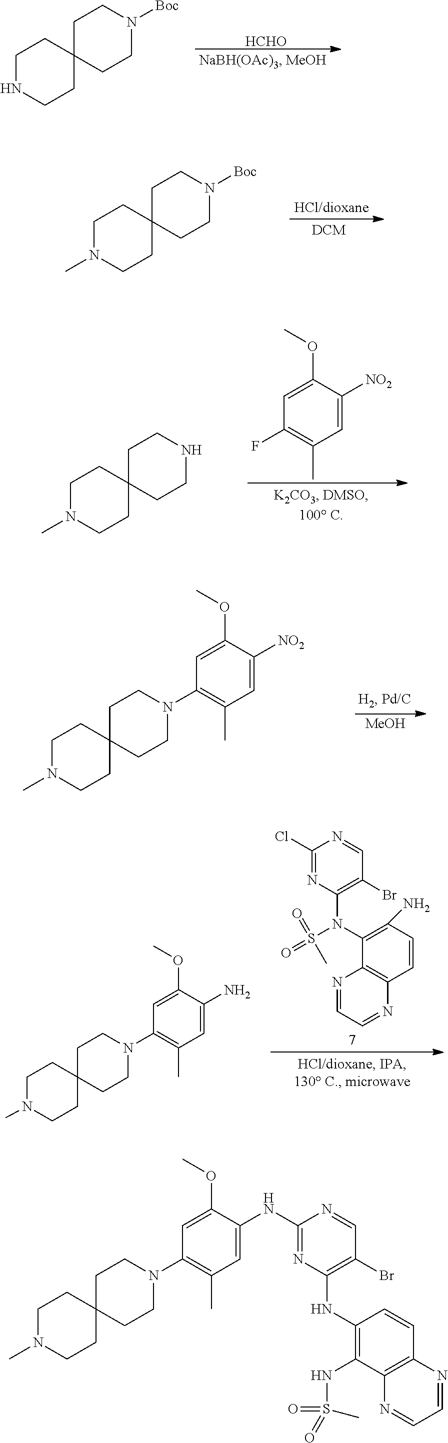

### Step 1: Preparation of tert-butyl 9-methyl-3,9-diazaspiro[5.5]undecane-3-carboxylate

To a solution of tert-butyl 3,9-diazaspiro[5.5]undecane-3-carboxylate (0.7 g, 2.75 mmol, 1 eq) in MeOH (12 mL) was added HCHO (1.12 g, 13.7 mmol, 37% aqueous, 5 eq) at 0° C. The mixture was warmed to 20° C. and stirred for 0.5 hr, then NaBH(OAc)₃ (1.75 g, 8.26 mmol, 3 eq) was added into the mixture, and the mixture was stirred for another 12 hr. The reaction mixture was concentrated. The residue was diluted with H₂O (10 mL) and extracted with DCM (10 mL×2). The combined organic layers were washed with brine (10 mL), dried over Na₂SO₄, filtered and concentrated under reduced pressure to give a product (0.7 g, 95% yield) as a colorless oil.

¹H NMR (400 MHz, CDCl₃-d) δ 3.43-3.34 (m, 4H), 3.31-2.79 (m, 4H), 2.71 (s, 3H), 1.88 (s, 4H), 1.50 (s, 4H), 1.45 (s, 9H).

### Step 2: Preparation of 3-methyl-3,9-diazaspiro[5.5]undecane

To a solution of tert-butyl 9-methyl-3,9-diazaspiro[5.5]undecane-3-carboxylate (0.7 g, 2.61 mmol, 1 eq) in DCM (10 mL) was added HCl/dioxane (4 M, 5 mL, 7.67 eq). The mixture was stirred at 20° C. for 1 hr. The reaction mixture was concentrated under reduced pressure to give a product (620 mg, crude, 2HCl salt) as a yellow oil, which was used directly in next step.

### Step 3: Preparation of 3-(5-methoxy-2-methyl-4-nitrophenyl)-9-methyl-3,9-diazaspiro[5.5]undecane

To a solution of 3-methyl-3,9-diazaspiro[5.5]undecane (0.62 g, 2.57 mmol, 1 eq, 2HCl) in DMSO (10 mL) was added K₂CO₃ (888 mg, 6.43 mmol, 2.5 eq) and 1-fluoro-5-methoxy-2-methyl-4-nitro-benzene (618 mg, 3.34 mmol, 1.3 eq). The mixture was stirred at 100° C. for 16 hr. The reaction mixture was poured into water (20 mL), and then extracted with EtOAc (20 mL×2). The combined organic layers were washed with brine (50 mL), dried over Na₂SO₄, filtered and concentrated under reduced pressure to give a residue. The residue was purified by column chromatography (SiO₂, dichloromethane/methanol=10/1) to give a product (250 mg, 22% yield) as a yellow solid.

¹H NMR (400 MHz, CDCl₃-d) δ 7.83 (d, J=4.0 Hz, 1H), 6.56 (d, J=4.0 Hz, 1H), 3.95 (s, 3H), 2.97 (d, J=4.8 Hz, 4H), 2.63 (d, J=4.4 Hz, 4H), 2.46 (s, 3H), 2.24 (s, 3H), 1.80-1.66 (m, 8H).

### Step 4: Preparation of 2-methoxy-5-methyl-4-(9-methyl-3,9-diazaspiro[5.5]undecan-3-yl)aniline

To a solution of 3-(5-methoxy-2-methyl-4-nitro-phenyl)-9-methyl-3,9-diazaspiro[5.5]undecane (250 mg, 750 μmol, 1 eq) in MeOH (20 mL) was added Pd/C (50 mg, 10% purity) under N₂. The mixture was stirred at 20° C. for 1 hr under H₂ (15 psi). The reaction mixture was filtered and concentrated under reduced pressure to give a product (210 mg, 92% yield) as a brown solid.

¹H NMR (400 MHz, DMSO-d₆) δ 6.61 (s, 1H), 6.42 (s, 1H), 4.28 (brs, 2H), 3.71 (s, 3H), 2.70-2.60 (m, 4H), 2.45-2.35 (m, 4H), 2.25 (s, 3H), 2.05 (s, 3H), 1.55-1.50 (m, 8H).

### Step 5: Preparation of N-(6-((5-bromo-2-((2-methoxy-5-methyl-4-(9-methyl-3,9-diazaspiro[5.5]undecan-3-yl)phenyl)amino)pyrimidin-4-yl)amino)quinoxalin-5-yl)methanesulfonamide

To a solution of N-(6-aminoquinoxalin-5-yl)-N-(5-bromo-2-chloro-pyrimidin-4-yl)methanesulfonamide (100 mg, 232 μmol, 1 eq) in IPA (10 mL) was added HCl/dioxane (4 M, 0.25 mL, 4.30 eq) and 2-methoxy-5-methyl-4-(9-methyl-3,9-diazaspiro[5.5]undecan-3-yl)aniline (98.8 mg, 325 μmol, 1.4 eq). The mixture was heated at 130° C. for 2 hr under microwave irradiation. The reaction mixture was concentrated under reduced pressure to give a residue. The crude product was purified by prep-HPLC (column: Phenomenex Luna C18 75×30 mm, 3 μm; mobile phase: [water (0.1% TFA)-ACN]; B %: 20%-40% in 7 min). The obtained mixture was concentrated under reduced pressure to give a residue and adjusted with a solution of NaHCO₃ (aq.) to pH=8, and then extracted with CHCl₃/MeOH (40 mL×3, 10/1). The combined organic layers were concentrated under reduced pressure and lyophilized to give a product (60.60 mg, 36% yield, 97.7% purity) as a yellow solid. LC/MS: 696.2 [M+H]⁺.

¹H NMR (400 MHz, CDCl₃-d) δ 9.14 (s, 1H), 8.89 (d, J=1.6 Hz, 1H), 8.84 (d, J=1.6 Hz, 1H), 8.81 (d, J=9.2 Hz, 1H), 8.26 (s, 1H), 8.07 (d, J=9.2 Hz, 1H), 7.86 (s, 1H), 7.38 (s, 1H), 6.60 (s, 1H), 3.85 (s, 3H), 3.00-2.90 (s, 5H), 2.81 (s, 5H), 2.70-2.60 (m, 4H), 2.02 (s, 3H), 1.96-1.88 (m, 4H), 1.75-1.66 (m, 4H).

¹H NMR (400 MHz, DMSO-d₆) δ 9.82 (brs, 1H), 8.98 (d, J=1.6 Hz, 1H), 8.93-8.83 (m, 2H), 8.68 (d, J=8.4 Hz, 1H), 8.29 (d, J=16.4 Hz, 1H), 8.19 (s, 1H), 7.86 (d, J=9.6 Hz, 1H), 7.34 (s, 1H), 6.78 (s, 1H), 3.76 (s, 3H), 3.01 (s, 3H), 2.82-2.74 (m, 4H), 2.44 (s, 4H), 2.26 (s, 3H), 2.01 (s, 3H), 1.58-1.50 (m, 8H).

### Example 11: Preparation of N-(6-((5-bromo-2-((2-methoxy-5-methyl-4-(4-(pyrrolidin-1-yl)piperidin-1-yl)phenyl)amino)pyrimidin-4-yl)amino)quinoxalin-5-yl)methanesulfonamide (Compound 11)

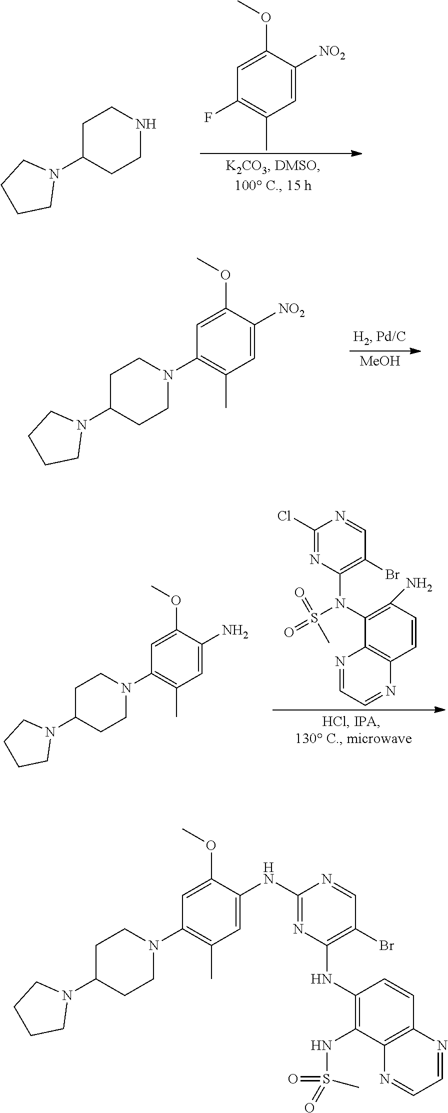

### Step 1: Preparation of 1-(5-methoxy-2-methyl-4-nitrophenyl)-4-(pyrrolidin-1-yl)piperidine

To a solution of 4-pyrrolidin-1-ylpiperidine (250 mg, 1.62 mmol, 1 eq) and 1-fluoro-5-methoxy-2-methyl-4-nitro-benzene (300 mg, 1.62 mmol, 1 eq) in DMSO (5 mL) was added K₂CO₃ (336 mg, 2.43 mmol, 1.5 eq). The solution was stirred at 100° C. for 12 hr. The solution was purified by reverse phase column chromatography (0.1% NH₃·H₂O) to give a product (440 mg, 75% yield) as a yellow solid. LC/MS: 320.2 [M+H]⁺.

### Step 2: Preparation of 2-methoxy-5-methyl-4-(4-(pyrrolidin-1-yl)piperidin-1-yl)aniline

To a solution of 1-(5-methoxy-2-methyl-4-nitro-phenyl)-4-pyrrolidin-1-yl-piperidine (440 mg, 1.21 mmol, 1 eq) in MeOH (10 mL) was added Pd/C (50 mg, 10% purity). The suspension was degassed under vacuum and purged with H₂ three times. The mixture was stirred under H₂ (15 psi) at 20° C. for 1 hr. The suspension was filtered and washed with MeOH (1 mL×3). The filtrate was concentrated to give a product (330 mg, crude) as a yellow solid. LC/MS: 290.2 [M+H]⁺.

### Step 3: Preparation of N-(6-((5-bromo-2-((2-methoxy-5-methyl-4-(4-(pyrrolidin-1-yl)piperidin-1-yl)phenyl)amino)pyrimidin-4-yl)amino)quinoxalin-5-yl)methanesulfonamide

To a solution of N-(6-aminoquinoxalin-5-yl)-N-(5-bromo-2-chloro-pyrimidin-4-yl)methanesulfonamide (100 mg, 232 μmol, 1 eq) in IPA (10 mL) was added HCl/dioxane (4 M, 0.25 mL, 4.30 eq) and 2-methoxy-5-methyl-4-(4-pyrrolidin-1-yl-1-piperidyl)aniline (94.3 mg, 326 μmol, 1.4 eq). The mixture was stirred at 130° C. for 2 hr under microwave irradiation. The reaction mixture was concentrated under reduced pressure to give a residue. The crude product was purified by prep-HPLC (column: Phenomenex Luna C18 75×30 mm, 3 μm; mobile phase: [water (0.1% TFA)-ACN]; B %: 22%-42% in 7 min). The obtained solution was concentrated under reduced pressure to give a residue and adjusted with a solution of NaHCO₃ (aq) to pH=8, and then extracted with CHCl₃/MeOH (40 mL×3, 10/1). The combined organic layers were concentrated. The resulting residue was further purified by prep-TLC (dichloromethane:methanol=10:1) to give a desired product (6.93 mg, 4% yield, 98.2% purity) as a yellow solid. LC/MS: 682.2 [M+H]⁺; ¹H NMR (400 MHz, CDCl₃-d) δ 9.13 (s, 1H), 8.91 (d, J=2.0 Hz, 1H), 8.84 (d, J=2.0 Hz, 1H), 8.79 (d, J=9.2 Hz, 1H), 8.25 (s, 1H), 8.08 (d, J=9.2 Hz, 1H), 7.85 (s, 1H), 7.38 (s, 1H), 6.60 (s, 1H), 3.85 (s, 3H), 3.14-3.09 (m, 2H), 2.95 (s, 3H), 2.85-2.56 (m, 7H), 2.05-1.98 (m, 5H), 1.85-1.76 (m, 6H).

### Example 12: Preparation of N-(6-((5-bromo-2-((2-methoxy-5-methyl-4-(4-(4-methylpiperazin-1-yl)piperidin-1-yl)phenyl)amino)pyrimidin-4-yl)amino)quinoxalin-5-yl)-N-methylmethanesulfonamide (Compound 12)

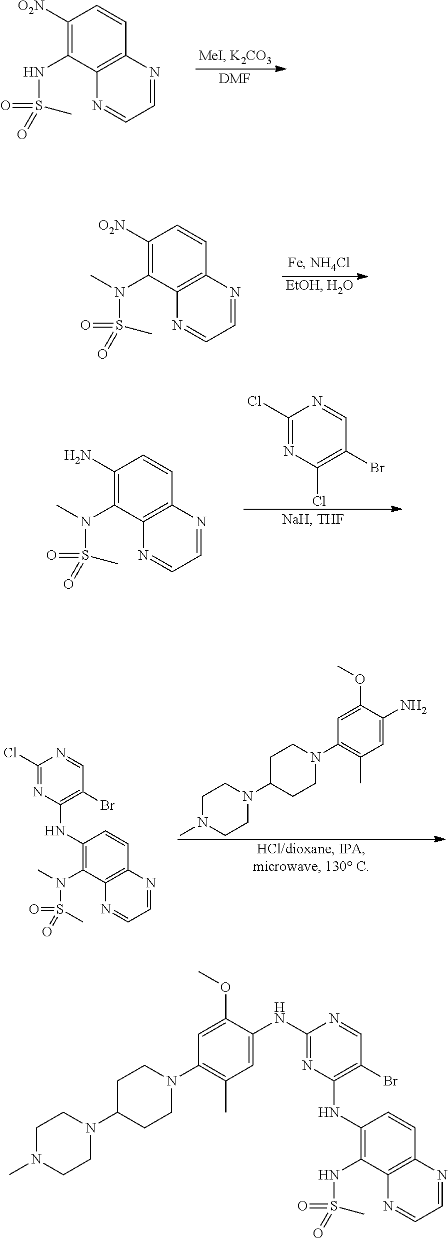

### Step 1: Preparation of N-methyl-N-(6-nitroquinoxalin-5-yl)methanesulfonamide

To a solution of N-(6-nitroquinoxalin-5-yl)methanesulfonamide (0.50 g, 1.86 mmol, 1 eq) in DMF (8 mL) was added Mel (793 mg, 5.59 mmol, 3 eq) and K₂CO₃ (1.03 g, 7.46 mmol, 4 eq). The mixture was stirred at 20° C. for 2 hr. The reaction mixture was poured into water (20 mL), and then extracted with EtOAc (20 mL×2). The combined organic layers were washed with brine (50 mL), dried over Na₂SO₄, filtered and concentrated under reduced pressure to give a product (0.50 g, 95% yield) as a yellow solid.

¹H NMR (400 MHz, CDCl₃-d) δ 9.08-9.03 (m, 2H), 8.30 (d, J=8.8 Hz, 1H), 8.17 (d, J=8.8 Hz, 1H), 3.54 (s, 3H), 3.13 (s, 3H).

### Step 2: Preparation of N-(6-aminoquinoxalin-5-yl)-N-methylmethanesulfonamide

To a solution of N-methyl-N-(6-nitroquinoxalin-5-yl)methanesulfonamide (0.50 g, 1.77 mmol, 1 eq) in EtOH (20 mL) and H₂O (2 mL) was added Fe (495 mg, 8.86 mmol, 5 eq) and NH₄Cl (474 mg, 8.86 mmol, 5 eq). The mixture was stirred at 70° C. for 1 hr. The reaction mixture was filtered and concentrated under reduced pressure to give a residue. The residue was poured into water (20 mL), and then extracted with EtOAc (20 mL×2). The combined organic layers were washed with brine (50 mL), dried over Na₂SO₄, filtered and concentrated under reduced pressure to give a product (0.30 g, 67% yield) as a yellow solid. ¹H NMR (400 MHz, DMSO-d₆) δ 8.72 (s, 1H), 8.55 (s, 1H), 7.80 (d, J=8.8 Hz, 1H), 7.42 (d, J=8.8 Hz, 1H), 6.14 (brs, 2H), 3.23 (s, 3H), 3.18 (s, 3H).

### Step 3: Preparation of N-(6-((5-bromo-2-chloropyrimidin-4-yl)amino)quinoxalin-5-yl)-N-methylmethanesulfonamide

To a solution of N-(6-aminoquinoxalin-5-yl)-N-methyl-methanesulfonamide (100 mg, 396 μmol, 1 eq) in THE (5 mL) was added NaH (20.6 mg, 515 μmol, 60%, 1.3 eq) at 0° C. under N₂. The solution was stirred at 0° C. for 1 hr. Then 5-bromo-2,4-dichloro-pyrimidine (90.3 mg, 396 μmol, 1 eq) was added into the solution and the mixture was stirred at 20° C. for 2 hr. This reaction was repeated with 120 mg of N-(6-aminoquinoxalin-5-yl)-N-methyl-methanesulfonamide. The crude reaction mixtures were combined and poured into solution of NH₄Cl (aq., 20 mL), and extracted with EtOAc (20 mL×2). The combined organic layers were washed with brine (50 mL), dried over Na₂SO₄, filtered and concentrated under reduced pressure to give a product (0.2 g, 95% yield) as a yellow solid. ¹H NMR (400 MHz, DMSO-d₆) b 9.11 (s, 1H), 9.04-8.98 (m, 2H), 8.65 (s, 1H), 8.55 (d, J=9.2 Hz, 1H), 8.25 (d, J=9.2 Hz, 1H), 3.40 (s, 3H), 3.18 (s, 3H).

### Step 4: Preparation of N-(6-((5-bromo-2-((2-methoxy-5-methyl-4-(4-(4-methylpiperazin-1-yl)piperidin-1-yl)phenyl)amino)pyrimidin-4-yl)amino)quinoxalin-5-yl)-N-methylmethanesulfonamide

To a solution of N-[6-[(5-bromo-2-chloro-pyrimidin-4-yl)amino]quinoxalin-5-yl]-N-methyl-methanesulfonamide (100 mg, 225 μmol, 1 eq) in IPA (10 mL) was added HCl/dioxane (4 M, 0.25 mL, 4.44 eq) and 2-methoxy-5-methyl-4-[4-(4-methylpiperazin-1-yl)-1-piperidyl]aniline (100 mg, 315 μmol, 1.4 eq). The mixture was stirred at 130° C. for 2 hr under microwave irradiation. The reaction mixture was concentrated under reduced pressure to give a residue. The crude product was purified by prep-HPLC (column: Phenomenex Luna C18 75×30 mm, 3 μm; mobile phase: [water (0.1% TFA)-ACN]; B %: 22%-42% in 7 min). The desired fractions were concentrated. The residue was adjusted with a solution of NaHCO₃ to pH=8, and then extracted with CHCl₃/MeOH (40 mL×3, 10/1). The combined organic layers were concentrated. The residue was further purified by prep-TLC (dichloromethane:methanol=7:1) to give the title compound (20.53 mg, 12% yield, 96.6% purity) as a yellow solid. LC/MS: 725.3 [M+H]⁺; ¹H NMR (400 MHz, CDCl₃) δ 9.11 (d, J=9.2 Hz, 1H), 8.83 (s, 3H), 8.27 (s, 1H), 8.11 (d, J=9.2 Hz, 1H), 7.96 (s, 1H), 7.37 (s, 1H), 6.63 (s, 1H), 3.87 (s, 3H), 3.51 (s, 3H), 3.22-3.14 (m, 5H), 2.87-2.49 (m, 10H), 2.37 (s, 4H), 2.17 (s, 3H), 2.00-1.94 (m, 2H), 1.78-1.70 (m, 2H).

### Example 15: Preparation of N-(3-{[5-bromo-2-({5-ethyl-2-methoxy-4-[4-(4-methylpiperazin-1-yl)piperidin-1-yl]phenyl}amino)pyrimidin-4-yl]amino}quinolin-4-yl)methanesulfonamide (Compound 15)

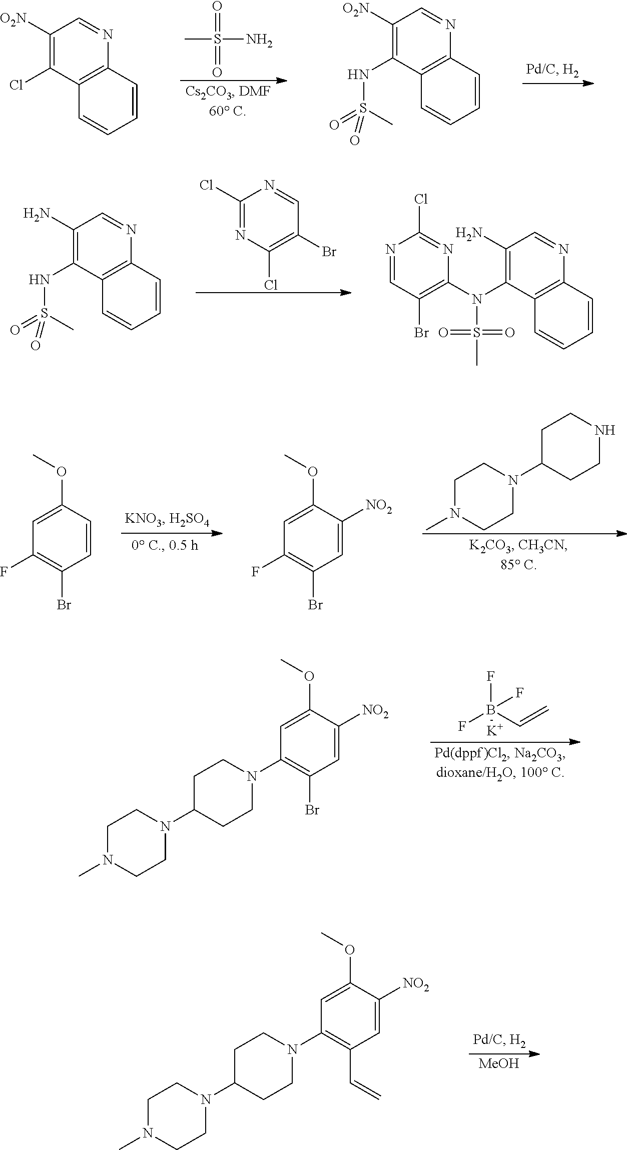

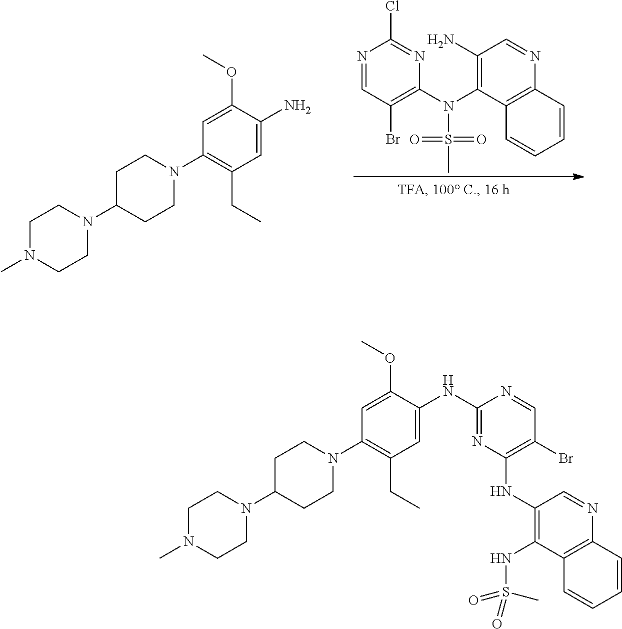 [SMILES: CCc1cc(N)c(OC)cc1N1CCC(N2CCN(C)CC2)CC1 ; CS(=O)(=O)N(c1nc(Cl)ncc1Br)c1c(N)cnc2ccccc12 ; CCc1cc(Nc2ncc(Br)c(Nc3cnc4ccccc4c3NS(C)(=O)=O)n2)c(OC)cc1N1CCC(N2CCN(C)CC2)CC1]

### Step 1: Preparation of N-(3-nitroquinolin-4-yl)methanesulfonamide

A solution of 4-chloro-3-nitroquinoline (2 g, 9.59 mmol), methanesulfonamide (912 mg, 9.59 mmol) and Cs₂CO₃ (4.7 g, 14.38 mmol) in DMF (10 mL) was stirred under nitrogen at 60° C. for 3 h. The reaction was concentrated and purified by Combi-Flash (DCM/MeOH=10/1) to afford N-(3-nitroquinolin-4-yl)methanesulfonamide (3.6 g, 98.3%) as a yellow solid. LC/MS: 267.9 [M+H]⁺.

### Step 2: Preparation of N-(3-aminoquinolin-4-yl)methanesulfonamide

To a solution of N-(3-nitroquinolin-4-yl)methanesulfonamide (3.6 g, 13.47 mmol) in MeOH (50 mL) was added Pd/C (3.6 g). The reaction was stirred under H₂ at 20° C. for 3 h. The mixture was filtered and the filtrate was concentrated to give the crude product (3.6 g) which was used in the next step without further purification. LC/MS: 238.0 [M+H]⁺.

### Step 3: Preparation of N-(3-aminoquinolin-4-yl)-N-(5-bromo-2-chloropyrimidin-4-yl)methanesulfonamide

A mixture of N-(3-aminoquinolin-4-yl)methanesulfonamide (1.5 g, 6.32 mmol), 5-bromo-2,4-dichloropyrimidine (720 mg, 3.16 mmol) and K₂CO₃ (1.7 g, 12.64 mmol) in DMF (15 mL) was stirred under nitrogen at 50° C. for 1 hr. The reaction was concentrated and purified by Combi-Flash (DCM/MeOH=20/1) to afford the desired product (380 mg, 11.2%) as a yellow solid. LC/MS: 429.8 [M+H]⁺.

### Step 4: Preparation of 1-bromo-2-fluoro-4-methoxy-5-nitrobenzene

To a solution of 1-bromo-2-fluoro-4-methoxybenzene (5 g, 0.244 mmol) in H₂SO₄ (6 M, 2 mL), KNO₃ (2.47 g, 0.244 mmol) was added at 0° C. The reaction mixture was stirred at 0° C. for 0.5 hr. The mixture was poured into ice water (50 mL) and extracted with ethyl acetate (100 ml). The organic layer was concentrated in vacuo and purified with flash column chromatography (PE/EA=0-20%) to give 1-[1-(2-bromo-5-methoxy-4-nitrophenyl)piperidin-4-yl]-4-methylpiperazine (2.8 g, 43.4%) as a yellow solid. LC/MS: 250.0[M+H]⁺.

### Step 5: Preparation of 1-[1-(2-bromo-5-methoxy-4-nitrophenyl)piperidin-4-yl]-4-methylpiperazine

To a solution of 1-bromo-2-fluoro-4-methoxy-5-nitrobenzene (1 g, 4 mmol) and 1-methyl-4-(piperidin-4-yl)piperazine (0.81 g, 4.4 mmol) in MeCN (20 mL) was added K₂CO₃ (2.3 g, 5.2 mmol). The reaction mixture was stirred at r.t. for 4 hr. The reaction mixture was concentrated in vacuo and purified with flash column chromatography (DCM/MeOH=0-10%) to give 1-[1-(2-bromo-5-methoxy-4-nitrophenyl)piperidin-4-yl]-4-methylpiperazine (1.4 g, 80%) as a yellow solid. LC/MS: 413.9[M+H]⁺.

### Step 6: Preparation of 1-[1-(2-ethenyl-5-methoxy-4-nitrophenyl)piperidin-4-yl]-4-methylpiperazine

To a solution of 1-[1-(2-bromo-5-methoxy-4-nitrophenyl)piperidin-4-yl]-4-methylpiperazine (1 g, 2.42 mmol) and potassium vinyltrifluoroborate (324 mg, 2.42 mmol) in 1,4-dioxane/H₂O (4/1.10 mL) was added Na₂CO₃ (1 g, 7.26 mmol) and Pd(dppf)Cl₂ (442 mg, 1 mmol). The reaction mixture was stirred at 100° C. for 12 hr under N₂. The reaction mixture was concentrated and purified by combi-flash (DCM/MeOH from 0 to 10%) to give 1-[1-(2-ethenyl-5-methoxy-4-nitrophenyl)piperidin-4-yl]-4-methylpiperazine (530 mg, 61%) as a brown solid. LC/MS: 361.2 [M+H]⁺.

### Step 7: Preparation of 5-ethyl-2-methoxy-4-[4-(4-methylpiperazin-1-yl)piperidin-1-yl]aniline

To a solution of 1-[1-(2-ethenyl-5-methoxy-4-nitrophenyl)piperidin-4-yl]-4-methylpiperazine (200 mg, 0.55 mmol) in MeOH (5 mL) was added Pd/C (200 mg) at rt. The reaction mixture was stirred at r.t. for 2 hr. After filtration, the filtrate was concentrated in vacuo to give 5-ethyl-2-methoxy-4-[4-(4-methylpiperazin-1-yl)piperidin-1-yl]aniline (160 mg, 82.4%) as a brown solid. LC/MS: 333.1 [M+H]⁺.

### Step 8: Preparation of N-(3-{[5-bromo-2-({5-ethyl-2-methoxy-4-[4-(4-methylpiperazin-1-yl)piperidin-1-yl]phenyl}amino)pyrimidin-4-yl]amino}quinolin-4-yl)methanesulfonamide (Compound 15)

To a mixture of 5-ethyl-2-methoxy-4-[4-(4-methylpiperazin-1-yl)piperidin-1-yl]aniline (80 mg, 0.24 mmol) and N-{3-[(5-bromo-2-chloropyrimidin-4-yl)amino]quinolin-4-yl}methanesulfonamide (124 mg, 0.29 mmol) in i-PrOH (1 mL) was added TFA (274 mg, 2.4 mmol). The reaction mixture was stirred at 100° C. for 16 hr. The reaction mixture was concentrated in vacuo and purified by prep-HPLC [H₂O (0.01% TFA)/ACN from 75% to 45% over 30 minutes] to afford the title compound (31 mg, 17%) as a yellow solid. LC/MS: 725.0 [M+H]⁺; ¹H NMR (400 MHz, DMSO) δ 9.04 (s, 1H), 8.93 (s, 1H), 8.44 (s, 2H), 8.26 (s, 1H), 7.85-7.79 (m, 2H), 7.58 (t, J=7.4 Hz, 1H), 7.35 (s, 1H), 6.66 (s, 1H), 3.74 (s, 3H), 3.50-3.52 (m, 4H), 3.06-3.12 (m, 8H), 2.90-2.94 (m, 2H), 2.81 (s, 3H), 2.60-2.63 (m, 2H), 2.01-2.05 (m, 4H), 1.66-1.68 (m, 2H), 0.65-0.61 (m, 3H).

### Example 16: Preparation of N-(6-((5-bromo-2-((5-ethyl-2-methoxy-4-(4-(4-methyl-1,4-diazepan-1-yl)piperidin-1-yl)phenyl)amino)pyrimidin-4-yl)amino)quinoxalin-5-yl)methanesulfonamide (Compound 17)

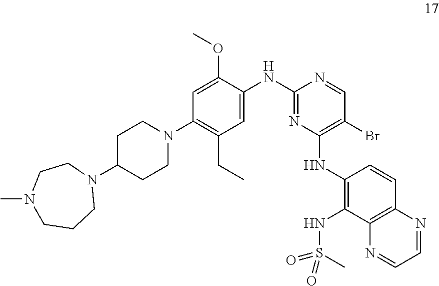 [SMILES: CCc1cc(Nc2ncc(Br)c(Nc3ccc4nccnc4c3NS(C)(=O)=O)n2)c(OC)cc1N1CCC(N2CCCN(C)CC2)CC1]

Compound 17 was prepared analogously with the procedure described for Compound 15. LC/MS: 738.6[M+H]⁺. ¹H NMR (400 MHz, CD₃OD) δ 9.02-9.04 (m, 1H), 8.90-8.92 (m, 1H), 8.54-8.56 (m, 1H), 8.27-8.29 (m, 1H), 7.98-8.00 (m, 1H), 7.53-7.55 (m, 1H), 6.82-6.84 (m, 1H), 3.86-3.88 (m, 2H), 3.72-3.76 (m, 2H), 3.53-3.56 (m, 3H), 3.20-3.22 (m, 2H), 3.01-3.03 (m, 3H), 2.88-2.90 (m, 2H), 2.44-2.46 (m, 2H), 2.32-2.36 (m, 2H), 2.19-2.21 (m, 3H), 2.06-2.08 (m, 2H), 1.96-1.98 (m, 1H), 1.29-1.38 (m, 9H).

### Example 17: Preparation of N-(6-((2-((4-(4-(1,4-oxazepan-4-yl)piperidin-1-yl)-5-ethyl-2-methoxyphenyl)amino)-5-bromopyrimidin-4-yl)amino)quinoxalin-5-yl)methanesulfonamide (Compound 19)

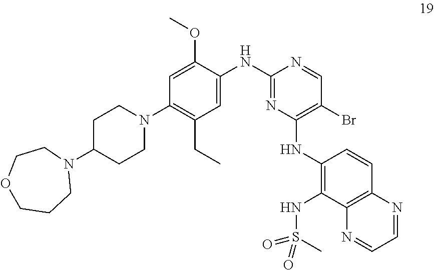 [SMILES: CCc1cc(Nc2ncc(Br)c(Nc3ccc4nccnc4c3NS(C)(=O)=O)n2)c(OC)cc1N1CCC(N2CCCOCC2)CC1]

Compound 19 was prepared analogously with the procedure described for Compound 15. LC/MS: 725.7[M+H]⁺.

¹H NMR (400 MHz, MeOH,) δ 8.96-8.98 (m, 1H), 8.90-8.92 (m, 1H), 8.42-8.45 (m, 1H), 8.24 (s, 1H), 7.94 (dd, J=8.6, 0.5 Hz, 1H), 7.35 (s, 1H), 6.79 (s, 1H), 3.90-3.94 (m, 3H), 3.72-3.79 (m, 4H), 3.50-3.58 (m, 5H), 3.16-3.19 (m, 2H), 2.96-2.98 (m, 3H), 2.82-2.85 (m, 2H), 2.42-2.38 (m, 2H), 2.15-2.11 (m, 4H), 1.96-1.98 (m, 2H), 0.80-0.82 (m, 3H).

### Example 18: Preparation of N-[6-[[2-[5-ethyl-2-methoxy-4-[4-(4-methylpiperazin-1-yl)-1-piperidyl]anilino]-5-(trifluoromethyl)pyrimidin-4-yl]amino]quinoxalin-5-yl]methanesulfonamide (Compound 26)

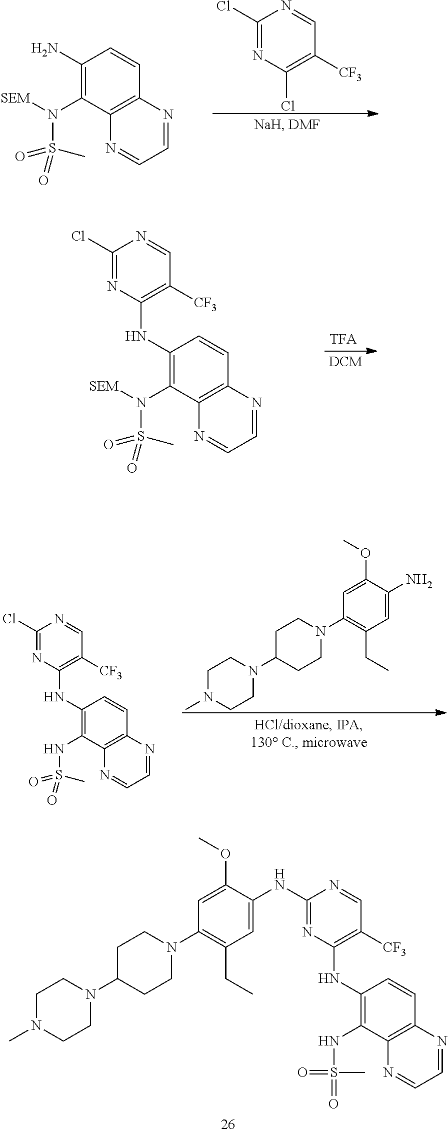 [SMILES: C[Si](C)(C)CCOCN(c1c(N)ccc2nccnc12)S(C)(=O)=O ; FC(F)(F)c1cnc(Cl)nc1Cl ; C[Si](C)(C)CCOCN(c1c(Nc2nc(Cl)ncc2C(F)(F)F)ccc2nccnc12)S(C)(=O)=O ; CS(=O)(=O)Nc1c(Nc2nc(Cl)ncc2C(F)(F)F)ccc2nccnc12 ; CCc1cc(N)c(OC)cc1N1CCC(N2CCN(C)CC2)CC1 ; CCc1cc(Nc2ncc(C(F)(F)F)c(Nc3ccc4nccnc4c3NS(C)(=O)=O)n2)c(OC)cc1N1CCC(N2CCN(C)CC2)CC1]

### Step 1: Preparation of N-[6-[[2-chloro-5-(trifluoromethyl)pyrimidin-4-yl]amino]quinoxalin-5-yl]-N-(2-trimethylsilylethoxymethyl)methanesulfonamide

To a solution of N-(6-aminoquinoxalin-5-yl)-N-(2-trimethylsilylethoxymethyl)methanesulfonamide (300 mg, 814 umol) in DMF (10 mL) was added NaH (65.1 mg, 1.63 mmol, 60% purity) at 25° C. After stirring for 0.5 hr, 2,4-dichloro-5-(trifluoromethyl)pyrimidine (212 mg, 977 umol) was added. The reaction was stirred at 25° C. for 1.5 hr. The reaction was quenched by adding saturated NH₄Cl aqueous (10 mL) and extracted with EtOAc (20 mL×2). The combined organic phase was washed with brine (20 mL), dried over Na₂SO₄, filtered and concentrated. The residue was purified by prep-HPLC(column: Phenomenex Gemini-NX C18 75×30 mm×3 um; mobile phase: [water(0.225% FA)-ACN];B %: 58%-88%, 7 min) to give the product (100 mg, 22% yield) as a black brown solid. LC/MS: 548.9 [M+H]⁺. ¹H NMR (400 MHz, CDCl₃-d) δ=9.17 (br s, 1H), 8.89 (s, 1H), 8.85 (d, J=1.6 Hz, 1H), 8.61-8.43 (m, 2H), 8.22 (d, J=8.8 Hz, 1H), 5.41 (d, J=10.0 Hz, 1H), 4.98 (d, J=10.8 Hz, 1H), 3.68-3.60 (m, 1H), 3.56-3.48 (m, 1H), 3.02 (s, 3H), 0.82-0.66 (m, 2H), −0.09 (s, 9H).

### Step 2: Preparation of N-[6-[[2-chloro-5-(trifluoromethyl)pyrimidin-4-yl]amino]quinoxalin-5-yl]methanesulfonamide

To a solution of N-[6-[[2-chloro-5-(trifluoromethyl)pyrimidin-4-yl]amino]quinoxalin-5-yl]-N-(2-trimethylsilylethoxymethyl)methanesulfonamide (100 mg, 182 umol) in DCM (3 mL) was added TFA (1 mL). The reaction was stirred at 25° C. for 5 hr. The reaction was concentrated. H₂O (20 mL) was added and the aqueous phase was extracted with EtOAc (20 mL×2). The combined organic phase was washed with brine (20 mL), dried over Na₂SO₄, filtered and concentrated to give the product (100 mg) as a black brown solid. LC/MS: 419.1 [M+H]⁺.

### Step 3: Preparation of N-[6-[[2-[5-ethyl-2-methoxy-4-[4-(4-methylpiperazin-1-yl)-1-piperidyl]anilino]-5-(trifluoromethyl)pyrimidin-4-yl]amino]quinoxalin-5-yl]methanesulfonamide (26)

To a solution of N-[6-[[2-chloro-5-(trifluoromethyl)pyrimidin-4-yl]amino]quinoxalin-5-yl]methanesulfonamide (100 mg, 239 umol), 5-ethyl-2-methoxy-4-[4-(4-methylpiperazin-1-yl)-1-piperidyl]aniline (111 mg, 334 umol) in IPA (2 mL) was added HCl/dioxane (4 M, 0.30 mL). The reaction was stirred at 130° C. for 4 hr under microwave irradiation. The reaction mixture was concentrated. The residue was purified by prep-HPLC (column: Waters Xbridge C18 150×50 mm×10 um; mobile phase: [water(10 mM NH₄HCO₃)-ACN];B %: 41%-71%, 11 min) to give the product (9.22 mg, 5.2% yield) as a yellow solid. LC/MS: 715.4 [M+H]⁺. ¹H NMR (400 MHz, CDCl₃-d) δ=9.03 (s, 1H), 8.93 (d, J=2.0 Hz, 1H), 8.86 (d, J=2.0 Hz, 1H), 8.50 (d, J=9.2 Hz, 1H), 8.41 (s, 1H), 8.03 (d, J=9.2 Hz, 1H), 7.80 (br s, 1H), 7.63 (br s, 1H), 6.61 (s, 1H), 3.84 (s, 3H), 3.04 (d, J=11.2 Hz, 2H), 2.94 (s, 3H), 2.84-2.59 (m, 12H), 2.42 (s, 4H), 1.97-1.91 (m, 2H), 1.73-1.67 (m, 2H), 1.40-1.30 (m, 3H).

### Example 19: Preparation of N-(6-((5-bromo-2-((5-ethyl-2-methoxy-4-(4-(4-methylpiperazin-1-yl)piperidin-1-yl)phenyl)amino)pyrimidin-4-yl)amino)quinoxalin-5-yl)ethanesulfonamide (Compound 39)

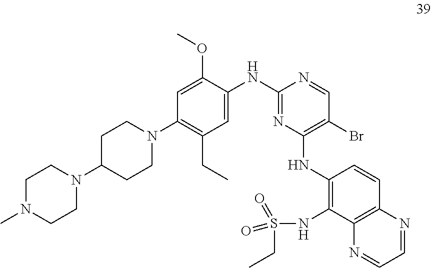 [SMILES: CCc1cc(Nc2ncc(Br)c(Nc3ccc4nccnc4c3NS(=O)(=O)CC)n2)c(OC)cc1N1CCC(N2CCN(C)CC2)CC1]

Compound 39 was prepared analogously with the procedure described for Compound 2. LC/MS: 741.4[M+H]⁺. ¹H NMR (400 MHz, CDCl₃-d) δ=9.19 (s, 1H), 8.90 (d, J=1.6 Hz, 1H), 8.83 (d, J=1.6 Hz, 1H), 8.65 (d, J=9.2 Hz, 1H), 8.26 (s, 1H), 8.03 (d, J=9.2 Hz, 1H), 7.87 (s, 1H), 7.40 (s, 1H), 6.62 (s, 1H), 3.83 (s, 3H), 3.10-3.02 (m, 4H), 2.86-2.58 (m, 1 OH), 2.44-2.35 (m, 6H), 1.96-1.90 (m, 2H), 1.72-1.66 (m, 2H), 1.43 (t, J=7.2 Hz, 3H), 0.71 (s, 3H).

### Example 20: Preparation of N-[6-[[5-bromo-2-[5-ethyl-2-methoxy-4-[4-(4-methylpiperazin-1-yl)-1-piperidyl]anilino]pyrimidin-4-yl]amino]quinoxalin-5-yl]cyclopropanesulfonamide (Compound 40)

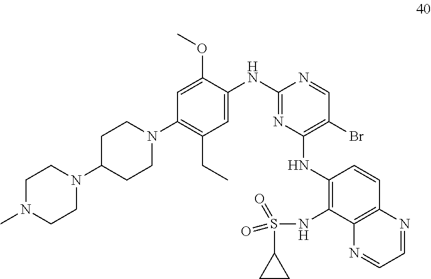 [SMILES: CCc1cc(Nc2ncc(Br)c(Nc3ccc4nccnc4c3NS(=O)(=O)C3CC3)n2)c(OC)cc1N1CCC(N2CCN(C)CC2)CC1]

Compound 40 was prepared analogously with the procedure described for Compound 2. LC/MS: 753.2 [M+H]⁺. ¹H NMR (400 MHz, CDCl₃-d) δ=9.13 (s, 1H), 8.89 (d, J=1.6 Hz, 1H), 8.83 (d, J=2.0 Hz, 1H), 8.69 (d, J=9.2 Hz, 1H), 8.25 (s, 1H), 8.03 (d, J=9.2 Hz, 1H), 7.89 (s, 1H), 7.41 (s, 1H), 6.63 (s, 1H), 3.84 (s, 3H), 3.05 (d, J=11.2 Hz, 2H), 2.79-2.52 (m, 9H), 2.47-2.32 (m, 8H), 1.97-1.90 (m, 2H), 1.74-1.65 (m, 2H), 1.07-0.99 (m, 2H), 0.81-0.64 (m, 5H).

### Example 21: Preparation of N-(6-((5-bromo-2-((5-ethyl-2-(methoxy-d3)-4-(4-(4-methylpiperazin-1-yl)piperidin-1-yl)phenyl)amino)pyrimidin-4-yl)amino)quinoxalin-5-yl)methanesulfonamide (Compound 41)

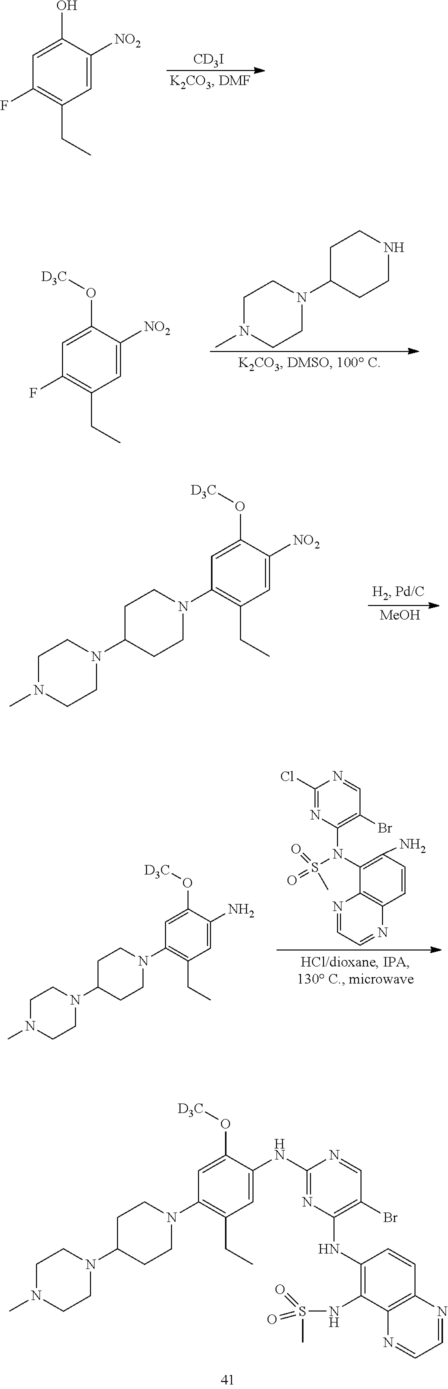

### Step 1: Preparation of 1-ethyl-2-fluoro-4-(methoxy-d3)-5-nitrobenzene

To a solution of 4-ethyl-5-fluoro-2-nitro-phenol (1 g, 5.40 mmol) in DMF (7 mL) was added K₂CO₃ (1.12 g, 8.10 mmol), followed by trideuterio(iodo)methane (1.02 g, 7.02 mmol). The mixture was stirred at 30° C. for 12 h. The reaction mixture was added dropwise to water (100 mL) with stirring. The precipitate formed was filtered and collected. The wet cake was dried under reduced pressure to give product (1.05 g, 96% yield) as a yellow solid. ¹H NMR (400 MHz, DMSO-d₆) δ=7.92 (d, J=7.6 Hz, 1H), 7.27 (dd, J=2.4, 12.0 Hz, 1H), 2.60 (q, J=7.6 Hz, 2H), 1.15 (t, J=7.6 Hz, 3H).

### Step 2: Preparation of 1-(1-(2-ethyl-5-(methoxy-d3)-4-nitrophenyl)piperidin-4-yl)-4-methylpiperazine

To a solution of 1-ethyl-2-fluoro-5-nitro-4-(trideuteriomethoxy)benzene (1.05 g, 5.19 mmol) in DMSO (10 mL) was added K₂CO₃ (1.08 g, 7.79 mmol) followed by 1-methyl-4-(4-piperidyl)piperazine (1.14 g, 6.23 mmol). The solution was stirred at 100° C. for 12 h. The solution was diluted with water (150 mL) and the mixture was extracted with dichloromethane/methanol (10/1; 70 mL×3). The combined organic layers were washed with brine (50 mL), dried over sodium sulfate, filtered and concentrated to give a yellow solid. The yellow solid was triturated with methyl t-butylether (5 mL) to give the product (1.33 g, 68% yield) as a yellow solid. LCMS: 366.3 [M+H]⁺. ¹H NMR (400 MHz, DMSO-d₆) δ=7.76 (s, 1H), 6.74 (s, 1H), 3.32-3.29 (m, 2H), 3.25 (d, J=12.4 Hz, 2H), 2.73 (t, J=12.0 Hz, 2H), 2.63-2.51 (m, 5H), 2.38-2.25 (m, 4H), 1.86 (d, J=11.6 Hz, 2H), 1.56 (dq, J=3.6, 12.0 Hz, 2H), 1.19 (t, J=7.6 Hz, 3H).

### Step 3: Preparation of 5-ethyl-2-(methoxy-d3)-4-(4-(4-methylpiperazin-1-yl)piperidin-1-yl)aniline

To a solution of 1-[1-[2-ethyl-4-nitro-5-(trideuteriomethoxy)phenyl]-4-piperidyl]-4-methyl-piperazine (400 mg, 1.07 mmol) in MeOH (20 mL) was added Pd/C (50 mg). The suspension was degassed and refilled with hydrogen for three times. The reaction mixture was stirred at 20° C. for 1 h under H₂ balloon (15 psi). The solution was filtered through a celite pad and the filtrate was concentrated to give product (360 mg, 95% yield) as a yellow gum. LCMS: 336.4 [M+H]⁺.

### Step 4: Preparation of N-(6-((5-bromo-2-((5-ethyl-2-(methoxy-d3)-4-(4-(4-methylpiperazin-1-yl)piperidin-1-yl)phenyl)amino)pyrimidin-4-yl)amino)quinoxalin-5-yl)methanesulfonamide (41)

To a solution of 5-ethyl-4-[4-(4-methylpiperazin-1-yl)-1-piperidyl]-2-(trideuteriomethoxy)aniline (105 mg, 302 umol) in IPA (20 mL) was added N-(6-aminoquinoxalin-5-yl)-N-(5-bromo-2-chloro-pyrimidin-4-yl)methanesulfonamide (100 mg, 232 umol), followed by HCl/dioxane (4 M, 0.3 mL). The solution was stirred under microwave irradiation at 130° C. for 4 h. The solution was concentrated. The residue was purified by prep-HPLC (column: Phenomenex luna C18 150×40 mm×15 um; mobile phase: [water(0.1% TFA)-ACN]; B %: 8%-38%, 11 min) and the desired fraction was concentrated to remove organic solvent. The remained aqueous part was adjusted to pH=8 by addition of sodium bicarbonate solid and then extracted with dichloromethane/methanol (10/1; 30 mL×3). The combined organic layers were washed with brine (50 mL), dried over sodium sulfate, filtered and concentrated. The residue was purified by prep-TLC (dichloromethane/methanol: 7/1) to give a yellow solid. The yellow solid was purified by prep-HPLC(column: Waters Xbridge 150×25 mm×5 um; mobile phase: [water(10 mM NH₄HCOs)-ACN];B %: 39%-69%, 10 min) to give N-[6-[[5-bromo-2-[5-ethyl-4-[4-(4-methylpiperazin-1-yl)-1-piperidyl]-2-(trideuteriomethoxy)anilino]pyrimidin-4-yl]amino]quinoxalin-5-yl]methanesulfonamide (103.18 mg, 58% yield) as a yellow solid. LCMS: 730.1 [M+H]⁺. ¹H NMR (400 MHz, CDCl₃-d) δ=9.12 (s, 1H), 8.90 (s, 1H), 8.84 (d, J=1.6 Hz, 1H), 8.73 (d, J=9.6 Hz, 1H), 8.26 (s, 1H), 8.05 (d, J=9.6 Hz, 1H), 7.88 (s, 1H), 7.40 (s, 1H), 6.62 (s, 1H), 3.04 (d, J=11.2 Hz, 2H), 2.95 (s, 3H), 2.69-2.47 (m, 10H), 2.43-2.32 (m, 6H), 1.90 (d, J=11.2 Hz, 2H), 1.67 (d, J=11.6 Hz, 2H), 0.80-0.70 (m, 3H).

### Example 22: Preparation of N-(6-((5-bromo-2-((2-methoxy-5-(1-methyl-1H-pyrazol-4-yl)-4-(4-(4-methylpiperazin-1-yl)piperidin-1-yl)phenyl)amino)pyrimidin-4-yl)amino)quinoxalin-5-yl)methanesulfonamide (Compound 48)

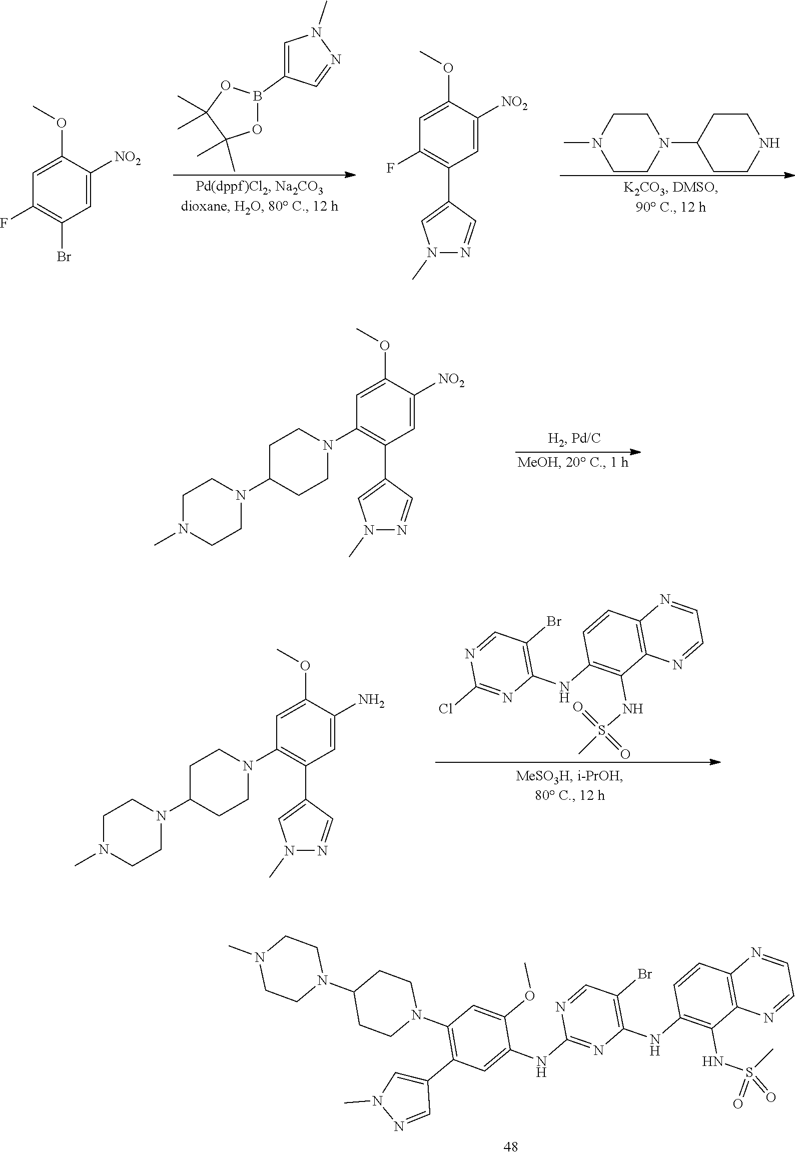

### Step 1: Preparation of 4-(2-fluoro-4-methoxy-5-nitrophenyl)-1-methyl-1H-pyrazole

A mixture of 1-bromo-2-fluoro-4-methoxy-5-nitro-benzene (860 mg, 3.44 mmol), 1-methyl-4-(4,4,5,5-tetramethyl-1,3,2-dioxaborolan-2-yl)pyrazole (850 mg, 4.09 mmol), Pd(dppf)Cl₂·CH₂Cl₂ (281 mg, 344 umol) and Na₂CO₃ (1.09 g, 10.3 mmol) in dioxane (15 mL) and water (1.5 mL) was degassed and purged with N₂ for 3 times, and the mixture was stirred at 80° C. for 12 hr under N₂ atmosphere. The reaction mixture was concentrated under vacuum and the residue was purified by column chromatography (Petroleum ether/Ethyl acetate=10/1 to 0/1) to give product (840 mg, 97% yield) as a yellow solid. LC/MS: 252.1 [M+H]⁺. ¹H NMR (400 MHz, CDCl₃) δ=8.15 (d, J=7.6 Hz, 1H), 7.83 (s, 1H), 7.75 (d, J=2.0 Hz, 1H), 6.87 (d, J=12.0 Hz, 1H), 3.98 (s, 6H)

### Step 2: Preparation of 1-(1-(5-methoxy-2-(1-methyl-1H-pyrazol-4-yl)-4-nitrophenyl)piperidin-4-yl)-4-methylpiperazine

To a solution of 4-(2-fluoro-4-methoxy-5-nitro-phenyl)-1-methyl-pyrazole (840 mg, 3.34 mmol) and 1-methyl-4-(4-piperidyl)piperazine (613 mg, 3.34 mmol) in DMSO (10 mL) was added K₂CO₃ (1 g, 7.24 mmol). The mixture was stirred at 90° C. for 12 hr. The reaction mixture was filtered. The filtrate was purified by reversed-phase HPLC (0.1% NH₃—H₂O; Column: 330 g Flash Column Welch Ultimate XB_C18 20-40 μm; 120 A; Solvent for sample dissolution 1.50 grams of sample dissolved in 10 ml of DMSO; Flow rate: 100 ml/min; Mobile phase: MeCN/H₂O; Instrument: TELEDYNE ISCO CombiFlashRf150) to give the titled product (460 mg, 31% yield) as a yellow gum. LC/MS: 415.3 [M+H]⁺. ¹H NMR (400 MHz, DMSO-d₆) δ=8.10 (s, 1H), 7.90-7.79 (m, 2H), 6.76 (s, 1H), 3.94 (s, 3H), 3.87 (s, 3H), 3.32-3.29 (m, 2H), 2.67-2.52 (m, 5H), 2.35-2.17 (m, 6H), 2.14 (s, 3H), 1.82-1.73 (m, 2H), 1.57-1.43 (m, 2H).

### Step 3: Preparation of 2-methoxy-5-(1-methyl-1H-pyrazol-4-yl)-4-(4-(4-methylpiperazin-1-yl)piperidin-1-yl)aniline

To a solution of 1-[1-[5-methoxy-2-(1-methylpyrazol-4-yl)-4-nitro-phenyl]-4-piperidyl]-4-methyl-piperazine (260 mg, 627 umol) in MeOH (20 mL) was added Pd/C (100 mg, 10% on carbon). The mixture was stirred at 20° C. for 1 hr under H₂ (15 Psi). The reaction mixture was filtered and the filtrate was concentrated under reduced pressure to give the titled product (240 mg, crude) as a yellow oil which was used in the next step without further purification. LC/MS: 385.3 [M+H]⁺.

### Step 4: Preparation of N-(6-((5-bromo-2-((2-methoxy-5-(1-methyl-1H-pyrazol-4-yl)-4-(4-(4-methylpiperazin-1-yl)piperidin-1-yl)phenyl)amino)pyrimidin-4-yl)amino)quinoxalin-5-yl)methanesulfonamide (48)

To a solution of N-[6-[(5-bromo-2-chloro-pyrimidin-4-yl)amino]quinoxalin-5-yl]methanesulfonamide (200 mg, 465 umol) in IPA (12 mL) was added methanesulfonic acid (223 mg, 2.33 mmol) and 2-methoxy-4-[4-(4-methylpiperazin-1-yl)-1-piperidyl]-5-(1-methylpyrazol-4-yl)aniline (200 mg, 520 umol). The mixture was stirred at 80° C. for 12 hr. The reaction mixture was concentrated under reduced pressure to dryness. The crude product was purified by prep-HPLC (column: Phenomenex Synergi C18 150×25 mm×10 um; mobile phase: [water(FA)-ACN]; B %: 8%-38%, 10 min). Pure fraction was adjusted with saturated aqueous solution of NaHCO₃ to pH=7, and extracted with CHCl₃/MeOH (10/1, 50 mL×2). The combined organic layers were dried over Na₂SO₄, filtered and concentrated. The residue was purified by prep-TLC (DCM:MeOH=5:1) to give product (78.59 mg, 21% yield) as a yellow solid. LC/MS: 777.1 [M+H]⁺. ¹H NMR (400 MHz, CDCl₃) δ=9.10 (s, 1H), 8.83 (dd, J=2.0, 10.8 Hz, 2H), 8.71 (d, J=9.6 Hz, 1H), 8.27 (s, 1H), 8.14 (s, 1H), 7.78 (s, 1H), 7.53 (br s, 1H), 7.31 (s, 2H), 6.67 (s, 1H), 3.87 (s, 3H), 3.79 (s, 3H), 3.17 (d, J=11.6 Hz, 2H), 2.93 (s, 3H), 2.80-2.47 (m, 10H), 2.36 (s, 3H), 2.29-2.23 (m, 1H), 1.97-1.90 (m, 2H), 1.66-1.55 (m, 2H).

### Example 23: Synthesis of N-(6-((5-bromo-2-((5-ethyl-2-methoxy-4-((3aR,6aS)-5-methylhexahydropyrrolo[3,4-c]pyrrol-2(1H)-yl)phenyl)amino)pyrimidin-4-yl)amino)quinoxalin-5-yl)methanesulfonamide (Compound 67)

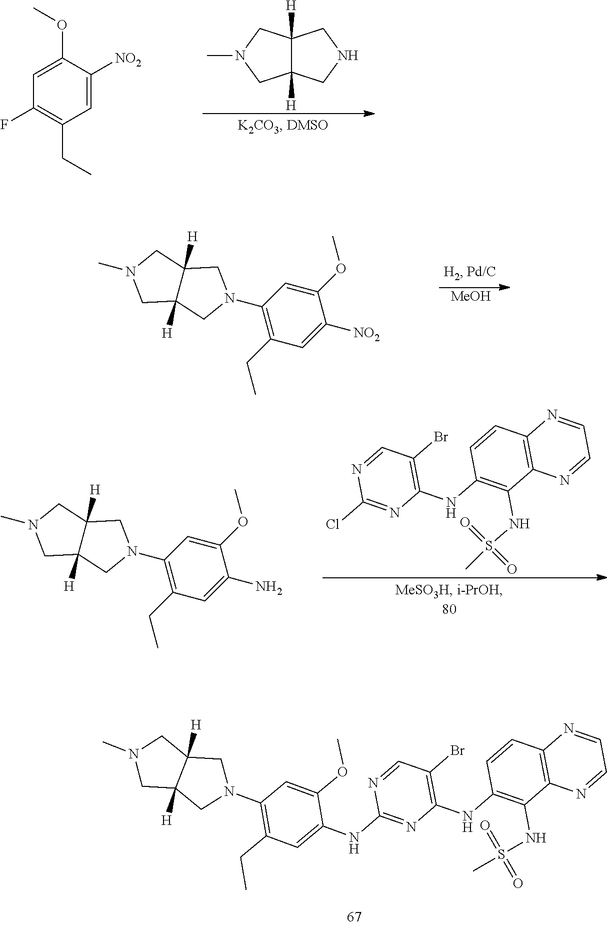

### Step 1: Preparation of (3aR,6aS)-2-(2-ethyl-5-methoxy-4-nitrophenyl)-5-methyloctahydropyrrolo[3,4-c]pyrrole

To a solution of 1-ethyl-2-fluoro-4-methoxy-5-nitro-benzene (200 mg, 1.00 mmol) in DMSO (4 mL) was added K₂CO₃ (416 mg, 3.01 mmol) and (3aR,6aS)-5-methyl-2,3,3a,4,6,6a-hexahydro-1H-pyrrolo[3,4-c]pyrrole (140 mg, 1.11 mmol). The mixture was stirred at 90° C. for 12 hr. The reaction mixture was partitioned between H₂O (50 mL) and EtOAc (50 mL). The organic phase was separated, washed with brine (30 mL), dried over Na₂SO₄, filtered and concentrated under reduced pressure to give product (300 mg, 98% yield) as yellow oil. ¹H NMR (400 MHz, CDCl₃-d) δ=7.87 (s, 1H), 6.44 (s, 1H), 3.94 (s, 3H), 3.38-3.33 (m, 2H), 3.08 (dd, J=4.0, 9.6 Hz, 2H), 2.99-2.90 (m, 2H), 2.74-2.70 (m, 2H), 2.67-2.63 (m, 2H), 2.45 (dd, J=3.6, 9.2 Hz, 2H), 2.36 (s, 3H), 1.27 (t, J=7.6 Hz, 3H).

### Step 2: Preparation of 5-ethyl-2-methoxy-4-((3aR,6aS)-5-methylhexahydropyrrolo[3,4-c]pyrrol-2(1H)-yl)aniline

To a solution of (3aS,6aR)-5-(2-ethyl-5-methoxy-4-nitro-phenyl)-2-methyl-1,3,3a,4,6,6a-hexahydropyrrolo[3,4-c]pyrrole (300 mg, 982 umol) in MeOH (20 mL) was added Pd/C (10%, 200 mg) under N₂ atmosphere. The suspension was degassed and purged with H₂ for 3 times. The mixture was stirred under H₂ (15 Psi) at 25° C. for 1 hr. The reaction mixture was filtered and the filtrate was concentrated to give product (240 mg, 88% yield) as a purple oil. ¹H NMR (400 MHz, CDCl₃-d) δ=6.66 (s, 1H), 6.59 (s, 1H), 3.82 (s, 3H), 3.58 (br s, 2H), 2.98-2.88 (m, 4H), 2.85-2.78 (m, 4H), 2.60 (d, J=7.6 Hz, 2H), 2.37 (s, 3H), 2.29-2.26 (m, 2H), 1.20 (t, J=7.6 Hz, 3H).

### Step 3: Preparation of N-(6-((5-bromo-2-((5-ethyl-2-methoxy-4-((3aR,6aS)-5-methylhexahydropyrrolo[3,4-c]pyrrol-2(1H)-yl)phenyl)amino)pyrimidin-4-yl)amino)quinoxalin-5-yl)methanesulfonamide (67)

To a solution of 4-[(3aS,6aR)-2-methyl-1,3,3a,4,6,6a-hexahydropyrrolo[3,4-c]pyrrol-5-yl]-5-ethyl-2-methoxy-aniline (80 mg, 290 umol) in IPA (4 mL) was added methanesulfonic acid (135 mg, 1.40 mmol, 6.04 eq) and N-[6-[(5-bromo-2-chloro-pyrimidin-4-yl)amino]quinoxalin-5-yl]methanesulfonamide (100 mg, 232 umol). The mixture was stirred at 80° C. for 12 hr. The reaction mixture was concentrated under vacuum to get a residue. The residue was purified by prep-HPLC (column: Phenomenex Synergi C18 150×25 mm×10 um; mobile phase: [water(FA)-ACN];B %: 12%-42%, 10 min) to give product (57.53 mg, 33% yield, 97.26% purity, FA salt) as a yellow solid. LC/MS: 670.1[M+H]⁺. ¹H NMR (400 MHz, CDCl₃-d) δ=9.13 (s, 1H), 8.90 (d, J=2.0 Hz, 1H), 8.85 (d, J=2.0 Hz, 1H), 8.73 (d, J=9.2 Hz, 1H), 8.58 (s, 0.36H), 8.26 (s, 1H), 8.05 (d, J=9.2 Hz, 1H), 7.89 (s, 1H), 7.40 (s, 1H), 6.66 (s, 1H), 3.84 (s, 3H), 3.25 (s, 2H), 2.99-2.87 (m, 10H), 2.52 (s, 3H), 2.45-2.40 (m, 3H), 0.80 (s, 3H).

### Example 24: Preparation of N-[6-[[5-bromo-2-[2-methoxy-5-methyl-4-(4-methylpiperazin-1-yl)anilino]pyrimidin-4-yl]amino]quinoxalin-5-yl]methanesulfonamide (Compound 68)

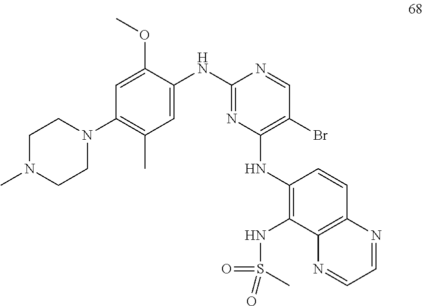 [SMILES: COc1cc(N2CCN(C)CC2)c(C)cc1Nc1ncc(Br)c(Nc2ccc3nccnc3c2NS(C)(=O)=O)n1]

Compound 68 was prepared analogously with the procedure described for compound 1. LC/MS: 630.0 [M+H]⁺. ¹H NMR (400 MHz, CDCl₃-d) δ=9.14 (s, 1H), 8.88 (d, J=1.6 Hz, 1H), 8.84 (d, J=2.0 Hz, 1H), 8.76 (d, J=9.2 Hz, 1H), 8.24 (s, 1H), 8.05 (d, J=9.2 Hz, 1H), 7.82 (s, 1H), 7.41 (s, 1H), 6.58 (s, 1H), 3.82 (s, 3H), 2.95 (s, 3H), 2.88 (t, J=9.2 Hz, 4H), 2.69-2.48 (m, 4H), 2.39 (s, 3H), 1.98 (s, 3H).

### Example 25: Preparation of N-[6-[[2-[5-ethyl-2-methoxy-4-[4-(4-methylpiperazin-1-yl)-1-piperidyl]anilino]-5-methyl-pyrimidin-4-yl]amino]quinoxalin-5-yl]methanesulfonamide (Compound 69)

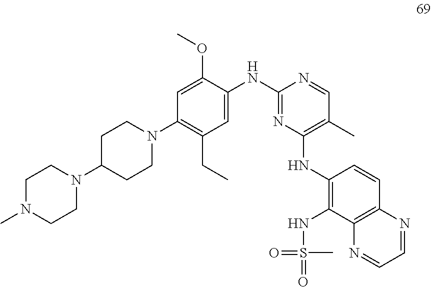 [SMILES: CCc1cc(Nc2ncc(C)c(Nc3ccc4nccnc4c3NS(C)(=O)=O)n2)c(OC)cc1N1CCC(N2CCN(C)CC2)CC1]

Compound 69 was prepared analogously with the procedure described for compound 2. LC/MS: 661.2 [M+H]⁺. ¹H NMR (400 MHz, CDCl₃-d) δ=8.89 (d, J=9.2 Hz, 1H), 8.87-8.79 (m, 2H), 8.70 (s, 1H), 8.07-7.99 (m, 3H), 7.33 (s, 1H), 6.64 (s, 1H), 3.84 (s, 3H), 3.07-3.04 (m, 2H), 2.93 (s, 3H), 2.74-2.55 (m, 10H), 2.47 (q, J=7.6 Hz, 2H), 2.40-2.35 (m, 4H), 2.25 (s, 3H), 1.95-1.90 (m, 2H), 1.72-1.65 (m, 2H), 0.81 (t, J=7.6 Hz, 3H).

### Testing of Compounds for Activity

### Assays

1. Preparation of HCC827-LTC and HCC827-DTC Cell Lines

HCC827 cells (ATCC) were transduced with lentiviral particles expressing codon-optimized human EGFR L858R/T790M/C797S (LTC) mutant or del19/T790M/C797S (DTC) mutant, and then selecting under 5 ug/ml purimycin treatment for a week to generate HCC827 cells stably expressing EGFR LTC or DTC mutants. Next, endogenous EGFR gene in purimycin-selected HCC827 LTC or DTC cells was knocked out using CRISPR-Cas9 technology to ensure the growth of engineered HCC827 cells was solely dependent on exogenous EGFR LTC or DTC mutants. For the EGFR knockout, gRNA complex was prepared by combining Alt-R CRISPR-Cas9 tracrRNA and EGFR crRNA (5′-GCGACCCUCCGGGACGGCCG-3′ for LTC) or (5′-GAUGCUCUCCACGUUGCACA-3′ for DTC) (IDT-Integrated DNA Technologies), and then EGFR ribonucleoprotein (RNP) complex was prepared by combining gRNA complex and Alt-R S.p. Cas9 Nuclease V3 (IDT-Integrated DNA Technologies). Purimycin-selected HCC827 LTC or DTC cells were then electroporated with EGFR RNP complexes using Neon Transfection System (Thermo Fisher). Single clones of HCC827-LTC or HCC827-DTC with endogenous EGFR knockout were picked one week after electroporation and their EGFR knockout was validated by genomic DNA sequencing. HCC827-LTC or HCC827-DTC single clones were also further validated with loss of inhibitory activity by covalent EGFR inhibitors.

2. Cell Growth Inhibition for HCC827, HCC827-LTC, HCC827-DTC and NCI-H2073.

HCC827(ATCC), HCC827-LTC, HCC827-DTC and NCI-H2073 (ATCC) were seeded in 96-well plates at 5000 cells/well in 90 μL of RPMI growth medium containing 10% FBS and 1% Penicillin Streptomycin. Cells were incubated at 37° C. overnight. The following day, the test compound was administered to the cells by using 10× compound stock solution prepared in growth medium at various concentrations. After administration of the compound, cells were then incubated at 37° C. for 3 days. Before CellTiter-Glo assay, the plates were equilibrated at room temperature for approximately 10 minutes. 100 ul of CellTiter-Glo® Reagent (Promega) was added to each well. The plates were then incubated at room temperature for 10 minutes and luminescence was recorded by EnSpire plate reader (PerkinElmer).

### Results

Table 3 illustrates growth inhibition (G150) by exemplary compounds of the present disclosure in multiple cell lines 3 days after administration.

The G150 values (i.e., the concentration of test compound at which 50% of cell growth are inhibited) were calculated. A designation of “A” corresponds to a DC50 value less than 30 nM. A designation of “B” corresponds to a DC50 value greater than or equal to 30 nM and less than 100 nM. A designation of “C” corresponds to a DC50 value greater than or equal to 100 nM and less than 300 nM. A designation of “D” corresponds to a DC50 value greater than or equal to 300 nM.

TABLE 3
Cell Growth Inhibition of the Compounds of the Present Disclosure

| | GI50 (nM). A <30; B ≥30 and <100; | | | |
|---|---|---|---|---|
| | C ≥100 and <300; D ≥300 | | | |
| Compound # | HCC827 | HCC827-LTC | HCC827-DTC | NCI-H2073 |
| 1 | B | C | C | C |
| 2 | A | B | B | C |
| 3 | B | C | C | C |
| 4 | A | C | B | B |

1. A compound of Formula (I) or a tautomer, stereoisomer or a mixture of stereoisomers, or a pharmaceutically acceptable salt, or hydrate, or deuterated derivative thereof: 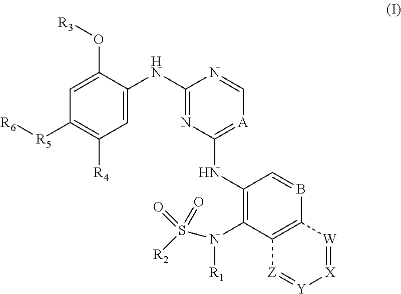
wherein:
W is CH or N;
X is CH or N;
Y is CH or N;
Z is CH or N;
A is N or C—R^{a}, wherein R^{a} is chosen from the group of H, CF₃, halogen, C₁-C₄alkyl, C₃-C₅cycloalkyl, —COOH, and —C(O)OC₁-C₄alkyl, with the proviso that when A is N, the ring containing W, X, Y, and Z may be null;
B is CH or N;
R₁ is H or C₁-C₄alkyl;
R₂ is chosen from —NH₂, C₁-C₄alkyl, and C₃-C₆cycloalkyl;
R₃ is C₁-C₄alkyl, wherein one or more hydrogen atoms are optionally and independently replaced by deuterium or F;
R₄ is chosen from H, C₁-C₄alkyl, C₃-C₅cycloalkyl, vinyl, C₂-alkynyl, and 5- to 12-membered heteroaryl comprising one to three hetero atoms selected from O, N, and S, wherein the 5- to 12-membered heteroaryl may be further substituted with one or two C₁-C₃alkyl groups or deuterated C₁-C₃alkyl groups;
R₅ is monocyclic or bicyclic including bridged bicyclic C₃-C₁₂heterocycloalkyl comprising one to three hetero atoms selected from O, N and S, wherein the C₃-C₁₂heterocycloalkyl may be further substituted with one or two groups chosen from halogen, hydroxy, alkoxy, cyano, C₁-C₄alkyl, C₁-C₄haloalkyl, and C₁-C₄dialkylamino;
R₆ is absent or is C₃-C₁₀ heterocycloalkyl including bridged heterobicycles comprising one or two hetero atoms selected from O, N and S, wherein the C₃-C₁₀ heterocycloalkyl may be further substituted with one or two groups chosen from C₁-C₄alkyl, deuterated C₁-C₄alkyl, C₁-C₄alkoxy, and C₁-C₄dialkylamino, and wherein the C₁-C₄alkyl may be further substituted with one to three groups chosen from halogen, —CN, hydroxyl, amino group, amide group, sulfoxide, sulfonamide, C₁-C₃alkoxy, and C₁-C₃acyl;
with the proviso that the compound of Formula (I) is not
N-(6-((5-bromo-2-((2-methoxy-5-methyl-4-(4-(4-methylpiperazin-1-yl)piperidin-1-yl)phenyl)amino)pyrimidin-4-yl)amino)quinoxalin-5-yl)methanesulfonamide;
N-(6-((5-bromo-2-((5-ethyl-2-methoxy-4-(4-(4-methylpiperazin-1-yl)piperidin-1-yl)phenyl)amino)pyrimidin-4-yl)amino)quinoxalin-5-yl)methanesulfonamide;
N-(6-((5-bromo-2-((2-methoxy-4-(4-(4-methylpiperazin-1-yl)piperidin-1-yl)phenyl)amino)pyrimidin-4-yl)amino)quinoxalin-5-yl)methanesulfonamide;
N-(6-((5-bromo-2-((2-methoxy-5-methyl-4-(4-(4-methylpiperazin-1-yl)piperidin-1-yl)phenyl)amino)pyrimidin-4-yl)amino)quinoxalin-5-yl)-N-methylmethanesulfonamide;
N-(6-((5-bromo-2-((5-ethyl-2-methoxy-6-(4-(4-methylpiperazin-1-yl)piperidin-1-yl)pyridin-3-yl)amino)pyrimidin-4-yl)amino)quinoxalin-5-yl)methanesulfonamide; or
N-(6-((5-bromo-2-((2-methoxy-5-(1-methyl-1H-pyrazol-4-yl)-4-morpholinophenyl)amino)pyrimidin-4-yl)amino)quinoxalin-5-yl)methanesulfonamide.

2. The compound according to claim 1, wherein R^{a} is chosen from H, CF₃, halogen, C₃-C₅cycloalkyl, and —C(O)OC₁-C₄alkyl.

3. The compound according to claim 2, wherein R^{a} is chosen from CF₃, Br, Cl, cyclopropyl, and —C(O)O-i-Pr.

4. (canceled)

5. (canceled)

6. (canceled)

7. (canceled)

8. The compound according to claim 1, wherein R, is H or methyl.

9. (canceled)

10. The compound according to claim 1, wherein R₂ is chosen from —NH₂, methyl, ethyl, and cyclopropyl.

11. (canceled)

12. (canceled)

13. (canceled)

14. The compound according to claim 1, wherein R₃ is chosen from methyl, CHF₂, —CD₃, and —CH₂CF₃.

15. (canceled)

16. (canceled)

17. The compound according to claim 1, wherein R₄ is chosen from H, methyl, ethyl, cyclopropyl, vinyl, and

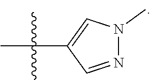 [SMILES: *c1cnn(C)c1]

18. (canceled)

19. (canceled)

20. (canceled)

21. (canceled)

22. (canceled)

23. The compound according to claim 1, wherein R₅ is chosen from

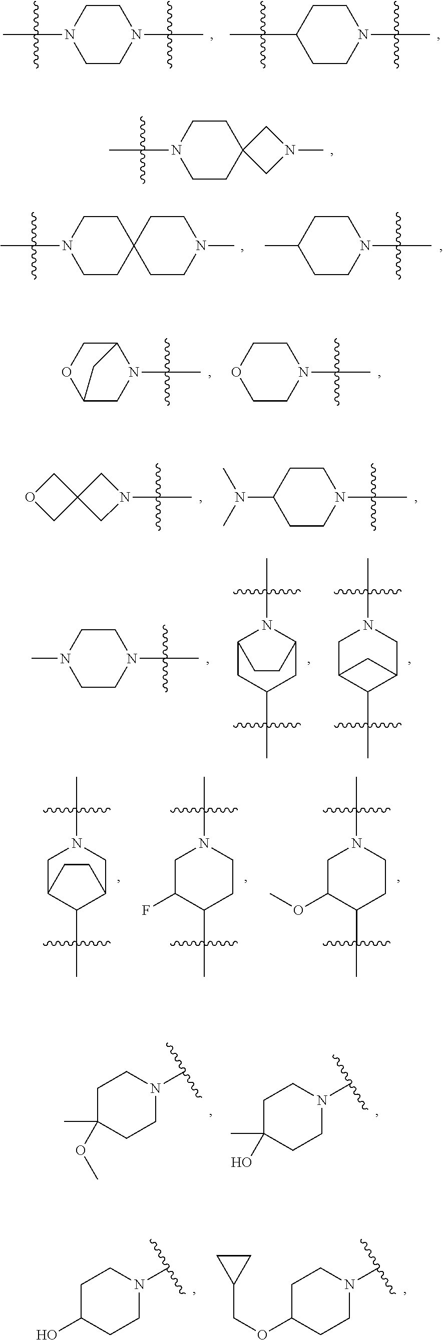

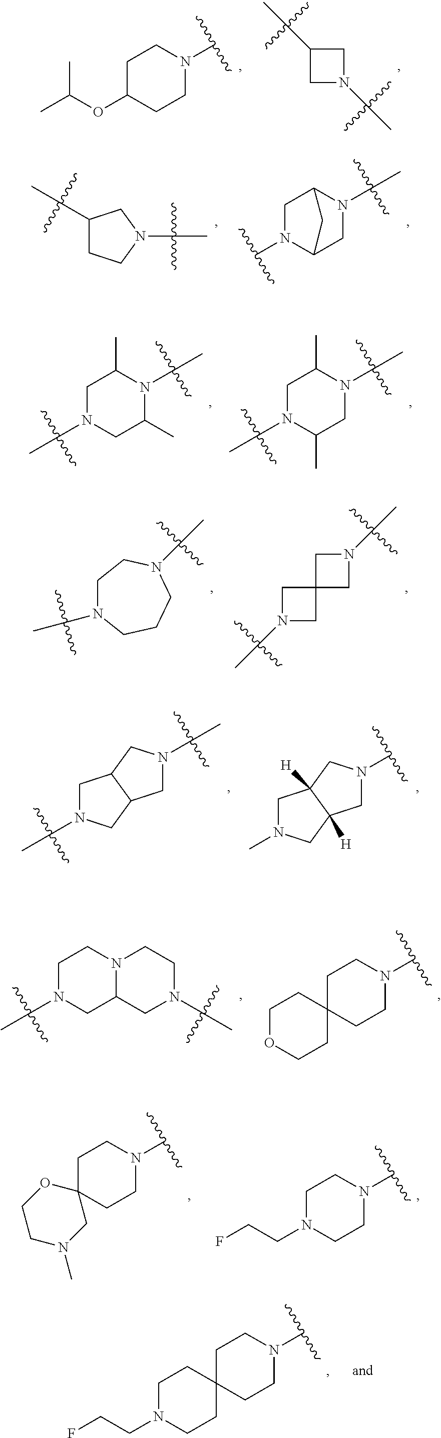

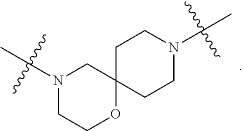 [SMILES: *N1CCC2(CC1)CN(*)CCO2]

24. (canceled)

25. The compound according to claim 1, wherein R₆ is absent or is chosen from

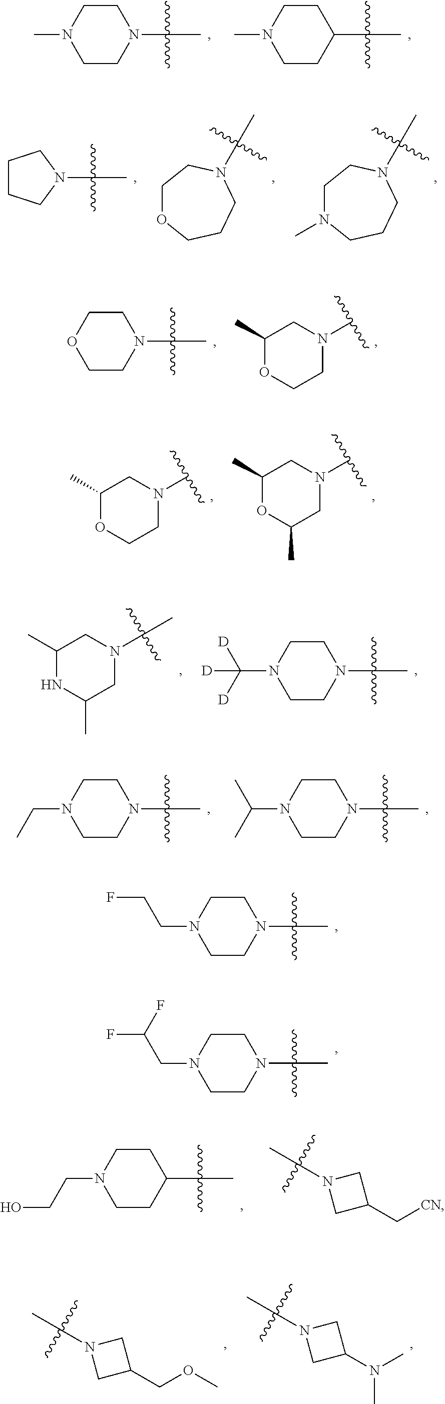

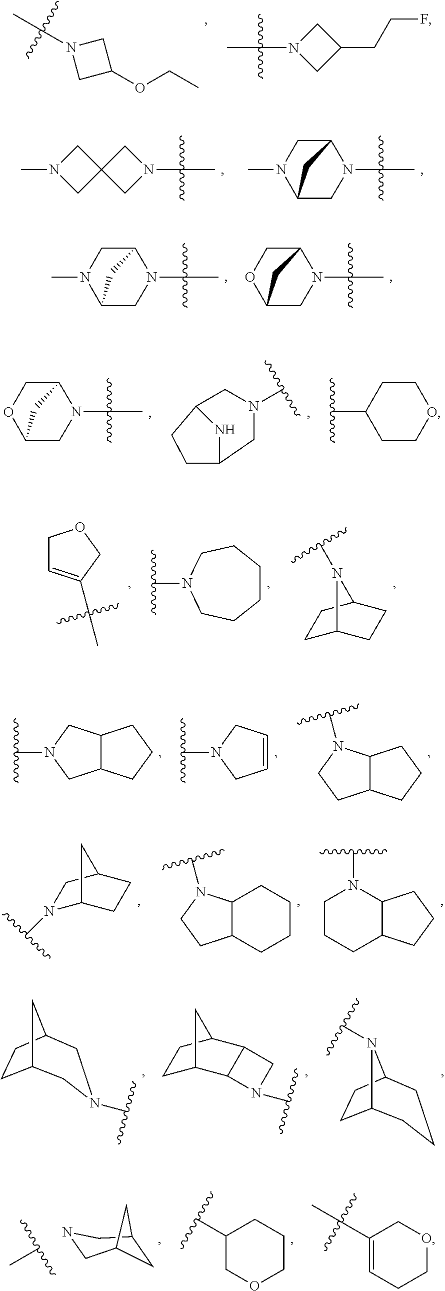

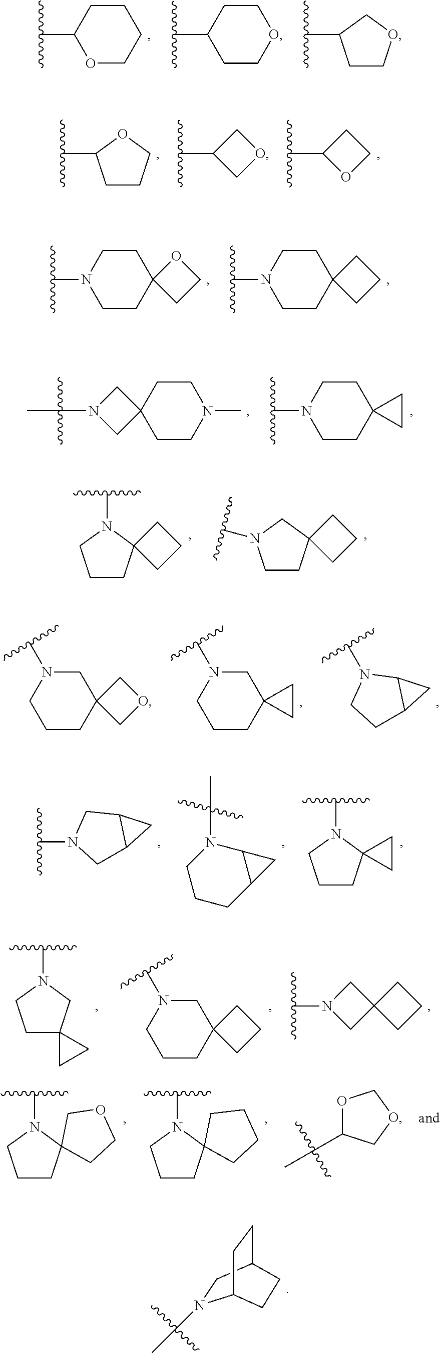

26. The compound according to claim 1, wherein the compound is chosen from:
N-(2-((4-((2-methoxy-5-methyl-4-(4-(4-methylpiperazin-1-yl)piperidin-1-yl)phenyl)amino)-1,3,5-triazin-2-yl)amino)phenyl)methanesulfonamide;
N-(6-((5-chloro-2-((2-methoxy-5-methyl-4-(4-(4-methylpiperazin-1-yl)piperidin-1-yl)phenyl)amino)pyrimidin-4-yl)amino)quinoxalin-5-yl)methanesulfonamide;
isopropyl 2-((2-methoxy-5-methyl-4-(4-(4-methylpiperazin-1-yl)piperidin-1-yl)phenyl)amino)-4-((5-(methylsulfonamido)quinoxalin-6-yl)amino)pyrimidine-5-carboxylate;
N-(6-((5-bromo-2-((2-methoxy-5-methyl-4-(4-(1-methylpiperidin-4-yl)piperazin-1-yl)phenyl)amino)pyrimidin-4-yl)amino)quinoxalin-5-yl)methanesulfonamide;
N-(6-((5-bromo-2-((4-(4-(dimethylamino)piperidin-1-yl)-2-methoxy-5-methylphenyl)amino)pyrimidin-4-yl)amino)quinoxalin-5-yl)methanesulfonamide;
N-(6-((5-bromo-2-((2-methoxy-5-methyl-4-(2-methyl-2,7-diazaspiro[3.5]nonan-7-yl)phenyl)amino)pyrimidin-4-yl)amino)quinoxalin-5-yl)methanesulfonamide;
N-(6-((5-bromo-2-((2-methoxy-5-methyl-4-(9-methyl-3,9-diazaspiro[5.5]undecan-3-yl)phenyl)amino)pyrimidin-4-yl)amino)quinoxalin-5-yl)methanesulfonamide;
N-(6-((5-bromo-2-((2-methoxy-5-methyl-4-(4-(pyrrolidin-1-yl)piperidin-1-yl)phenyl)amino)pyrimidin-4-yl)amino)quinoxalin-5-yl)methanesulfonamide;
N-(6-((4-((2-methoxy-5-methyl-4-(4-(4-methylpiperazin-1-yl)piperidin-1-yl)phenyl)amino)-1,3,5-triazin-2-yl)amino)quinoxalin-5-yl)methanesulfonamide;
N-(6-((4-((5-ethyl-2-methoxy-4-(4-(4-methylpiperazin-1-yl)piperidin-1-yl)phenyl)amino)-1,3,5-triazin-2-yl)amino)quinoxalin-5-yl)methanesulfonamide;
N-(3-((5-bromo-2-((5-ethyl-2-methoxy-4-(4-(4-methylpiperazin-1-yl)piperidin-1-yl)phenyl)amino)pyrimidin-4-yl)amino)quinolin-4-yl)methanesulfonamide;
N-(6-((2-((4-(4-(1,4-oxazepan-4-yl)piperidin-1-yl)-2-methoxy-5-vinylphenyl)amino)-5-bromopyrimidin-4-yl)amino)quinoxalin-5-yl)methanesulfonamide;
N-(6-((5-bromo-2-((5-ethyl-2-methoxy-4-(4-(4-methyl-1,4-diazepan-1-yl)piperidin-1-yl)phenyl)amino)pyrimidin-4-yl)amino)quinoxalin-5-yl)methanesulfonamide;
N-(3-((5-bromo-2-((2-methoxy-4-(4-(4-methylpiperazin-1-yl)piperidin-1-yl)-5-vinylphenyl)amino)pyrimidin-4-yl)amino)quinolin-4-yl)methanesulfonamide;
N-(6-((2-((4-(4-(1,4-oxazepan-4-yl)piperidin-1-yl)-5-ethyl-2-methoxyphenyl)amino)-5-bromopyrimidin-4-yl)amino)quinoxalin-5-yl)methanesulfonamide;
N-(6-((5-bromo-2-((2-methoxy-4-(4-(4-methyl-1,4-diazepan-1-yl)piperidin-1-yl)-5-vinylphenyl)amino)pyrimidin-4-yl)amino)quinoxalin-5-yl)methanesulfonamide;
N-(6-((5-bromo-2-((5-ethyl-6-(4-(4-methylpiperazin-1-yl)piperidin-1-yl)-2-(2,2,2-trifluoroethoxy)pyridin-3-yl)amino)pyrimidin-4-yl)amino)quinoxalin-5-yl)methanesulfonamide;
N-(6-((5-bromo-2-((2-methoxy-5-methyl-6-(4-(4-methylpiperazin-1-yl)piperidin-1-yl)pyridin-3-yl)amino)pyrimidin-4-yl)amino)quinoxalin-5-yl)methanesulfonamide;
N-(6-((5-bromo-2-((5-methyl-6-(4-(4-methylpiperazin-1-yl)piperidin-1-yl)-2-(2,2,2-trifluoroethoxy)pyridin-3-yl)amino)pyrimidin-4-yl)amino)quinoxalin-5-yl)methanesulfonamide;
N-(6-((5-bromo-2-((5-cyclopropyl-6-(4-(4-methylpiperazin-1-yl)piperidin-1-yl)-2-(2,2,2-trifluoroethoxy)pyridin-3-yl)amino)pyrimidin-4-yl)amino)quinoxalin-5-yl)methanesulfonamide;
N-(6-((2-((5-ethyl-2-methoxy-4-(4-(4-methylpiperazin-1-yl)piperidin-1-yl)phenyl)amino)-5-(trifluoromethyl)pyrimidin-4-yl)amino)quinoxalin-5-yl)methanesulfonamide;
N-(6-((5-cyclopropyl-2-((5-ethyl-2-methoxy-4-(4-(4-methylpiperazin-1-yl)piperidin-1-yl)phenyl)amino)pyrimidin-4-yl)amino)quinoxalin-5-yl)methanesulfonamide;
N-(3-((5-bromo-2-((5-ethyl-2-methoxy-4-(4-(4-methylpiperazin-1-yl)piperidin-1-yl)phenyl)amino)pyrimidin-4-yl)amino)-1,5-naphthyridin-4-yl)methanesulfonamide;
N-(6-((5-bromo-2-((5-ethyl-2-methoxy-4-(4-(4-methylpiperazin-1-yl)piperidin-1-yl)phenyl)amino)pyrimidin-4-yl)amino)quinolin-5-yl)methanesulfonamide;
N-(6-((5-bromo-2-((5-ethyl-2-methoxy-4-(4-morpholinopiperidin-1-yl)phenyl)amino)pyrimidin-4-yl)amino)quinoxalin-5-yl)methanesulfonamide;
N-(6-((5-bromo-2-((4-(4-(3,5-dimethylpiperazin-1-yl)piperidin-1-yl)-5-ethyl-2-methoxyphenyl)amino)pyrimidin-4-yl)amino)quinoxalin-5-yl)methanesulfonamide;
N-(6-((5-bromo-2-((5-ethyl-2-methoxy-4-(4-(4-methylpiperazin-1-yl)piperidin-1-yl)phenyl)amino)pyrimidin-4-yl)amino)quinoxalin-5-yl)sulfamide;
N-(6-((5-bromo-2-((5-ethyl-2-methoxy-4-(4-methylpiperidin-1-yl)phenyl)amino)pyrimidin-4-yl)amino)quinoxalin-5-yl)methanesulfonamide;
N-(6-((4-((5-ethyl-2-methoxy-4-(4-(4-methylpiperazin-1-yl)piperidin-1-yl)phenyl)amino)-1,3,5-triazin-2-yl)amino)quinoxalin-5-yl)methanesulfonamide;
N-(6-((4-((2-methoxy-5-methyl-4-(4-(4-methylpiperazin-1-yl)piperidin-1-yl)phenyl)amino)-1,3,5-triazin-2-yl)amino)quinoxalin-5-yl)methanesulfonamide;
N-(3-((4-((5-ethyl-2-methoxy-4-(4-(4-methylpiperazin-1-yl)piperidin-1-yl)phenyl)amino)-1,3,5-triazin-2-yl)amino)quinolin-4-yl)methanesulfonamide;
N-(2-((4-((5-ethyl-2-methoxy-4-(4-(4-methylpiperazin-1-yl)piperidin-1-yl)phenyl)amino)-1,3,5-triazin-2-yl)amino)naphthalen-1-yl)methanesulfonamide;
N-(2-((4-((5-ethyl-2-methoxy-4-(4-(4-methylpiperazin-1-yl)piperidin-1-yl)phenyl)amino)-1,3,5-triazin-2-yl)amino)phenyl)methanesulfonamide;
N-(6-((5-bromo-2-((5-ethyl-2-methoxy-4-(4-(4-methylpiperazin-1-yl)piperidin-1-yl)phenyl)amino)pyrimidin-4-yl)amino)quinoxalin-5-yl)ethanesulfonamide;
N-(6-((5-bromo-2-((5-ethyl-2-methoxy-4-(4-(4-methylpiperazin-1-yl)piperidin-1-yl)phenyl)amino)pyrimidin-4-yl)amino)quinoxalin-5-yl)cyclopropanesulfonamide;
N-(6-((5-bromo-2-((5-ethyl-2-(methoxy-d3)-4-(4-(4-methylpiperazin-1-yl)piperidin-1-yl)phenyl)amino)pyrimidin-4-yl)amino)quinoxalin-5-yl)methanesulfonamide;
N-(6-((5-bromo-2-((5-ethyl-2-methoxy-4-(4-(4-(methyl-d3)piperazin-1-yl)piperidin-1-yl)phenyl)amino)pyrimidin-4-yl)amino)quinoxalin-5-yl)methanesulfonamide;
N-(6-((5-bromo-2-((5-ethyl-4-(4-(4-ethylpiperazin-1-yl)piperidin-1-yl)-2-methoxyphenyl)amino)pyrimidin-4-yl)amino)quinoxalin-5-yl)methanesulfonamide;
N-(6-((5-bromo-2-((5-ethyl-4-(4-(4-isopropylpiperazin-1-yl)piperidin-1-yl)-2-methoxyphenyl)amino)pyrimidin-4-yl)amino)quinoxalin-5-yl)methanesulfonamide;
N-(6-((5-bromo-2-((5-ethyl-4-(1′-(2-hydroxyethyl)-[1,4′-bipiperidin]-4-yl)-2-methoxyphenyl)amino)pyrimidin-4-yl)amino)quinoxalin-5-yl)methanesulfonamide;
N-(6-((5-bromo-2-((2-methoxy-5-(1-methyl-1H-pyrazol-4-yl)-4-(2-oxa-6-azaspiro[3.3]heptan-6-yl)phenyl)amino)pyrimidin-4-yl)amino)quinoxalin-5-yl)methanesulfonamide;
N-(6-((5-bromo-2-((2-methoxy-5-(1-methyl-1H-pyrazol-4-yl)-4-(4-(4-methylpiperazin-1-yl)piperidin-1-yl)phenyl)amino)pyrimidin-4-yl)amino)quinoxalin-5-yl)methanesulfonamide;
N-(6-((5-bromo-2-((4-(4-(3-(cyanomethyl)azetidin-1-yl)piperidin-1-yl)-5-ethyl-2-methoxyphenyl)amino)pyrimidin-4-yl)amino)quinoxalin-5-yl)methanesulfonamide;
N-(6-((5-bromo-2-((5-ethyl-2-methoxy-4-(4-(3-(methoxymethyl)azetidin-1-yl)piperidin-1-yl)phenyl)amino)pyrimidin-4-yl)amino)quinoxalin-5-yl)methanesulfonamide;
N-(6-((5-bromo-2-((4-(4-(3-(dimethylamino)azetidin-1-yl)piperidin-1-yl)-5-ethyl-2-methoxyphenyl)amino)pyrimidin-4-yl)amino)quinoxalin-5-yl)methanesulfonamide;
N-(6-((5-bromo-2-((4-(4-(3-ethoxyazetidin-1-yl)piperidin-1-yl)-5-ethyl-2-methoxyphenyl)amino)pyrimidin-4-yl)amino)quinoxalin-5-yl)methanesulfonamide;
N-(6-((5-bromo-2-((2-methoxy-5-methyl-4-((1R,3s,5S)-3-(4-methylpiperazin-1-yl)-8-azabicyclo[3.2.1]octan-8-yl)phenyl)amino)pyrimidin-4-yl)amino)quinoxalin-5-yl)methanesulfonamide;
N-(6-((5-bromo-2-((2-methoxy-5-methyl-4-((1R,3r,5S)-3-(4-methylpiperazin-1-yl)-8-azabicyclo[3.2.1]octan-8-yl)phenyl)amino)pyrimidin-4-yl)amino)quinoxalin-5-yl)methanesulfonamide;
N-(6-((5-bromo-2-((2-methoxy-5-methyl-4-((1R,5S,6s)-6-(4-methylpiperazin-1-yl)-3-azabicyclo[3.1.1]heptan-3-yl)phenyl)amino)pyrimidin-4-yl)amino)quinoxalin-5-yl)methanesulfonamide;
N-(6-((5-bromo-2-((2-methoxy-5-methyl-4-((1R,5S,6r)-6-(4-methylpiperazin-1-yl)-3-azabicyclo[3.1.1]heptan-3-yl)phenyl)amino)pyrimidin-4-yl)amino)quinoxalin-5-yl)methanesulfonamide;
N-(6-((5-bromo-2-((2-methoxy-5-methyl-4-((1R,5S,8s)-8-(4-methylpiperazin-1-yl)-3-azabicyclo[3.2.1]octan-3-yl)phenyl)amino)pyrimidin-4-yl)amino)quinoxalin-5-yl)methanesulfonamide;
N-(6-((5-bromo-2-((2-methoxy-5-methyl-4-((1R,5S,8r)-8-(4-methylpiperazin-1-yl)-3-azabicyclo[3.2.1]octan-3-yl)phenyl)amino)pyrimidin-4-yl)amino)quinoxalin-5-yl)methanesulfonamide;
N-(6-((5-bromo-2-((4-((3R,4R)-3-fluoro-4-(4-methylpiperazin-1-yl)piperidin-1-yl)-2-methoxy-5-methylphenyl)amino)pyrimidin-4-yl)amino)quinoxalin-5-yl)methanesulfonamide;
N-(6-((5-bromo-2-((4-((3S,4S)-3-fluoro-4-(4-methylpiperazin-1-yl)piperidin-1-yl)-2-methoxy-5-methylphenyl)amino)pyrimidin-4-yl)amino)quinoxalin-5-yl)methanesulfonamide;
N-(6-((5-chloro-2-((4-(4-(4-(2-fluoroethyl)piperazin-1-yl)piperidin-1-yl)-2-methoxy-5-methylphenyl)amino)pyrimidin-4-yl)amino)quinoxalin-5-yl)methanesulfonamide;
N-(6-((5-chloro-2-((4-(4-(4-(2,2-difluoroethyl)piperazin-1-yl)piperidin-1-yl)-2-methoxy-5-methylphenyl)amino)pyrimidin-4-yl)amino)quinoxalin-5-yl)methanesulfonamide;
N-(6-((2-((2-methoxy-5-methyl-4-(4-((1R,4R)-5-methyl-2,5-diazabicyclo[2.2.1]heptan-2-yl)piperidin-1-yl)phenyl)amino)-5-methylpyrimidin-4-yl)amino)quinoxalin-5-yl)methanesulfonamide;
N-(6-((2-((2-methoxy-5-methyl-4-(4-((1 S,4S)-5-methyl-2,5-diazabicyclo[2.2.1]heptan-2-yl)piperidin-1-yl)phenyl)amino)-5-methylpyrimidin-4-yl)amino)quinoxalin-5-yl)methanesulfonamide;
N-(6-((5-chloro-2-((5-ethyl-2-methoxy-4-(4-((1R,4R)-5-methyl-2,5-diazabicyclo[2.2.1]heptan-2-yl)piperidin-1-yl)phenyl)amino)pyrimidin-4-yl)amino)quinoxalin-5-yl)methanesulfonamide;
N-(6-((5-chloro-2-((5-ethyl-2-methoxy-4-(4-((1S,4S)-5-methyl-2,5-diazabicyclo[2.2.1]heptan-2-yl)piperidin-1-yl)phenyl)amino)pyrimidin-4-yl)amino)quinoxalin-5-yl)methanesulfonamide;
N-(6-((5-bromo-2-((5-ethyl-2-methoxy-4-((3aR,6aS)-5-methylhexahydropyrrolo[3,4-c]pyrrol-2(1H)-yl)phenyl)amino)pyrimidin-4-yl)amino)quinoxalin-5-yl)methanesulfonamide;
N-(6-((5-bromo-2-((2-methoxy-5-methyl-4-(4-methylpiperazin-1-yl)phenyl)amino)pyrimidin-4-yl)amino)quinoxalin-5-yl)methanesulfonamide;
N-(6-((2-((5-ethyl-2-methoxy-4-(4-(4-methylpiperazin-1-yl)piperidin-1-yl)phenyl)amino)-5-methylpyrimidin-4-yl)amino)quinoxalin-5-yl)methanesulfonamide;
N-(6-((5-chloro-2-((2-methoxy-5-(1-methyl-1H-pyrazol-4-yl)-4-(4-(4-methylpiperazin-1-yl)piperidin-1-yl)phenyl)amino)pyrimidin-4-yl)amino)quinoxalin-5-yl)methanesulfonamide;
N-(6-((5-bromo-2-((2-methoxy-5-(1-methyl-1H-pyrazol-4-yl)-4-(4-(4-methylpiperazin-1-yl)piperidin-1-yl)phenyl)amino)pyrimidin-4-yl)amino)quinoxalin-5-yl)ethanesulfonamide;
N-(6-((5-bromo-2-((2-methoxy-5-(1-methyl-1H-pyrazol-4-yl)-4-(4-(4-methylpiperazin-1-yl)piperidin-1-yl)phenyl)amino)pyrimidin-4-yl)amino)quinoxalin-5-yl)-N-methylmethanesulfonamide;
N-(6-((5-bromo-2-((2-methoxy-5-(1-methyl-1H-pyrazol-4-yl)-4-(4-(4-(methyl-d3)piperazin-1-yl)piperidin-1-yl)phenyl)amino)pyrimidin-4-yl)amino)quinoxalin-5-yl)methanesulfonamide;
N-(6-((5-bromo-2-((2-(methoxy-d3)-5-(1-methyl-1H-pyrazol-4-yl)-4-(4-(4-methylpiperazin-1-yl)piperidin-1-yl)phenyl)amino)pyrimidin-4-yl)amino)quinoxalin-5-yl)methanesulfonamide;
N-(6-((5-bromo-2-((2-methoxy-5-(1-(methyl-d3)-1H-pyrazol-4-yl)-4-(4-(4-methylpiperazin-1-yl)piperidin-1-yl)phenyl)amino)pyrimidin-4-yl)amino)quinoxalin-5-yl)methanesulfonamide;
N-(6-((2-((2-methoxy-5-(1-methyl-1H-pyrazol-4-yl)-4-(4-(4-methylpiperazin-1-yl)piperidin-1-yl)phenyl)amino)-5-(trifluoromethyl)pyrimidin-4-yl)amino)quinoxalin-5-yl)methanesulfonamide;
N-(6-((5-isopropyl-2-((2-methoxy-5-(1-methyl-1H-pyrazol-4-yl)-4-(4-(4-methylpiperazin-1-yl)piperidin-1-yl)phenyl)amino)pyrimidin-4-yl)amino)quinoxalin-5-yl)methanesulfonamide;
N-(6-((5-cyclopropyl-2-((2-methoxy-5-(1-methyl-1H-pyrazol-4-yl)-4-(4-(4-methylpiperazin-1-yl)piperidin-1-yl)phenyl)amino)pyrimidin-4-yl)amino)quinoxalin-5-yl)methanesulfonamide;
N-(6-((5-bromo-2-((2-methoxy-5-(1-methyl-1H-pyrazol-4-yl)-6-(4-(4-methylpiperazin-1-yl)piperidin-1-yl)pyridin-3-yl)amino)pyrimidin-4-yl)amino)quinoxalin-5-yl)methanesulfonamide;
N-(6-((5-bromo-2-((2-methoxy-4-(4-methoxy-4-methylpiperidin-1-yl)-5-(1-methyl-1H-pyrazol-4-yl)phenyl)amino)pyrimidin-4-yl)amino)quinoxalin-5-yl)methanesulfonamide;
N-(2-((5-bromo-2-((2-methoxy-5-(1-methyl-1H-pyrazol-4-yl)-4-(3-oxa-9-azaspiro[5.5]undecan-9-yl)phenyl)amino)pyrimidin-4-yl)amino)phenyl)methanesulfonamide;
(S)—N-(6-((5-bromo-2-((2-methoxy-5-(1-methyl-1H-pyrazol-4-yl)-4-(4-(2-methylmorpholino)piperidin-1-yl)phenyl)amino)pyrimidin-4-yl)amino)quinoxalin-5-yl)methanesulfonamide;
(R)—N-(6-((5-bromo-2-((2-methoxy-5-(1-methyl-1H-pyrazol-4-yl)-4-(4-(2-methylmorpholino)piperidin-1-yl)phenyl)amino)pyrimidin-4-yl)amino)quinoxalin-5-yl)methanesulfonamide;
N-(6-((5-bromo-2-((4-(4-(cyclopropylmethoxy)piperidin-1-yl)-2-methoxy-5-(1-methyl-1H-pyrazol-4-yl)phenyl)amino)pyrimidin-4-yl)amino)quinoxalin-5-yl)methanesulfonamide;
N-(6-((5-bromo-2-((4-(4-isopropoxypiperidin-1-yl)-2-methoxy-5-(1-methyl-1H-pyrazol-4-yl)phenyl)amino)pyrimidin-4-yl)amino)quinoxalin-5-yl)methanesulfonamide;
N-(6-((5-bromo-2-((2-methoxy-5-(1-methyl-1H-pyrazol-4-yl)-4-(4-methyl-1-oxa-4,9-diazaspiro[5.5]undecan-9-yl)phenyl)amino)pyrimidin-4-yl)amino)quinoxalin-5-yl)methanesulfonamide;
N-(2-((5-bromo-2-((4-(9-(2-fluoroethyl)-3,9-diazaspiro[5.5]undecan-3-yl)-2-methoxy-5-(1-methyl-1H-pyrazol-4-yl)phenyl)amino)pyrimidin-4-yl)amino)phenyl) methanesulfonamide;
N-(6-((5-bromo-2-((4-(4-hydroxy-4-methylpiperidin-1-yl)-2-methoxy-5-(1-methyl-1H-pyrazol-4-yl)phenyl)amino)pyrimidin-4-yl)amino)quinoxalin-5-yl)methanesulfonamide;
N-(2-((5-bromo-2-((2-methoxy-5-(1-methyl-1H-pyrazol-4-yl)-4-(4-morpholinopiperidin-1-yl)phenyl)amino)pyrimidin-4-yl)amino)phenyl)methanesulfonamide;
N-(6-((5-bromo-2-((4-(4-hydroxypiperidin-1-yl)-2-methoxy-5-(1-methyl-1H-pyrazol-4-yl)phenyl)amino)pyrimidin-4-yl)amino)quinoxalin-5-yl)methanesulfonamide;
N-(6-((5-bromo-2-((5-(1-methyl-1H-pyrazol-4-yl)-4-(4-(4-methylpiperazin-1-yl)piperidin-1-yl)-2-(2,2,2-trifluoroethoxy)phenyl)amino)pyrimidin-4-yl)amino)quinoxalin-5-yl)methanesulfonamide;
N-(2-((5-bromo-2-((2-methoxy-5-(1-methyl-1H-pyrazol-4-yl)-4-(4-(4-methylpiperazin-1-yl)piperidin-1-yl)phenyl)amino)pyrimidin-4-yl)amino)phenyl)methanesulfonamide;
N-(6-((5-bromo-2-((4-(4-(2-fluoroethyl)piperazin-1-yl)-2-methoxy-5-(1-methyl-1H-pyrazol-4-yl)phenyl)amino)pyrimidin-4-yl)amino)quinoxalin-5-yl)methanesulfonamide;
N-(6-((5-bromo-2-((2-methoxy-5-(1-methyl-1H-pyrazol-4-yl)-4-(4-(4-methylpiperazin-1-yl)piperidin-1-yl)phenyl)amino)pyrimidin-4-yl)amino)quinoxalin-5-yl)cyclopropanesulfonamide;
N-(6-((5-bromo-2-((2-methoxy-5-(1-methyl-1H-pyrazol-4-yl)-4-(4-(pyrrolidin-1-yl)piperidin-1-yl)phenyl)amino)pyrimidin-4-yl)amino)quinoxalin-5-yl)methanesulfonamide;
N-(6-((5-bromo-2-((4-(4-(dimethylamino)piperidin-1-yl)-2-methoxy-5-(1-methyl-1H-pyrazol-4-yl)phenyl)amino)pyrimidin-4-yl)amino)quinoxalin-5-yl)methanesulfonamide;
N-(6-((5-bromo-2-((2-methoxy-5-(1-methyl-1H-pyrazol-4-yl)-4-(1-methylpiperidin-4-yl)phenyl)amino)pyrimidin-4-yl)amino)quinoxalin-5-yl)methanesulfonamide;
N-(6-((2-((2-methoxy-5-(1-methyl-1H-pyrazol-4-yl)-4-(4-(4-methylpiperazin-1-yl)piperidin-1-yl)phenyl)amino)-5-methylpyrimidin-4-yl)amino)quinoxalin-5-yl)methanesulfonamide;
N-(6-((5-bromo-2-((2-methoxy-5-(1-methyl-1H-pyrazol-4-yl)-4-(4-(1-methylpiperidin-4-yl)piperazin-1-yl)phenyl)amino)pyrimidin-4-yl)amino)quinoxalin-5-yl)methanesulfonamide; and
N-(6-((2-((4-(4-(3,8-diazabicyclo[3.2.1]octan-3-yl)piperidin-1-yl)-2-methoxy-5-(1-methyl-1H-pyrazol-4-yl)phenyl)amino)-5-bromopyrimidin-4-yl)amino)quinoxalin-5-yl)methanesulfonamide.

27. A pharmaceutical composition comprising a therapeutically effective amount of the at least one compound according to claim 1 and at least one additional component selected from pharmaceutically acceptable carriers, pharmaceutically acceptable vehicles, and pharmaceutically acceptable excipients.

28. A method of treating cancer in a subject comprising administering to the subject a therapeutically effective amount of the at least one compound of claim 1 or the pharmaceutical composition of claim 27.

29. The method of claim 28, wherein the cancer is associated with one or more EGFR mutations.

30. The method of claim 28, wherein the compound is a mutant-selective EGFR inhibitor.

31. The method of claim 28, wherein cancer is chosen from breast cancer, lung cancer, pancreatic cancer, colon cancer, head and neck cancer, renal cell carcinoma, squamous, cell carcinoma, thyroid cancer, gall bladder cancer, thyroid cancer, bile duct cancer, ovarian cancer, endometrial cancer, prostate cancer, and esophageal cancer.

32. (canceled)

33. (canceled)

34. (canceled)

35. (canceled)

36. (canceled)

37. (canceled)

38. (canceled)

39. (canceled)

40. The method of claim 28, comprising administering to the subject the at least one compound of claim 1 in combination with at least one is administered additional agent.

41. The method of claim 40, wherein the at least one compound and the at least one additional agent are administered concomitantly.

42. The method of claim 40, wherein the at least one compound and the at least one additional agent are administered sequentially.

43. The method of claim 40, wherein the at least one additional agent is chosen from osimertinib, cetuximab, gefitinib, lapatinib, and erlotinib.

44. The method of claim 28, wherein the at least one compound inhibits one or more mutations selected from the group consisting of an EGFR mutation, FLT3, and FLT3 mutation.

45. The method of claim 44, wherein the EGFR mutation is one or more selected from the group consisting of EGFR dell 9, EGFR del19/T790M, EGFR del19/T790M/C797S, EGFR L858R, EGFR L858R/T790MS, and EGFR L858R/T790M/C797S.

46. (canceled)

47. The method of claim 28, comprising administering to the subject the pharmaceutical composition of claim 27 in combination with at least one additional agent.

48. The method of claim 47, wherein the pharmaceutical composition and the at least one additional agent are administered concomitantly.

49. The method according to claim 47, wherein the pharmaceutical composition and the at least one additional agent are administered sequentially.
